(19) United States
(12) Patent Application Publication
(10) Pub. No.: US 2026/0345916 A1
(43) Pub. Date: Nov. 5, 2026

(54) DOSE DELIVERY MECHANISM

(71) Applicant: medmix Switzerland AG, Haag (Rheintal) (CH)

(72) Inventors: Joachim KEITEL, Esslingen (DE); Herbert BECHTOLD, Denkingen (DE); Kenneth Allen FOCHT, Needham, MA (US); Peter Calvin COSTELLO, Raynham, MA (US); Daniel P. SMITH, Portsmouth, RI (US)

(21) Appl. No.: 19/124,778

(22) PCT Filed: Oct. 27, 2023
(86) PCT No.: PCT/EP2023/080161

§ 371 Date: Apr. 25, 2025

Related U.S. Application Data
(63) Continuation of application No. 17/981,231, filed on Nov. 4, 2022.

(30) Foreign Application Priority Data
Apr. 27, 2023 (WO) PCT/EP2023/061173
Apr. 27, 2023 (WO) PCT/EP2023/061175
Apr. 27, 2023 (WO) PCT/EP2023/061183
Apr. 27, 2023 (WO) PCT/EP2023/061184
Apr. 27, 2023 (WO) PCT/EP2023/061187
Apr. 27, 2023 (WO) PCT/EP2023/061193

(51) Int. Cl. A61M 5/315; A61M 5/24

(52) U.S. Cl. CPC A61M 5/31585; A61M 5/24; A61M 5/31541; A61M 5/31551; A61M 2005/2407; A61M 2005/2437; A61M 2005/2444

(57) ABSTRACT

A dose delivery mechanism includes a housing, a piston rod and an adjusting element. The housing connects to a medicament container sealed by a plunger. The dose delivery mechanism thereby is in a preassembled state, in which the adjusting element adjusts an axial position of the piston rod with respect to the housing upon a rotation of the adjusting element. The dose delivery mechanism transfers from the preassembled state into an assembled state, in which the dose delivery mechanism moves the piston rod axially in a proximal direction with respect to the housing during dose delivery to expel a medicament from the medicament container. The adjusting element includes a radially outermost surface that is to be gripped by a user for setting a dose to be delivered in the assembled state.

### CROSS REFERENCE TO RELATED APPLICATIONS

This application is a U.S. national stage application of International Application No. PCT/EP2023/080161, filed on Oct. 27, 2023, which claims priority to U.S. application Ser. No. 17/981231, filed on Nov. 4, 2022, International Application No. PCT/EP2023/061173, filed on Apr. 27, 2023, International Application No. PCT/EP2023/061175, filed on Apr. 27, 2023, International Application No. PCT/EP2023/061183, filed on Apr. 27, 2023, International Application No. PCT/EP2023/061184, filed on Apr. 27, 2023, International Application No. PCT/EP2023/061187, filed on Apr. 27, 2023 and International Application No. PCT/EP2023/061193, filed on Apr. 27, 2023, the contents of each of which are hereby incorporated by reference.

### BACKGROUND

### Technical Field

The present disclosure relates to a dose delivery mechanism for a medicament de-livery device, a medicament delivery device having a dose delivery mechanism and a method for adjusting a dose delivery mechanism.

### Background Information

Medicament delivery devices, such as injection devices, that are used to deliver a liquid medicament to a patient usually comprise a medicament container holding the medicament and a dose delivery mechanism configured to expel a predefined dose of medicament from that container. At a distal side facing away from the delivery site, the medicament container usually comprises a movable plunger that seals the medicament container and that is moved in a proximal direction towards the injection site to expel the medicament from the container. To move the plunger, the dose delivery mechanism usually comprises a piston rod that acts on the plunger by moving the plunger in the proximal direction. The dose to be delivered then is defined by the axial movement of the plunger within the medicament container.

The position of the plunger within the medicament container is typically specified with a manufacturing tolerance of ±0.4 mm or ±0.5 mm. After assembly of the medicament container to the dose delivery mechanism, this tolerance might cause a bearing that is located at the piston rod and configured to push upon the plunger to contact and pressurize the plunger after final assembly or to be located at a distance from the plunger thus leaving a gap between bearing and plunger.

### SUMMARY

It has been determined that when contacting the plunger, the piston rod should not significantly compress the plunger since a permanent pressure onto the plunger between final assembly and first use of the device is not desirable. For example, a user would experience a loss of medicament when first attaching a needle to the medicament container. Furthermore, expansion of the medicament during transport, for example due to temperature and/or pressure changes, can damage the device.

When locating the bearing of the piston rod at a distance from the plunger, a gap between the plunger and the bearing varies from pen to pen due to manufacturing tolerances. The medicament delivery device then usually is primed by a user at least before delivering the first dose. The user attaches a needle to the medicament container, sets a dose, and expels the set dose into air. Depending on the size of the gap and the size of the dose used for priming, the user may have to repeat this procedure until at least some medicament is expelled. With a device primed by the user, a position of the plunger after attachment of the medicament container to the dose delivery mechanism should be large enough to be compatible with all possible manufacturing tolerances of the plunger position. At the same time, it should be small enough to avoid an excessive number of priming steps and/or to avoid an excessive amount of medicament being expelled during priming.

Furthermore, a user might forget to prime the device before use. This would lead to an incorrect amount of medicament being expelled since the distance that the piston rod moves to close the gap does not contribute to medicament delivery. The larger the gap, the larger is the inaccuracy of the first dose delivery.

Accordingly, there is a need to adjust the piston rod of the dose delivery mechanism at a well-defined position during assembly of the medicament delivery device.

It is thus an objective of the present disclosure to provide a dose delivery mechanism for a medicament delivery device, a medicament delivery device having a dose delivery mechanism and a method that allows for a reliable and simple adjustment of a position of the piston rod.

The present disclosure provides a dose delivery mechanism for a medicament de-livery device, a medicament delivery device having a dose delivery mechanism and a method for adjusting a dose delivery mechanism according to the independent claims. Embodiments are given in the dependent claims, the description and the drawings.

In one aspect, the present disclosure is directed at a dose delivery mechanism for a medicament delivery device comprising: a housing; a piston rod; and an adjusting element, wherein the housing is configured to connect to a medicament container sealed by a plunger. The dose delivery mechanism thereby is in a preassembled state, wherein, in the preassembled state, the adjusting element forms a means for adjusting an axial position of the piston rod with respect to the housing upon a rotation of the adjusting element with respect to the housing. Furthermore, the dose delivery mechanism is configured to transfer from the preassembled state into an assembled state, wherein, in the assembled state, the dose delivery mechanism is configured to move the piston rod axially in a proximal direction with respect to the housing during dose delivery such that the piston rod exerts an axial force in the proximal direction on the plunger of the medicament container to expel a medicament from the medicament container. The adjusting element comprises a radially outermost surface that is configured to be gripped by a user for setting a dose to be delivered in the assembled state, wherein the adjusting element completely comprises the radially outermost surface.

According to an embodiment, the adjusting element is configured to perform the rotation in the preassembled state after having been displaced from a rest position into an adjusting position that deviates from the rest position, wherein, for example, the adjusting element is configured to be pushed in the proximal direction from the rest position into the adjusting position.

According to an embodiment, the dose delivery mechanism is configured to transfer the rotation of the adjusting element into the axial movement of the piston rod via a single threaded connection in the preassembled state, wherein the threaded connection comprises a first threaded element that is threadedly engaged with a second threaded element. With some embodiments, a pitch of the single threaded connection can define a ratio of an axial distance to a circumferential distance and the piston rod can travel the axial distance with respect to the housing upon rotation of the adjusting element by the circumferential distance.

According to an embodiment, an outer rim of the adjusting element is not covered by other members of the dose delivery mechanism, such as the housing, in a radial direction perpendicular to a longitudinal axis of the dose delivery mechanism, such as in all radial directions.

According to an embodiment, in the preassembled state, the adjusting element is configured to perform a rotation with respect to the housing, wherein the rotation of the adjusting element causes an axial movement of the piston rod for adjusting an axial position of the piston rod with respect to the housing prior to transfer of the dose delivery mechanism from the preassembled state into the assembled state.

According to an embodiment, the dose delivery mechanism is assembled from the same components in the preassembled state and in the assembled state.

According to an embodiment, the transfer of the dose delivery mechanism from the preassembled state into the assembled state does not comprise an addition and/or removal of a component of the dose delivery mechanism.

According to an embodiment, all remaining components of the dose delivery mechanism except for the adjusting element, the piston rod and components axially fixed to the piston rod are axially positioned in a zero dose position with respect to the housing in the preassembled state, wherein the remaining components are configured to take up the zero dose position prior to setting a dose with the dose delivery mechanism in the assembled state.

According to an embodiment, the adjusting element is configured to be transferred from a preassembled position into an assembled position with respect to the housing when transferring the dose delivery mechanism from the preassembled state into the assembled state.

With some embodiments, the transfer of the adjusting element from the preassembled position into the assembled position causes a transfer of the dose delivery mechanism from the preassembled state into the assembled state.

According to an embodiment, the adjusting element is in a first axial position with respect to a retaining member of the dose delivery mechanism in the preassembled state, wherein the adjusting element is configured to move axially from the first axial position into a second axial position with respect to the retaining member upon transfer of the dose delivery mechanism from the preassembled state into the assembled state.

According to an embodiment, the retaining member is formed by a coupling element that rotationally fixes the adjusting element to the piston rod.

According to an embodiment, the adjusting element is rotationally fixed with respect to the retaining member in the preassembled state.

According to an embodiment, the retaining member is configured to be moved axially, for example in the proximal direction, together with the adjusting element into an adjusting position with respect to the housing, wherein the adjusting element is configured to perform the rotation with respect to the housing in the adjusting position to cause the axial movement of the piston rod.

According to an embodiment, axial movement of the retaining member opens a clutch mechanism to rotationally decouple a further member from the adjusting element during the rotation with respect to the housing in the adjusting position.

According to an embodiment, the further member is threadedly coupled to the piston rod and/or axially fixed with respect to the housing.

According to an embodiment, the further member is formed by a dosing member of the dose delivery mechanism.

According to an embodiment, the adjusting element is configured to be moved axially into the proximal direction when being in the adjusting position, wherein further proximal movement of the adjusting element from the adjusting position transfers the adjusting element from the first axial position into the second axial position with respect to the retaining member and transfers the dose delivery mechanism from the preassembled state into the assembled state.

According to an embodiment, the adjusting element is axially coupled to the retaining member via a latching mechanism.

According to an embodiment, the latching mechanism is configured to transfer axial movement of the adjusting element into axial movement of the retaining member during movement of the adjusting element, for example into the adjusting position.

According to an embodiment, the latching mechanism is configured to allow proximal movement of the adjusting element with respect to the retaining member in the adjusting position to transfer the dose delivery mechanism from the preassembled state into the assembled state.

According to an embodiment, the dose delivery mechanism comprises a clutch mechanism having a first clutch member and a second clutch member, wherein the clutch mechanism connects the adjusting element to a further member. The clutch mechanism is opened in the preassembled state during the rotation of the adjusting element thus allowing a rotation of the adjusting element with respect to the further member and the clutch mechanism is opened in the assembled state during dose delivery thus allowing a rotation of the adjusting element with respect to the further member.

According to an embodiment, the clutch mechanism is closed in the assembled state during dose setting.

According to an embodiment, the clutch mechanism is biased into a closed configuration in the preassembled state, wherein the adjusting element is configured to be axially moved with respect to the housing, for example in the proximal direction, in the preassembled state to open the clutch mechanism.

According to an embodiment, the dose delivery mechanism comprises a dose definition mechanism that defines doses settable by a user in the assembled state, wherein the dose definition mechanism defines rotational positions of the adjusting element with respect to the housing that correspond to the doses settable by the user.

According to an embodiment, the dose definition mechanism is actuated during the rotation of the adjusting element in the preassembled state.

According to an embodiment, a distal end surface of the dose delivery mechanism forms part of the adjusting element or is rigidly connected with the adjusting element.

According to an embodiment, the adjusting element is located at a distal end of the dose delivery mechanism.

According to an embodiment, the adjusting element forms at least part of a knob that is placed at a distal end of the dose delivery mechanism.

According to an embodiment, the adjusting element protrudes in a distal direction from the housing in the preassembled state.

According to an embodiment, the adjusting element protrudes in a distal direction from the remaining members of the dose delivery mechanism in the preassembled state.

The present disclosure is also directed at a medicament delivery device having a dose delivery mechanism according to the present disclosure and a medicament container attached to the dose delivery mechanism, the medicament container comprising a plunger, wherein a bearing located at the piston rod is positioned at a predetermined distance with respect to the plunger and the predetermined distance is larger than zero.

According to an embodiment, the medicament container comprises a medicament chamber that is sealed by the plunger, wherein the medicament chamber comprises a medicament and air.

Furthermore, the present disclosure is directed at a method for adjusting a position of a piston rod of a dose delivery mechanism according to the present disclosure, the method comprising at least the following steps:
providing the dose delivery mechanism in the preassembled state;
adjusting the axial position of the piston rod with respect to the housing by rotating the adjusting element in the preassembled state and thereby causing an axial movement of the piston rod with respect to the housing;
transferring the dose delivery mechanism from the preassembled state into the assembled state.

According to an embodiment, the method comprises the step of moving the adjusting element from a rest position with respect to the housing into an adjusting position, wherein the step of rotating the adjusting element is performed with the adjusting element being in the adjusting position.

According to an embodiment, the adjusting element is moved axially in a proximal direction from the preassembled position into the adjusting position.

According to an embodiment, the dose delivery mechanism comprises a stop acting between the adjusting element and the housing, wherein the stop exerts a resistance against the moving of the adjusting element when the adjusting element reaches the adjusting position.

According to an embodiment, the method comprises the step of overcoming the resistance exerted by the stop to further move the adjusting element to transfer the dose de-livery mechanism from the preassembled state into the assembled state.

According to an embodiment, the adjusting element is biased into the preassembled position by a biasing force and the method comprises the step of holding the adjusting element in the adjusting position against the biasing force during the rotating of the adjusting element.

According to an embodiment, the dose delivery mechanism comprises a clutch mechanism, wherein clutch mechanism is closed when the adjusting element is in the preassembled state and wherein the step of moving the adjusting element from the preassembled position into the adjusting position opens the clutch mechanism.

According to an embodiment, the dose delivery mechanism is provided with the adjusting element in a first axial position with respect to a retaining member of the dose de-livery mechanism and the step of moving of the adjusting element from the preassembled position into the adjusting position causes the retaining member to axially move together with the adjusting element.

According to an embodiment, the dose delivery mechanism is provided in the preassembled state with all components of the dose delivery mechanism connected to each other.

According to an embodiment, the dose delivery mechanism comprises a dose definition mechanism that defines doses settable by a user in an assembled state of the dose de-livery mechanism, wherein the dose definition mechanism defines rotational positions of the adjusting element with respect to the housing that correspond to the doses settable by the user The method further comprises the step of actuating the dose definition mechanism during the rotating of the adjusting element in the preassembled state.

According to an embodiment, the step of actuating the dose definition mechanism generates a feedback signal, such as an audible and/or tactile feedback signal.

According to an embodiment, the dose delivery mechanism is provided in the preassembled state with the medicament container attached.

According to an embodiment, the axial position of the piston rod is adjusted to place a bearing located at the piston rod in contact with a surface of the plunger of the medicament container.

According to an embodiment, the axial position of the piston rod is adjusted to move the plunger within the medicament container to expel air from the medicament container.

According to an embodiment, the piston rod is axially moved during the adjusting of the axial position until the rotating of the adjusting element requires a predetermined torque.

According to an embodiment, the method comprises the step of measuring a torque required for the rotating of the adjusting element. The rotating of the adjusting element can be stopped when the torque reaches a predetermined threshold.

According to an embodiment, the method comprises the step of transferring the adjusting element from a preassembled position into an assembled position.

According to an embodiment, the method comprises the step of blocking, such as irreversibly blocking, the adjusting element in the assembled position from returning into the preassembled position with respect to the housing.

According to an embodiment, the method comprises the step of transferring the adjusting element from a first axial position with respect to a retaining member of the dose delivery mechanism into a second axial position with respect to the retaining member to transfer the dose delivery mechanism from the preassembled state into the assembled state.

According to an embodiment, the method comprises the step of blocking, such as irreversibly blocking, the adjusting element in the second axial position with respect to the retaining member and blocking the adjusting element from returning into the first axial position with respect to the retaining member.

The present disclosure is also directed at a method for assembling the dose delivery mechanism according to one of claims 1 to the present disclosure0, wherein the method comprises at least the following steps:
connecting the adjusting element to a retaining member of the dose delivery mechanism so that the adjusting element is in a first axial position with respect to the retaining member, wherein the adjusting element is configured to move axially from the first axial position into a second axial position with respect to the retaining member upon transfer of the dose delivery mechanism from the preassembled state into the assembled state;
providing the dose delivery mechanism in the preassembled state and with the adjusting element in the first axial position with respect to the retaining member so that the position of the piston rod is adjustable by rotating the adjusting element.

According to an embodiment, the method comprises the step of keeping the adjusting element in the first axial position until the position of the piston rod has been adjusted by rotating the adjusting element.

According to an embodiment, the step of connecting the adjusting element to the retaining member includes connecting the adjusting element to the remaining components of the dose delivery mechanism.

According to an embodiment, the method comprises the step of positioning the adjusting element in a preassembled position with respect to the housing.

According to an embodiment, the method comprises the step of biasing the adjusting element into the preassembled position with respect to the housing, for example by a biasing element, such as a spring.

According to an embodiment, the method comprises the step of positioning all remaining components of the dose delivery mechanism except for the adjusting element, the piston rod and components axially fixed to the piston rod axially in a respective zero dose position with respect to the housing, wherein the remaining components are configured to be in the respective zero dose position prior to setting a dose with the dose delivery mechanism in the assembled state.

According to an embodiment, the method comprises the step of providing a subassembly comprising a coupling member and a coupling element, wherein movement of the coupling member is restrained in the axial direction with respect to the coupling element. Furthermore, the method comprises the step of attaching the subassembly to the housing.

According to an embodiment, the subassembly further comprises an insert, wherein the insert is axially fixed to the coupling member and, for example, rotationally movable with respect to the insert.

According to an embodiment, the coupling element forms the retaining member.

In another aspect, the present disclosure is directed at a dose delivery mechanism for a medicament delivery device, the dose delivery mechanism comprising: a housing; a piston rod; and an adjusting element. The housing is configured to connect to a medicament container sealed by a plunger. Furthermore, the dose delivery mechanism has a preassembled state and an assembled state, wherein, in the assembled state, the dose delivery mechanism is configured to move the piston rod axially in a proximal direction with respect to the housing during dose delivery such that the piston rod exerts an axial force in the proximal direction on the plunger of the medicament container to expel a medicament from the medicament container. In the preassembled state, the adjusting element is configured to perform a rotation with respect to the housing, wherein the rotation of the adjusting element causes an axial movement of the piston rod for adjusting an axial position of the piston rod with respect to the housing prior to transfer of the dose delivery mechanism from the preassembled state into the assembled state. An outer rim of the adjusting element is accessible to an assembler of the device in the preassembled state to effect the rotation of the adjusting element and the axial movement of the piston rod. The adjusting element is configured to perform the rotation at least while being in a preassembled position with respect to the housing, wherein the preassembled position is a most distal position of the adjusting element with respect to the housing in the preassembled state. Furthermore, the dose delivery mechanism is configured to transfer the rotation of the adjusting element into the axial movement of the piston rod via a single threaded connection in the preassembled state, wherein the threaded connection comprises a first threaded element that is threadedly engaged with a second threaded element.

The dose delivery mechanism according to the present disclosure allows to adjust the position of the piston rod prior to assembly in a simple and user-friendly manner. By providing an adjusting element having an outer rim that is accessible to an assembler of the device, the rotation of the adjusting element can be easily effected either by directly gripping the outer rim by hand or by using an assembly tool that engages with the outer rim. In general, the adjustment of the position of the piston rod can either be performed manually or in an automated assembly line.

By allowing the rotation of the adjusting element in the preassembled position, the preassembled position being the most distal position of the adjusting element with respect to the housing, the adjustment of the position of the piston rod is further simplified since there is no need to adapt the adjustment process to, for example, an axial proximal movement of the adjusting element prior to the rotation or during the rotation.

Finally, the single threaded connection for transferring the rotation of the adjusting element into the axial movement of the piston rod provides for a simple mechanical construction of the adjusting mechanism. This results in low forces being necessary for adjustment and yet allows for precise adjustment of the position of the piston rod.

With the dose delivery mechanism, the position of the piston rod can either be adjusted to provide a gap between the plunger and a bearing of the piston rod after final assembly or it can be adjusted to position the bearing at the plunger after final assembly. The adjustment of the position of the piston rod thereby can be either performed with the medicament container already attached to the dose delivery mechanism or it can be performed prior to attaching the medicament container to the dose delivery mechanism.

The adjusting element can be configured not to move axially with respect to the housing during adjustment of the axial position of the piston rod. This provides for a mechanically simple adjustment of the piston rod.

The dose delivery mechanism can be configured to hold the adjusting element in the preassembled position. For example, the dose delivery mechanism can be configured to hold the adjusting element in the preassembled position in a way that the adjusting element autonomously takes up the preassembled position in the preassembled state. For example, the dose delivery mechanism can comprise a lock that holds the adjusting element in the preassembled position. The lock can be configured as a releasable lock, for example as a latching connection. Upon transfer of the dose delivery mechanism from the preassembled state into the assembled state, the lock can be opened to allow the adjusting element to leave the preassembled position.

The dose delivery mechanism can additionally or alternatively hold the adjusting element in the preassembled position by biasing the adjusting element into the preassembled position. The adjusting element can then be configured to be moved against the biasing force and to leave the preassembled position upon transfer of the dose delivery mechanism from the preassembled state to the assembled state.

With other embodiments, the dose delivery mechanism can not be configured to hold the adjusting element in the preassembled position and the adjusting element can be configured to axially move away from the preassembled position while the dose setting mechanism is in the preassembled state. The adjusting element can be configured to move from the preassembled position into an axial position in which the rotation with respect to the housing is no longer possible. With these embodiments, the assembler first places the adjusting element distally with respect to the housing in the preassembled position and keeps the adjusting element in the preassembled position during adjustment of the piston rod.

The medicament delivery device can be configured as an injection device, such as a pen injection device. The medicament container can be configured to receive a cannula at its proximal end to deliver the medicament through the cannula.

The dose delivery mechanism can comprise a dose definition mechanism that allows a user of the device to set at least one dose of medicament for delivery. For example, the dose definition mechanism can be configured to allow only a single predetermined dose to be set. Alternatively, the dose definition mechanism can also be configured to allow a multitude of differing predetermined doses to be set by the user, such as two or more differing doses.

The dose delivery mechanism can be configured as a single-use mechanism that allows to set a dose only once and subsequently prevents a user from setting and delivering further doses. The dose delivery mechanism can also be configured as a multi-use mechanism that allows to repeatedly set doses for delivery.

The medicament delivery device can be configured as a disposable device that is disposed of after ejecting a last dose from the medicament container.

With the dose delivery mechanism, a delivery of the medicament can require an axial force to be exerted by a user, for example on an actuation member of the dose delivery mechanism. For example, the user can have to exert the axial force with respect to the housing. The actuation member can, for example, be the adjusting member or a member rigidly connected to the adjusting member.

With other embodiments, the dose delivery mechanism can be configured as an automatic mechanism. Such a dose delivery mechanism can provide a force that is sufficient to automatically deliver the set dose from an energy reservoir, such as a spring.

In the assembled state, the dose delivery mechanism can be configured to prevent the piston rod from moving axially upon rotation of the adjusting element, unless that rotation occurs during delivery of a set dose. For example, the adjusting element can be configured to rotate with respect to the housing in the assembled state during dose setting. The dose delivery mechanism then can be configured to prevent the piston rod from moving axially upon rotation of the adjusting element during dose setting.

The first threaded element and the second threaded element can be configured to rotate with respect to each other during dose setting in the assembled state. Additionally or alternatively, the first threaded element and the second threaded element can be configured to rotate with respect to each other during dose delivery in the assembled state.

The dose delivery mechanism can comprise a bearing that is configured to directly contact the plunger of the attached medicament container. The bearing can be located at the piston rod. Thereby, it can be integrally formed with the piston rod. Alternatively, it can be configured as a separate component, like a disc, that is located in between the piston rod and the plunger. For example, the bearing can be attached to the piston rod.

The housing can be configured to connect to the medicament container by having connection means that allow attachment of a separate container holder comprising the medicament container to the housing. The connection means can be, for example, configured as a form fit, such as a snap-fit connector or a threaded connector, or as an adhesive bond, such as a welded or glued connection. Alternatively, the housing can also connect to the medicament container by comprising a container holder that is integrally formed with a housing section comprising the components of the dose delivery mechanism.

According to an embodiment, the adjusting element is configured to be rotated until a bearing located at the piston rod contacts the plunger of the medicament container. This allows to position the bearing in direct contact with the plunger. Alternatively, the bearing can also be positioned at any distance from the piston rod, for example by stopping the rotation of the adjusting element and the axial movement of the piston rod in the proximal direction prior to the bearing contacting the plunger.

According to an embodiment, the adjusting element is configured to cause proximal movement of the piston rod when being turned by the assembler. In addition, the adjusting element can also be configured to cause distal movement of the piston rod when being turned in a direction opposite to a direction causing proximal movement. This allows exact positioning of the piston rod independent of its initial position.

According to an embodiment, the adjusting element protrudes in a distal direction from the remaining members of the dose delivery mechanism in the preassembled state. The adjusting element, for example its outer rim, then is easily accessible for adjusting the piston rod. In addition, the remaining parts of the dose delivery mechanism, for example the housing, can be easily held during adjustment. For example, the adjusting element can protrude in the distal direction from a dose setting element of the dose delivery mechanism, wherein the dose setting element is configured to be gripped by a user of the device during dose setting in the assembled state.

According to an embodiment, the adjusting element is configured to be transferred from the preassembled position into an assembled position with respect to the housing when transferring the dose delivery mechanism from the preassembled state into the assembled state. For example, the adjusting element can move axially from the preassembled position into the assembled position. Thereby, the adjusting element can move in the proximal direction.

The assembled position can be a position that the adjusting element takes up in a passive state of the dose delivery mechanism, the passive state being a state in which the dose delivery mechanism is not operated by a user. For example, the assembled position can be a position that the adjusting element takes up in the assembled state prior to setting and delivering a dose of medicament. In the assembled state, the dose delivery mechanism can be configured to bias the adjusting element into the assembled position. The passive state can be a state of the dose delivery mechanism in which no dose to be delivered is set.

The transfer of the adjusting element from the preassembled position into the assembled position can cause a transfer of the dose delivery mechanism from the preassembled state into the assembled state. For example, the adjusting element can serve as a switching means to switch the dose delivery mechanism from the preassembled state into the assembled state. This allows simple final assembly of the dose delivery mechanism since a single element is first rotated to adjust the position of the piston rod and then moved to transfer the dose delivery mechanism to its assembled state.

With alternative embodiments, the adjusting element can also be configured to perform the rotation for adjusting the position of the piston rod when the adjusting element is located in an adjusting position that deviates from the preassembled position. Also with these embodiments, the dose delivery mechanism can be configured to hold the adjusting element in the preassembled position. The adjusting element then is transferred from the preassembled position into the adjusting position, for example by axially moving the adjusting element, such as by moving the adjusting element in the proximal or distal direction, prior to adjusting the position of the piston rod. This prevents unintended movement of the piston rod during final assembly by requiring a separate movement to initiate adjustment of the piston rod. The adjusting element can be configured to effect the rotation while being simultaneously forced into the adjusting position by the assembler of the device.

With these alternative embodiments, the adjusting element can be configured to be pushed in the proximal direction from the preassembled position into the adjusting position. This allows to ergonomically adjust the position of the piston rod by performing a combined push and turn operation of the adjusting element.

According to an embodiment, the dose delivery mechanism comprises a biasing element that biases the adjusting element into the preassembled position with respect to the housing in the preassembled state. The adjusting element then is releasably held in the preassembled position by the biasing force.

According to an embodiment, the adjusting element is configured to move axially from the preassembled position into the assembled position upon transferring the dose delivery mechanism from the preassembled state to the assembled state. For example, the adjusting element can be configured to move axially in the proximal direction. This allows for easy transfer of the dose delivery mechanism from the preassembled state to the assembled state.

According to an embodiment, the adjusting element is blocked, such as irreversibly blocked, in the assembled position from returning into the preassembled position with respect to the housing. By preventing the adjusting element from returning into the preassembled position, any loss of adjustment of the position of the piston rod is prevented after final assembly. While being prevented from returning into the preassembled position, the adjusting element can still be movable, such as axially movable in the assembled position. For example, the adjusting element can still be movable in a same axial direction in which the adjusting element moves upon transfer from the preassembled position into the assembled position.

According to an embodiment, the dose delivery mechanism comprises a latching mechanism that is configured to prevent the adjusting element from moving from the assembled position into the preassembled position. Such a latching mechanism provides a mechanically simple locking mechanism.

According to an embodiment, the dose delivery mechanism comprises a counter member and the latching mechanism comprises a latch part of the adjusting element and a latch counterpart of the counter member, wherein the latch part and the latch counterpart are configured to directly engage with each other in the assembled state to block movement of the adjusting element with respect to the housing at least in one direction. By providing the latch part directly at the adjusting element, the adjusting element is reliably held in the assembled state. The latch part can be integrally formed with the adjusting element, for example it can be integrally formed with the outer rim of the adjusting element.

With other embodiments, the latch part can also be part of an intermediate member that is fixedly connected to the adjusting element at least in one of an axial direction and a radial direction. For example, the intermediate member can be fixed to the adjusting element only in the axial direction or in both the axial and radial direction.

According to an embodiment, the dose delivery mechanism comprises a further latching mechanism that is configured to prevent detachment of the adjusting element from the housing in the preassembled state. Such a latching mechanism provides a well-defined configuration of the dose delivery mechanism in the preassembled state and thus facilitates adjustment of the piston rod. The latching mechanism can also be configured to hold the adjusting element in the preassembled position.

According to an embodiment, the further latching mechanism comprises a further latch part of the adjusting element and a further latch counterpart of a further counter member, wherein the further latch part and the further latch counterpart are configured to directly engage with each other in the preassembled state to block movement of the adjusting element with respect to the housing at least in one direction. By providing the further latch part directly at the adjusting element, the adjusting element is reliably prevented from detachment from the housing in the preassembled state. The further latch part can be integrally formed with the adjusting element, for example it can be integrally formed with the outer rim of the adjusting element.

With other embodiments, the further latch part can also be part of a further intermediate member that is fixedly connected to the adjusting element at least in one of an axial direction and a radial direction. For example, the further intermediate member can be fixed to the adjusting element only in the axial direction or in both the axial and radial direction.

The counter member of the latching mechanism and the further counter member of the further latching mechanism can be a single member of the dose delivery mechanism. Likewise, the intermediate member of the latching mechanism and the further intermediate member of the further latching mechanism can be a single member of the dose delivery mechanism. This provides a simple configuration of the dose delivery mechanism.

According to an embodiment, the latching mechanism and the further latching mechanism share a single latch part or a single latch counterpart. The single latch part can then be configured to engage with a first latch counterpart in the assembled state and to engage with a second latch counterpart in the preassembled state. Likewise, the single latch counterpart can be configured to engage with a first latch part in the assembled state and to engage with a second latch part in the preassembled state. Such shared latch parts or latch counterparts allow for a simple construction of the dose delivery mechanism.

According to an embodiment, a pitch of the single threaded connection defines a ratio of an axial distance to a circumferential distance and the piston rod travels the axial distance with respect to the housing upon rotation of the adjusting element by the circumferential distance in the preassembled state. The pitch of the threaded connection thus defines the gearing ratio for adjusting the axial position of the piston rod.

According to an embodiment, the first and second threaded elements rotate with respect to each other during one of dose setting and dose delivery in the assembled state, wherein the first and second threaded elements do not rotate with respect to each other during the other one of dose setting and dose delivery in the assembled state. Such a construction allows to use a threaded connection that is also active during dose delivery or dose setting for adjusting the position of the piston rod. This provides a simple construction of the dose delivery mechanism.

The second threaded element can, for example, be configured to rotate with respect to the first threaded element during dose setting and not to rotate with respect to the first threaded element during dose delivery. The second threaded element can also be configured not to rotate with respect to the first threaded element during dose setting and to rotate with respect to the first threaded element during dose delivery.

According to an embodiment, during dose delivery or dose setting in the assembled state, the first threaded element is rotationally stationary with respect to a third element of the dose delivery mechanism and the second threaded element is rotated with respect to the third element. Furthermore, during adjustment of the piston rod in the preassembled state, the first threaded element is rotated with respect to the third element of the dose delivery mechanism and the second threaded element is rotationally stationary with respect to the third element. With such a dose delivery mechanism, adjustment of the piston rod in the preassembled state is based on a kinematic reversal of the relative rotation between the first threaded element and the second threaded element during dose delivery or dose setting. Thus, the first and second threaded element can also be used for axially moving the piston rod during dose delivery. This provides for a simple construction of the dose delivery mechanism.

According to an embodiment, during dose setting or dose delivery in the assembled state, the first threaded element is axially stationary with respect to a third element of the dose delivery mechanism and the second threaded element is axially moved with respect to the third element. During adjustment of the piston rod in the preassembled state, the first threaded element is axially moved with respect to the third element of the dose delivery mechanism and the second threaded element is axially stationary with respect to the third element. With such a dose delivery mechanism, adjustment of the piston rod in the preassembled state is based on a kinematic reversal of the relative axial movement between the first threaded element and the second threaded element during dose setting or dose delivery. Thus, the first and second threaded element can also be used for axially moving the piston rod during dose delivery. This provides for a simple construction of the dose delivery mechanism.

According to an embodiment, the third element is the housing. Thus, only one of the first and second threaded elements rotates with respect to the housing during dose setting and only the other one of the first and second threaded elements rotates with respect to the housing during dose delivery.

According to an embodiment, the first threaded element is the piston rod. By providing the threaded connection directly at the piston rod, the piston rod can be precisely adjusted in the preassembled state. For example, the dose delivery mechanism does not comprise any intermediate components in between the piston rod and the threaded connection that could introduce additional play during adjustment of the piston rod.

The second threaded element can be a nut that rotates with respect to the housing during dose setting and that is rotationally fixed with respect to the housing during dose de-livery. The first threaded element then can be rotationally fixed with respect to the housing during both dose setting and dose delivery. Additionally or alternatively, the first threaded element can be rotationally fixed with respect to the housing during adjustment of the piston rod in the preassembled state and the second threaded element can be configured to rotate with respect to the housing during adjustment of the piston rod in the preassembled state.

The second threaded element can also be a dose sleeve that rotates with respect to the housing during both dose setting and dose delivery. The first threaded element then can be rotationally fixed with respect to the housing during dose delivery and it can rotate with respect to the housing during dose setting. For example, the first threaded element can be rotationally fixed with respect to the second threaded element during dose setting. In addition, the second threaded element can be configured to not rotate with respect to the housing in the preassembled state and the first threaded element can rotate with respect to the housing in the preassembled state upon rotation of the adjusting element.

According to an embodiment, one of the first threaded element and the second threaded element retains its axial position with respect to the housing during the axial movement of the piston rod in the preassembled state. That element then defines an anchor for axial movement of the piston rod in the preassembled state. This allows for precise adjustment of the piston rod since any play between the axial anchor and the threaded connection is avoided.

According to an embodiment, one of the first threaded element and the second threaded element are configured to not rotate during the rotation of the adjusting element in the preassembled state. That element then defines an anchor for rotational movement of the piston rod in the preassembled state and allows for precise adjustment of the piston rod.

According to an embodiment, the threaded connection acts between the adjusting element and the piston rod. For example, the piston rod can be rotationally fixed with respect to the first threaded element and the adjusting element can be rotationally fixed with respect to the second threaded element. Thereby, the piston rod can be configured as the first threaded element and/or the adjusting element can be configured as the second threaded element. This provides a simple construction of the dose delivery mechanism.

Alternative embodiments, the threaded connection can act between the piston rod and the housing in the preassembled state. For example, the threaded connection can act between the piston rod and an intermediate member that is rotationally and/or axially fixed with respect to the housing the preassembled state.

According to an embodiment, the dose delivery mechanism comprises a nut that is threadedly connected to, for example threadedly engaged with, the piston rod, wherein, in the preassembled state, the rotation of the adjusting element causes rotation of the nut to cause the piston rod to move axially relative to the housing. The piston rod can then be the first threaded element and/or the nut can be the second threaded element.

According to an embodiment, the adjusting element is rotationally fixed to the nut and axially slidable relative to the nut. This allows to axially move the piston rod without requiring corresponding axial movement of the adjusting element.

According to an embodiment, in the assembled state, the nut is turned by the adjusting element during dose setting and performs an axial movement due to the threaded connection to the piston rod. According to an embodiment, in the assembled state, rotation of the nut causes the nut to translate axially in a distal direction along threads located on the piston rod during dose setting and to translate in the proximal direction during dose cancellation. Axial movement of the nut with respect to the piston rod then can define the axial movement of the piston rod during dose delivery and thus the amount of medicament expelled during dose delivery.

According to an embodiment, in the assembled state, the nut does not rotate during dose delivery, moving only axially with the piston rod a distance in the proximal direction, wherein the distance is directly proportional to a set dose.

According to an embodiment, the dose delivery mechanism comprises a further member, wherein the adjusting element is rotationally decoupled from the further member during adjustment of the piston rod in the preassembled state and the adjusting element is rotationally coupled to the further member during dose setting in the assembled state. Furthermore, the adjusting element is rotationally decoupled from the further member during dose delivery in the assembled state. Rotationally coupling the adjusting element to the further member during dose setting can prevent the piston rod from moving axially during dose setting. The adjusting element can be rotationally decoupled from the further member by allowing the adjusting element being rotationally movable with respect to the further member. The adjusting element can be rotationally coupled to the further member by rotationally fixing the adjusting element to the further member.

The adjusting element can be permanently rotationally decoupled from the further member while the dose delivery mechanism is in the preassembled state. Alternatively, the adjusting element can also be only rotationally decoupled from the further member during adjustment of the position of the piston rod in the preassembled state. For example, if the adjusting element has to be brought to the adjusting position to adjust the position of the piston rod, the adjusting element can only be rotationally decoupled from the further member when being in the adjusting position. With such an embodiment, the adjusting element can be rotationally coupled to the further member when being in the preassembled position.

According to an embodiment, the further member is threadedly connected to the housing. This can lead to axial movement of both the further member and the adjusting element during dose setting. The dose delivery mechanism then can be configured to deliver a set dose if this axial movement is reversed during dose delivery, for example by a user of the device forcing the further member proximally in the axial direction.

According to an embodiment, the further member is a dose indication member indicating a set dose. The dose indication member can comprise markings that are visible from an exterior of the housing at least during dose setting.

According to an embodiment, the further member rotates with respect to the piston rod during dose setting and/or the further member does not rotate with respect to the piston rod during the adjustment of the position of the piston rod in the preassembled state.

According to an embodiment, the further member maintains its axial position with respect to the housing of the dose delivery mechanism upon the rotation of the adjusting element in the preassembled state.

According to an embodiment, the dose delivery mechanism comprises a clutch mechanism having a first clutch member and a second clutch member. The first clutch member and the second clutch member engage with each other to rotationally couple the adjusting element to the further member in a closed state of the clutch mechanism during dose setting in the assembled state and the first clutch member and the second clutch member disengage from each other to rotationally decouple the adjusting element from the further member in an opened state of the clutch mechanism during dose delivery in the assembled state. Such a clutch mechanism provides mechanically simple means for rotationally coupling and decoupling the adjusting element and the further member in the assembled state.

In general, the clutch mechanism is in the opened state when the first clutch member and the second clutch member do not engage with each other and the clutch is in the closed state when the first clutch member and the second clutch member engage with each other.

The clutch mechanism can connect the adjusting element to the further member via a coupling member. The coupling member can be rotationally fixed with respect to the further member. If the clutch mechanism is opened in the preassembled state, it allows a rotation of the adjusting element with respect to the coupling member, and if the clutch mechanism is opened in the assembled state during dose delivery, it allows a rotation of the adjusting element with respect to the coupling member.

According to an embodiment, the adjusting element is rotationally decoupled from both the first clutch member and the second clutch member in the preassembled state. Thus, the clutch mechanism is not rotationally connected to the adjusting member in the preassembled state. This then also rotationally decouples the adjusting element from the further member. The clutch mechanism can be in the closed state during adjustment of the piston rod in the preassembled state. For example, the clutch mechanism can be permanently in the closed state while the dose delivery mechanism is in the preassembled state.

According to an embodiment, the adjusting element is rotationally fixed with respect to one of the first clutch member and the second clutch member in the preassembled state. During adjustment of the position of the piston rod in the preassembled state, the clutch mechanism is in the opened state and the first clutch member and the second clutch member disengage from each other thus allowing a rotation of the adjusting element with respect to the further member. With this embodiment, the action of the clutch mechanism is also used to rotationally decoupled the adjusting element from the further member during adjustment of the position of the piston rod.

The clutch mechanism can be permanently in the opened state while the dose de-livery mechanism is in the preassembled state. The clutch mechanism can also be only in the opened state during adjustment of the piston rod in the preassembled state. For example, if the adjusting element has to be brought to the adjusting position for adjusting the position of the piston rod, the clutch mechanism can only be in the opened state if the adjusting element is in the adjusting position and the clutch mechanism can be in the closed state if the adjusting element is in the preassembled position.

According to an embodiment, the clutch mechanism comprises a first clutch part and a second clutch part, wherein the first clutch part and the second clutch part are engaged with each other in the closed state of the clutch mechanism and disengage from each other in the opened state of the clutch. The first clutch part thereby is located at a first axial side from the second clutch part in the opened state of the clutch mechanism in the preassembled state of the dose delivery mechanism and the first clutch part is located at a second axial side from the second clutch part in the opened state in the assembled state of the dose delivery mechanism. The second axial side is opposite the first axial side.

Such a construction provides a simple mechanism to open the clutch mechanism in the preassembled state. For example, the adjusting element and the first clutch part can axially move upon transfer of the dose delivery mechanism from the preassembled state to the assembled state and this axial movement can cause the first clutch part to engage with the second clutch part and thus close the clutch.

In general, the dose delivery mechanism can comprise a clutch mechanism having a first clutch member and a second clutch member, wherein, in the assembled state, the clutch mechanism is closed during one of dose setting and dose delivery and opened during the other one of dose setting and dose delivery. The adjusting element can then be rotationally decoupled from both the first clutch member and the second clutch member in the preassembled state.

According to an embodiment, the adjusting element is rotationally coupled, such as permanently rotationally coupled, to one of the first clutch member and the second clutch member in the assembled state. In addition, the adjusting element can be rotationally coupled, such as permanently rotationally coupled to the one of the first clutch member and the second clutch member in the preassembled state. For example, the adjusting element can constitute the one of the first clutch member and the second clutch member.

In general, the clutch mechanism can be opened in the preassembled state thus allowing a rotation of the adjusting element with respect to the further member and the clutch mechanism can be opened in the assembled state during dose delivery thus allowing a rotation of the adjusting element with respect to the further member. The clutch mechanism then can be closed in the assembled state during dose setting.

According to an embodiment, the dose delivery mechanism is configured to hold the clutch mechanism in the opened state in the preassembled state. For example, the dose delivery mechanism can bias the clutch mechanism into the opened state. The dose delivery mechanism can also hold the clutch mechanism in the opened state by locking the clutch mechanism in the opened state, for example, the dose delivery mechanism can reversibly lock the clutch mechanism in the opened state.

According to an embodiment, the clutch mechanism is only allowed to close from the opened state in the preassembled state when transferring the dose delivery mechanism from the preassembled state into the assembled state. This securely keeps the clutch mechanism opened in the preassembled state.

With alternative embodiments, the dose delivery mechanism can be configured to hold the clutch mechanism in the closed state in the preassembled state. For example, the dose delivery mechanism can bias the clutch mechanism into the closed state. The clutch mechanism then can be configured to be opened by moving the adjusting element, for example by moving the adjusting element from the preassembled position into the adjusting position.

According to an embodiment, the dose delivery mechanism comprises an actuation element and the clutch mechanism is transferred from the closed state into the opened state upon proximal movement of the actuation element from a dose setting position into a dose delivery position to effect proximal movement of the piston rod upon proximal movement of the actuation element. The actuation element can be a pushbutton that is configured to be pressed by a user to deliver a set amount of medicament. The actuation element can, for example, be formed by the adjusting element.

According to an embodiment, the adjusting element is rotationally decoupled from an additional member of the dose delivery mechanism during adjustment of the piston rod in the preassembled state and during dose setting in the assembled state. Furthermore, the adjusting element is rotationally fixed to the additional member during dose delivery in the assembled state. Rotation of the adjusting element during dose setting then can contribute to setting a dose to be delivered by the dose delivery mechanism without moving the piston rod during dose setting.

According to an embodiment, the additional member is the housing.

According to an embodiment, the adjusting element is rotatable with respect to a counter element in the preassembled state and rotationally fixed, such as irreversibly rotationally fixed, to the counter element in the assembled state. Rotational fixation of the adjusting element to the counter element then can cause the dose delivery mechanism to transfer from the preassembled state to the assembled state. The adjusting element can, for example, be additionally axially fixed, such as irreversibly axially fixed, to the counter element in the assembled state.

According to an embodiment, the adjusting element is in a first axial position with respect to the counter element of the dose delivery mechanism in the preassembled state and the adjusting element is configured to move axially from the first axial position into a second axial position with respect to the counter element upon transfer of the dose delivery mechanism from the preassembled state into the assembled state, wherein the adjusting element is axially fixed, such as irreversibly axially fixed, to the counter element in the assembled state. The adjusting element and the counter element than can act as a single member of the dose delivery mechanism in the assembled state.

According to an embodiment, the dose delivery mechanism comprises a latching mechanism that acts between the adjusting element and the counter element, wherein the latching mechanism is configured to block movement of the adjusting element from the second position into the first position in the assembled state. This prevents the dose delivery mechanism from returning to the preassembled state of the final assembly.

The dose delivery mechanism can comprise an intermediate element that acts in between the adjusting element and the counter element. The latching mechanism then can act between the intermediate element and one of the adjusting element and the counter element. The other one of the adjusting element in the counter element then can be axially and/or rotationally fixed to the intermediate element. For example, the latching mechanism can act between the adjusting element and the intermediate element and the intermediate element can be rotationally and/or axially fixed to the counter element.

A latch part of the latching mechanism can be formed at the adjusting element. Additionally or alternatively, a further latch part of the latching mechanism can be formed at the counter element.

According to an embodiment, the counter element is a dose setting element of the dose delivery mechanism, wherein the dose setting element is configured to be gripped by the user of the dose delivery mechanism in the assembled state to set a dose to be delivered. The adjusting element and the counter element then can provide a dose setting member of the dose delivery mechanism that is configured to be rotated by a user to set a dose to be delivered in the assembled state. This allows to integrate the fixation of the adjusting element to the counter element in a mechanical simple way into the dose setting element.

According to an embodiment, the adjusting element protrudes distally from the counter element in the preassembled state. Such a configuration facilitates manipulation of the adjusting element in the preassembled state.

According to an embodiment, the adjusting element does not protrude distally from the counter element in the assembled state. This prevents the adjusting element from being easily accessible to a user of the device and thus prevents a user forcing the adjusting element into the preassembled position again.

According to an embodiment, the dose delivery mechanism comprises a rotational lock, wherein the rotational lock allows rotational movement between the adjusting element and the counter element in the preassembled state of the dose delivery mechanism. The adjusting element is rotationally fixed with respect to the counter element in the assembled state via the rotational lock and the rotational lock allows fixation of the adjusting element to the counter element in a multitude of mutual relative rotational positions. Such a rotational lock provides a reliable rotational fixation of the adjusting element to the counter element irrespective of the amount of rotation needed to position the piston rod in the preassembled state.

The rotational lock can act between the intermediate element and one of the adjusting element and the counter element. The other one of the adjusting element and the counter element then can be rotationally fixed to the intermediate element, for example both in the preassembled state and the assembled state. A part of the rotational lock can be formed at, such as integrally formed with, the adjusting element. Additionally or alternatively, a further part of the rotational lock can be formed at, such as integrally formed with, the counter element.

According to an embodiment, the rotational lock comprises a toothed part defining the multitude of rotational positions and an engaging part that is configured to engage with the toothed part upon transfer of the dose delivery mechanism from the preassembled state into the assembled state to rotationally lock the adjusting element to the counter element.

The toothed part can comprise a multitude of teeth that are circumferentially arranged around a longitudinal axis of the dose delivery mechanism. The teeth can, for example, be located at the intermediate element. The engaging part can comprise at least one engaging tooth that engages with the teeth of the toothed part can rotationally lock the adjusting element to the counter element. For example, be engaging part can comprise a multitude of engaging teeth. This provides a reliable rotational fixation.

According to an embodiment, the dose delivery mechanism comprises an axial lock, wherein the axial lock allows axial movement between the adjusting element and the counter element in the preassembled state of the dose delivery mechanism and prevents axial movement between the adjusting element and the counter element in the assembled state.

The axial lock can act between the intermediate element and one of the adjusting element and the counter element. The other one of the adjusting element and the counter element then can be rotationally fixed to the intermediate element, for example both in the preassembled state and the assembled state. A part of the axial lock can be formed at, such as integrally formed with, the adjusting element. Additionally or alternatively, a further part of the axial lock can be formed at, such as integrally formed with, the counter element.

According to an embodiment, the axial lock allows axial fixation of the adjusting element to the counter element in a multitude of mutual relative rotational positions. For example, the axial lock can comprise a circumferential part, such as a circumferential rib, that is located circumferentially around a longitudinal axis of the dose delivery mechanism. Furthermore, the axial lock can comprise at least one engaging part that is configured to engage with the circumferential part upon axially locking the adjusting element to the counter element.

The axial lock can be configured as a snap fit connection. For example, the engaging part can be configured as a flexible hook that snaps onto the circumferential part when closing the axial lock.

According to an embodiment, the dose delivery mechanism comprises a connector, wherein the adjusting element is rotationally and/or axially fixed to the counter element in the assembled state via the connector. Such a connector facilitates implementation of the rotational lock and/or the axial lock. The connector can constitute the intermediate element.

According to an embodiment, the connector is axially locked to the counter element both in the preassembled state and in the assembled state. The axial lock between the adjusting element and the counter element then can act between the adjusting element and the connector.

According to an embodiment, the connector is rotationally locked to the counter element both in the preassembled state and in the assembled state. This provides secure rotational locking of the adjusting element to the counter element via the connector.

According to an embodiment, the rotational lock is located between the adjusting element and the connector. For example, a first part of the rotational lock can be formed at the adjusting element and a second part of the rotational lock can be formed at the connector.

According to an embodiment, the connector comprises one of the toothed part and the engaging part and the adjusting element comprises the other one of the toothed part and the engaging part. For example, the engaging part can be formed at the adjusting element and the toothed part can be formed at the connector.

According to an embodiment, the axial lock is located between the adjusting element and the connector. For example, a first part of the axial lock, such as the engaging part, can be formed at the adjusting element and a second part of the axial lock, such as the circumferential part, can be formed at the connector.

According to an embodiment, the adjusting element is configured to rotate with respect to the piston rod upon the rotation with respect to the housing in the preassembled state. This allows to provide the threaded connection that transfers the rotation of the adjusting element into the axial movement of the piston rod in between the adjusting element and the piston rod.

According to an embodiment, the piston rod is rotationally fixed with respect to the housing in the preassembled state.

According to an embodiment, the adjusting element is rotationally fixed with respect to the piston rod in the preassembled state. This allows to rotate the piston rod during adjustment of its axial position. The threaded connection then can act between the piston rod and the housing.

According to an embodiment, the piston rod is rotationally fixed with respect to the housing in the assembled state during dose setting and/or during dose delivery. For example, the piston rod can be permanently fixed with respect to the housing.

According to an embodiment, the adjusting element is in a first axial position with respect to a retaining member of the dose delivery mechanism in the preassembled state, wherein the adjusting element is configured to move axially from the first axial position into a second axial position with respect to the retaining member upon transfer of the dose delivery mechanism from the preassembled state into the assembled state, wherein the adjusting element is rotatable with respect to the retaining member in the preassembled state.

The adjusting element can be in the first axial position with respect to the retaining member when it is in the preassembled position with respect to the housing and it can be in the second axial position with respect to the retaining member when it is in the assembled position with respect to the housing. The retaining member can be an extension that connects the adjusting element to the housing. The retaining member can protrude distally from the housing. The retaining member can be rotationally fixed and axially movable with respect to the housing. The retaining member can be configured as a housing extension and at least parts of the retaining member can form an outer shell of the dose delivery mechanism.

The retaining member can be configured as a dose selector or as a sleeve or as a coupling element of the dose delivery mechanism.

According to an embodiment, the adjusting element is rotatable with respect to the retaining member during dose setting in the assembled state. Rotation of the adjusting element with respect to the retaining member then can be both used to adjust the piston rod in the preassembled state and to set a dose in the assembled state.

According to an embodiment, the adjusting element is rotationally fixed to the retaining member during dose delivery in the assembled state. This can also rotationally fix the adjusting element to the housing via the retaining member. Rotationally fixing the adjusting element to the retaining member can prevent altering a set dose during dose delivery.

According to an embodiment, the adjusting element is axially movable with respect to the retaining member in the assembled state. For example, the adjusting element can be axially movable to transfer the dose delivery mechanism from a dose setting state to a dose delivery state in the assembled state.

With alternative embodiments, the adjusting element can also be axially fixed with respect to the retaining member in the assembled state. With these embodiments, the adjusting element can be configured to axially move together with the retaining member to transfer the dose delivery mechanism from the dose setting state to the dose delivery state.

According to an embodiment, the adjusting element is biased in a distal direction when the dose delivery mechanism is in the preassembled state. This can provide a simple mechanism for holding the adjusting element in the preassembled position.

According to an embodiment, the adjusting element is configured to take up a dose setting position in the assembled state and the adjusting element is movable, for example axially movable, in the assembled state. The adjusting element can take up the dose setting position with respect to a second clutch member of a clutch mechanism of the dose de-livery mechanism. With the clutch mechanism can be in one of an opened state or a closed state if the adjusting element takes up the dose setting position.

The adjusting element also can take up the dose setting position with respect to the housing and can be movable with respect to the housing. Alternatively, the adjusting element can take up the dose setting position with respect to the retaining member and it can be movable with respect to the retaining member.

According to an embodiment, the adjusting element is configured to move proximally from the dose setting position into a dose delivery position in the assembled state. This axial movement can then change the dose delivery mechanism from a dose setting state, in which a user can set a dose to be delivered, to a dose delivery state, in which a user can deliver the set dose. The adjusting element can move into the dose delivery position with respect to the second clutch member. The clutch mechanism can be in the other one of the opened state in the closed state if the adjusting element is in the dose delivery position.

According to an embodiment, the adjusting element is biased into the dose setting position in the assembled state. This allows for simple dose setting without the need to first manually position the adjusting element in the dose setting position.

According to an embodiment, the adjusting element is configured as a dose setting element of the dose delivery mechanism, wherein the dose setting element is configured to be gripped by the user of the dose delivery mechanism to set a dose to be delivered in the assembled state.

According to an embodiment, the adjusting element is configured to rotate in the assembled state to set a dose of the medicament to be delivered by the dose delivery mechanism. Rotation of the adjusting element can thus serve to adjust the position of the piston rod in the preassembled state and to set a dose without moving the piston rod in the assembled state. This provides a mechanical simple construction of the dose delivery mechanism.

According to an embodiment, the dose delivery mechanism comprises a locking mechanism, wherein the locking mechanism rotationally locks the adjusting element to the housing during dose delivery in the assembled state.

According to an embodiment, the dose delivery mechanism comprises a dose definition mechanism that defines rotational positions of the adjusting element with respect to the housing that correspond to doses settable by the user in the assembled state, wherein the dose definition mechanism is not active during the rotation of the adjusting element in the preassembled state. This allows for exact positioning of the piston rod by rotating the adjusting element without interference of the dose definition mechanism.

According to an embodiment, the dose definition mechanism comprises at least one engagement feature and at least one dose stop that rotate with respect to each other upon rotation of the adjusting element in the assembled state and that engage with each other upon setting a dose in the assembled state.

According to an embodiment, the engagement feature and the at least one dose stop do not rotate with respect to each other during the rotation of the adjusting element in the preassembled state. This prevents the dose definition mechanism from being active during a rotation of the adjusting element in the preassembled state.

According to an embodiment, the engagement feature and the at least one dose stop rotate with respect to each other during the rotation of the adjusting element in the preassembled state and the engagement feature and the at least one dose stop do not engage with each other in the preassembled state. For example, the engagement feature and the at least one dose stop can be located axially offset from each other in the preassembled state and/or during adjustment of the position of the piston rod. This also prevents the dose definition mechanism from being active during a rotation of the adjusting element in the preassembled state.

According to an embodiment, the housing comprises a connector for connecting the medicament container to the housing and the connector is configured to connect the medicament container axially movable to the housing so that the medicament container is configured to perform an axial movement from a receiving position into an operating position after connection to the housing.

The bearing of the piston rod thereby can be located at a distance from the plunger within the medicament container when the medicament container is in the receiving position and the bearing can be in contact with the plunger when the medicament container is in the operating position.

The connector can be configured as a threaded connector and the medicament container can be configured to move from the receiving position into the operating position by screwing a container holder comprising the medicament container along the threaded connector.

According to an embodiment, the connector is configured to bring the plunger into contact with a bearing located at the piston rod upon the axial movement of the medicament container from the receiving position into the operating position. For example, the connector can be configured to bring the plunger into contact with the bearing before the medicament container reaches the operating position. This allows the bearing to push upon the plunger during the movement of the medicament container, for example to expel the amount of medicament and/or to reconstitute a lyophilized medicament.

Movement of the medicament container from the receiving position into the operating position thereby can cause an axial movement of the plunger within the medicament container due to the bearing pushing on the plunger. This then can also involve expelling an amount of medicament. The movement of the medicament container from the receiving position into the operating position can be performed by a user prior to expelling a first dose of medicament.

Additionally or alternatively, the axial movement of the plunger within the medicament container can cause a medicament within the medicament container to reconstitute from a lyophilized state into a solution.

With embodiments, in which the medicament container moves from the receiving position into the operating position, the position of the piston rod can be adjusted in the preassembled state in a way that the bearing contacts the piston rod during the movement of the medicament container. Additionally, the piston rod can be adjusted to a position in which the amount of medicament is expelled at the end of the movement of the medicament container. The adjusted position of the piston rod can cause the contact between the bearing and the plunger and/or the ejection of the medicament regardless of an initial position of the plunger with respect to a housing of the cartridge as long as the initial position is within predetermined manufacturing tolerances.

With other embodiments, the position of the piston rod can also be adjusted in a way that the bearing of the piston rod contacts the plunger in the assembled state.

The present disclosure is also directed at a medicament delivery device having a dose delivery mechanism according to the present disclosure and a medicament container attached to the dose delivery mechanism. The medicament container comprises a plunger and a bearing located at the piston rod is positioned at a predetermined distance with respect to the plunger.

All embodiments and technical effects that are disclosed in connection with the dose delivery mechanism or the method according to the present disclosure also apply to the medicament delivery device and vice versa.

According to an embodiment, the predetermined distance is zero so that the bearing contacts the plunger. This allows a user of the medicament delivery device to accurately expel also the first dose of medicament without requiring a priming of the device prior to use.

According to an embodiment, the predetermined distance is larger than zero. This prevents the piston rod from pressurizing the plunger prior to use of the device, for example during transport.

According to an embodiment, the predetermined distance is smaller than an axial distance the medicament container is travelling from the receiving position into the operating position. The plunger thus can be brought into contact with the bearing upon movement of the medicament container from the receiving position into the operating position.

The present disclosure is further directed at a method for adjusting a position of a piston rod of a dose delivery mechanism for a medicament delivery device, the dose delivery mechanism comprising: a housing; a piston rod; and an adjusting element. The housing thereby is configured to connect to a medicament container sealed by a plunger. Furthermore, the method comprises:
providing the dose delivery mechanism in a preassembled state, wherein an outer rim of the adjusting element is accessible to an assembler of the device in the preassembled state to effect rotation of the adjusting element and axial movement of the piston rod;
adjusting, in the preassembled state, an axial position of the piston rod with respect to the housing by rotating the adjusting element and thereby causing an axial movement of the piston rod with respect to the housing, wherein the rotation of the adjusting element is transferred into the axial movement of the piston rod via a single threaded connection of the dose delivery mechanism; and
transferring the dose delivery mechanism from the preassembled state into an assembled state, wherein, in the assembled state, the dose delivery mechanism is configured to move the piston rod axially in a proximal direction with respect to the housing during dose delivery such that the piston rod exerts an axial force in the proximal direction on the plunger of the medicament container to expel a medicament from the medicament container.

The dose delivery mechanism can be the dose delivery mechanism according to the present disclosure. All embodiments and technical effects that are disclosed in connection with the dose delivery mechanism also apply to the method and vice versa. Additionally or alternatively, the medicament delivery device can be the medicament delivery device according to the present disclosure. All embodiments and technical effects that are disclosed in connection with the medicament delivery device also apply to the method and vice versa.

According to an embodiment, the dose delivery mechanism is provided in the preassembled state with the medicament container attached. The method then can comprise a step of placing a bearing located at the piston rod at a predetermined distance from the plunger of the medicament container. For example, the predetermined distance can be zero so that the bearing contacts the plunger. The predetermined distance also can be larger than zero so that the bearing is located away from the plunger.

The method can also comprise a step of determining the distance between the bearing and the plunger, for example a step of monitoring the distance between the bearing on the plunger. These steps, can, for example, be performed by visually inspecting the position of the bearing, for example through a transparent component of the medicament delivery device. The distance can also be determined by sensing a contact between the bearing and the plunger, for example, by sensing an increase in torque needed to rotate the adjusting element after the bearing has contacted the plunger.

According to an embodiment, the medicament container does not move with respect to the housing upon adjusting the axial position of the piston rod. For example, the medicament container can be fixedly connected to the housing of the dose delivery mechanism in the preassembled state.

According to an embodiment, the axial position of the piston rod is adjusted to place a bearing located at the piston rod in contact with a reference surface. Such a reference surface provides a simple way of adjusting the position of the piston rod to a predetermined axial position.

According to an embodiment, the reference surface is provided by a surface of the plunger of the medicament container. This provides exact positioning of the piston rod with respect to the plunger of the medicament container actually used with the medicament delivery device.

According to an embodiment, the dose delivery mechanism is provided in the preassembled state without the medicament container attached and the method further comprises placing the dose delivery mechanism in an assembly jig.

Such an assembly jig helps to position the piston rod at a well-defined position with respect to the housing. For example, at least parts of the assembly jig or the assembly jig can be axially fixed with respect to the housing after having placed the dose delivery mechanism in the assembly jig.

According to an embodiment, the reference surface is provided by a surface of the assembly jig. This allows to adjust the piston rod to a well-defined position with respect to the housing without the need of attaching the medicament container and/or without the bearing having to contact the plunger of the medicament container.

According to an embodiment, the piston rod is axially moved during the adjusting of the axial position until the rotation of the adjusting element requires a predetermined torque. The torque can be monitored by a measurement device during adjustment of the position of the piston rod. Increase of the torque can be caused by the bearing of the piston rod touching the reference surface, such as the surface of the plunger of the medicament container attached to the housing or the reference surface of the assembly jig to which the dose delivery mechanism has been attached.

According to an embodiment, the method further comprises measuring a position of the piston rod with a measurement device for determining an adjusted position of the piston rod. The measurement device can be one of an optical measurement device and a mechanical measurement device, such as an assembly jig having a reference surface. The optical measurement device can be a camera or the like. With the optical measurement device, the position of the piston rod can be measured through a transparent part of the medicament de-livery device, for example through a transparent medicament container holder and/or a transparent medicament container.

### BRIEF DESCRIPTION OF THE DRAWINGS

Exemplary embodiments and functions of the present disclosure are described herein in conjunction with the following drawings.

FIG. 1 shows an exploded view of an injection pen according to the disclosure.

FIG. 2A shows a perspective view of a knob cover of the injection pen of FIG. 1.

FIG. 2B shows a side view of the knob cover of FIG. 2A.

FIG. 2C shows a section view of the knob cover of FIG. 2B along the line A-A of FIG. 2B.

FIG. 3A shows a perspective view of an injection button of the injection pen of FIG. 1.

FIG. 3B shows a side view of the injection button of FIG. 3A.

FIG. 3C shows a section view of the injection button of FIG. 3B along the line A-A of FIG. 3B.

FIG. 3D shows a section view of the injection button of FIG. 3B along the line B-B of FIG. 3B.

FIG. 3E shows a section view of the injection button of FIG. 3B along the line C-C of FIG. 3B.

FIG. 4A shows a perspective view of a snap ring of the injection pen of FIG. 1.

FIG. 4B shows a top view of the snap ring of FIG. 4A.

FIG. 4C shows a side view of the snap ring of FIG. 4A.

FIG. 4D shows a bottom view of the snap ring of FIG. 4A.

FIG. 5A shows a first perspective view of a dose setting knob of the injection pen of FIG. 1.

FIG. 5B shows a second perspective view of the dose setting knob of FIG. 5A.

FIG. 5C shows a side view of the dose setting knob of FIG. 5A.

FIG. 5D shows a section view of the dose setting knob of FIG. 5A along line A-A of FIG. 5C.

FIG. 6A shows a perspective view of a snap element of the injection pen of FIG. 1.

FIG. 6B shows a side view of the snap element of FIG. 6A.

FIG. 6C shows a section view of the snap element of FIG. 6A along the line A-A of FIG. 6B.

FIG. 7A shows a perspective view of a connector of the injection pen of FIG. 1.

FIG. 7B shows a bottom view of the connector of FIG. 7A.

FIG. 7C shows a side view of the connector of FIG. 7A.

FIG. 7D shows a top view of the connector of FIG. 7A.

FIG. 8A shows a first perspective view of a dose selector of the injection pen of FIG. 1.

FIG. 8B shows a bottom view of the dose selector of FIG. 8A.

FIG. 8C shows a side view of the dose selector of FIG. 8A.

FIG. 8D shows a section view of the dose selector of FIG. 8A along the line A-A of FIG. 8C.

FIG. 8E shows a section view of the dose selector of FIG. 8A along the line B-B of FIG. 8C.

FIG. 9 shows a second perspective view of the dose selector of FIG. 8A.

FIG. 10A shows a perspective view of a knob key of the injection pen of FIG. 1.

FIG. 10B shows a side view of the knob key of FIG. 10A.

FIG. 11A shows a perspective view of a housing of the injection pen of FIG. 1.

FIG. 11B shows a side view of the housing of FIG. 11A.

FIG. 11C shows a section view of the housing of FIG. 11A along the line A-A of FIG. 11B.

FIG. 12A shows a first side view of a dose setting sleeve of the injection pen of FIG. 1.

FIG. 12B shows a second side view of the dose setting sleeve of FIG. 12A.

FIG. 12C shows a third side view of the dose setting sleeve of FIG. 12A.

FIG. 12D shows a fourth side view of the dose setting sleeve of FIG. 12A.

FIG. 12E shows a front view of the dose setting sleeve of FIG. 12A.

FIG. 12F shows a first perspective view of the dose setting sleeve of FIG. 12A.

FIG. 12G shows a second perspective view of the dose setting sleeve of FIG. 12A.

FIG. 13A shows a perspective view of a driver of the injection pen of FIG. 1.

FIG. 13B shows a first side view of the driver of FIG. 13A.

FIG. 13C shows a second side view of the driver of FIG. 13A.

FIG. 13D shows a section view of the driver of FIG. 13A along the line A-A of FIG. 13C.

FIG. 14A shows a first perspective view of a nut of the injection pen of FIG. 1.

FIG. 14B shows a second perspective view of the nut of FIG. 14A.

FIG. 14C shows a side view of the nut of FIG. 14A.

FIG. 14D shows a first section view of the nut of FIG. 14A along the line A-A of FIG. 14C.

FIG. 14E shows a second section view of the nut of FIG. 14A along the line B-B of FIG. 14C.

FIG. 15A shows a first side view of a piston rod guide of the injection pen of FIG. 1.

FIG. 15B shows a second side view of the piston rod guide of FIG. 15A.

FIG. 15C shows a section view of the piston rod guide of FIG. 15A along the line A-A of FIG. 15A.

FIG. 15D shows a perspective view of the piston rod guide of FIG. 15A.

FIG. 16A shows a first longitudinal section view of the piston rod guide of FIG. 15A.

FIG. 16B shows a second longitudinal section view of the piston rod guide of FIG. 15A.

FIG. 16C shows a perspective view of the piston rod guide of FIG. 15A.

FIG. 17A shows a side view of a piston rod of the injection pen of FIG. 1.

FIG. 17B shows a section view of the piston rod of FIG. 17A along the line A-A of FIG. 17A.

FIG. 17C shows a first perspective view of the piston rod of FIG. 17A.

FIG. 17D shows a second perspective view of the piston rod of FIG. 17A.

FIG. 18A shows a perspective view of a piston disc of the injection pen of FIG. 1.

FIG. 18B shows a top view of the piston disc of FIG. 18A.

FIG. 18C shows a section view of the piston disc of FIG. 18A along the line A-A of FIG. 18B.

FIG. 19A shows a perspective view of a dual chamber cartridge of the injection pen of FIG. 1.

FIG. 19B shows a side view of the dual chamber cartridge of FIG. 19A.

FIG. 19C shows a section view of the dual chamber cartridge of FIG. 19A along the line A-A of FIG. 19B.

FIG. 20A shows a perspective view of a cartridge container of the injection pen of FIG. 1.

FIG. 20B shows a first side view of the cartridge container of FIG. 20A.

FIG. 20C shows a second side view of the cartridge container of FIG. 20A.

FIG. 20D shows a section view of the cartridge container of FIG. 20A along the line A-A of FIG. 20C FIG. 21A shows a first perspective view of a cartridge key of the injection pen of FIG. 1.

FIG. 21B shows a second perspective view of the cartridge key of FIG. 21A.

FIG. 21C shows a first side view of the cartridge key of FIG. 21A.

FIG. 21D shows a second side view of the cartridge key of FIG. 21A.

FIG. 22A shows a third side view of the cartridge key of FIG. 21A.

FIG. 22B shows a section view of the cartridge key of FIG. 21A along the line A-A of FIG. 22A.

FIG. 23A shows a side view of the injection pen of FIG. 1 in an as-delivered state.

FIG. 23B shows a section view of the injection pen of FIG. 23A along the line A-A of FIG. 23A.

FIG. 24 shows a perspective view of the injection pen of FIG. 23A without the knob cover and with some parts displayed transparently.

FIG. 25A shows a second side view of the injection pen of FIG. 23A.

FIG. 25B shows a section view of the injection pen of FIG. 23A along the line A-A of FIG. 25A.

FIG. 26A shows a side view of the injection pen of FIG. 1 in a reconstitution state.

FIG. 26B shows a section view of the injection pen of FIG. 26A along the line A-A of FIG. 26A.

FIG. 27A shows a second side view of the injection pen of FIG. 26A.

FIG. 27B shows a third side view of the injection pen of FIG. 26A.

FIG. 27C shows a section view of the injection pen of FIG. 26A along the line A-A of FIG. 27B.

FIG. 28A shows a side view of the injection pen of FIG. 1 in a knob cover unfastening state.

FIG. 28B shows a section view of the injection pen of FIG. 28A along the line A-A of FIG. 28A.

FIG. 29A shows a side view of the injection pen of FIG. 1 in an end of reconstitution state.

FIG. 29B shows a section view of the injection pen of FIG. 29A along the line A-A of FIG. 29A.

FIG. 30A shows a side view of the injection pen of FIG. 1 in a set dose state.

FIG. 30B shows a section view of the injection pen of FIG. 30A along the line A-A of FIG. 30A.

FIG. 31A shows a side view of the injection pen of FIG. 1 in a start of injection state.

FIG. 31B shows a section view of the injection pen of FIG. 31A along the line A-A of FIG. 31A.

FIG. 32A shows a further side view of the injection pen of FIG. 1 in a start of injection state.

FIG. 32B shows an enlarged section view of the injection pen of FIG. 32A along the line A-A of FIG. 32A.

FIG. 33A shows a side view of the injection pen of FIG. 1 in an end of injection state.

FIG. 33B shows a section view of the injection pen of FIG. 33A along the line A-A of FIG. 33A.

FIG. 34 shows a second injection pen according to the present disclosure in a preassembled state.

FIG. 35 shows another view of the second injection pen in the preassembled state.

FIG. 36 shows an exploded view of the second injection pen.

FIG. 37 shows a longitudinal cut through the second injection pen in the preassembled state.

FIG. 38 shows a detailed view of a distal portion of the second injection pen in the preassembled state.

FIG. 39 shows a detailed view of the distal end of the second injection pen in an assembled state during dose setting.

FIG. 40 shows another view of the distal end of the second injection pen in the assembled state during dose setting.

FIG. 41 shows a detailed view of the distal end of the second injection pen in an assembled state during dose delivery.

FIG. 42 shows another view of the distal end of the second injection pen and the assembled state during dose delivery.

FIG. 43 shows a longitudinal cut through the dose setting element of the first and second injection pen.

FIG. 44 shows a perspective view of the longitudinal cut through the dose setting element.

FIG. 45 shows a perspective distal view of the dose setting element.

FIG. 46 shows a prospective proximal view of the dose setting element.

FIG. 47 shows a perspective view of a third injection pen according to the present disclosure.

FIG. 48 shows an exploded view of the third injection pen.

FIG. 49 shows a longitudinal cut through a dose delivery mechanism of the third injection pen.

FIG. 50 shows a perspective distal view of a dosing member of the third injection pen.

FIG. 51 shows a perspective longitudinal cut through the dosing member of the third injection pen.

FIG. 52 shows a perspective view of a piston rod of the third injection pen.

FIG. 53 shows a perspective distal view of an extension of the third injection pen

FIG. 54 shows a distal view of the extension shown in FIG. 53.

FIG. 55 shows a proximal view of the extension shown in FIG. 53.

FIG. 56 shows a perspective view of a coupling element of the third injection pen.

FIG. 57 shows a perspective view of a bearing, the piston rod, the extension, the coupling element, and an adjusting element of the third injection pen.

FIG. 58 shows a proximal perspective view of the adjusting element of the third injection pen.

FIG. 59 shows a side view of the adjusting element of the third injection pen.

FIG. 60 shows a radial cut through the adjusting element of the third injection along the line A-A in FIG. 59.

FIG. 61 shows a radial cut through the adjusting element of the third injection along the line B-B in FIG. 59.

FIG. 62 shows a perspective view of a coupling member of the third injection pen.

FIG. 63 shows a perspective distal view of a sleeve of the third injection pen.

FIG. 64 shows a longitudinal cut through the sleeve of the third injection pen.

FIG. 65 shows a perspective view of a housing insert of the third injection pen.

FIG. 66 shows a perspective view of a connector of the third injection pen.

FIG. 67 shows a perspective view of a longitudinal cut through the connector of the third injection pen.

FIG. 68 shows a side view of the third injection pen in an assembled state during dose setting.

FIG. 69 shows a side view of the third injection pen in a preassembled state.

FIG. 70 shows a detailed view of a longitudinal cut through the distal end of the third injection pen in an assembled state during dose setting.

FIG. 71 shows a detailed view of a further longitudinal cut through the distal end of the third injection pen in the assembled state during dose setting.

FIG. 72 shows a detailed view of a longitudinal cut through the distal end of the third injection pen in the assembled state during dose delivery.

FIG. 73 shows a detailed view of a further longitudinal cut through the distal end of the third injection pen in the assembled state during dose delivery.

FIG. 74 shows a detailed view of a longitudinal cut through the distal end of the third injection pen in the preassembled state.

FIG. 75 shows a detailed view of a further longitudinal cut through the distal end of the third injection pen in the preassembled state.

FIG. 76 shows a perspective view of a fourth injection pen according to the present disclosure.

FIG. 77 shows a side view of the fourth injection pen in an assembled state during dose setting.

FIG. 78 shows a side view of the fourth injection pen in a preassembled state with an adjusting element in a preassembled position.

FIG. 79 shows a side view of the fourth injection pen in the preassembled state with the adjusting element in an adjusting position.

FIG. 80 shows an exploded view of the fourth injection pen.

FIG. 81 shows a longitudinal cut through a dose delivery mechanism of the fourth injection pen in the assembled state during dose setting.

FIG. 82 shows a perspective view of a coupling element of the fourth injection pen

FIG. 83 shows a radial cut through the coupling element shown in FIG. 82 along the line A-A.

FIG. 84 shows a perspective view of a piston rod of the fourth injection pen.

FIG. 85 shows a perspective proximal view of an adjusting element of the fourth injection pen.

FIG. 86 shows a side view of the adjusting element of the fourth injection pen.

FIG. 87 shows a first longitudinal cut through the adjusting element along the line A-A in FIG. 86.

FIG. 88 shows a further side view of the adjusting element of the fourth injection pen in a direction perpendicular to the direction of FIG. 86.

FIG. 89 shows a second longitudinal cut through the adjusting element along the line B-B in FIG. 88.

FIG. 90 shows a perspective distal view of a sleeve of the fourth injection pen.

FIG. 91 shows a perspective distal view of an insert of the sleeve.

FIG. 92 shows a perspective proximal view of the insert of the sleeve.

FIG. 93 shows a perspective distal view of an outer part of the sleeve.

FIG. 94 shows a side view of a coupling member of the fourth injection pen.

FIG. 95 a longitudinal cut through the coupling member along the line A-A in FIG. 90.

FIG. 96 shows a radial cut through the coupling member along the line B-B in FIG. 90.

FIG. 97 shows a perspective distal view of a dosing member of the fourth injection pen.

FIG. 98 shows a perspective view of a longitudinal cut through the dosing member.

FIG. 99 shows a longitudinal cut through the distal end of the dose delivery mechanism of the fourth injection pen in the assembled state during dose setting.

FIG. 100 shows a further longitudinal cut through the distal end of the dose de-livery mechanism of the fourth injection pen in the assembled state during dose setting.

FIG. 101 shows a longitudinal cut through the distal end of the dose delivery mechanism of the fourth injection pen in the assembled state during dose delivery.

FIG. 102 shows a further longitudinal cut through the distal end of the dose de-livery mechanism of the fourth injection pen in the assembled state during dose de-livery.

FIG. 103 shows a longitudinal cut through a distal end of a dose delivery mechanism of the fourth injection pen in a preassembled state with the adjusting element in an preassembled position.

FIG. 104 shows a further longitudinal cut through the distal end of the dose de-livery mechanism of the fourth injection pen in the preassembled state with the adjusting element in the preassembled position.

FIG. 105 shows a longitudinal cut through the distal end of the dose delivery mechanism of the fourth injection pen in the preassembled state with the adjusting element in an adjusting position.

FIG. 106 shows a further longitudinal cut through the distal end of the dose de-livery mechanism of the fourth injection pen in the preassembled state with the adjusting element in the adjusting position;

FIG. 107 a method for at least partly assembling the dose delivery mechanism of the fourth injection pen.

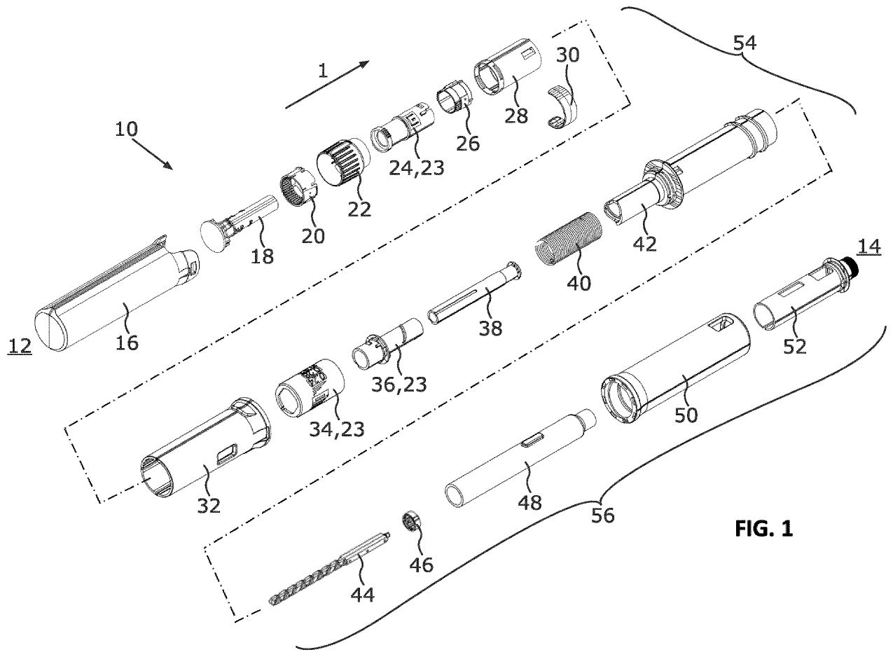

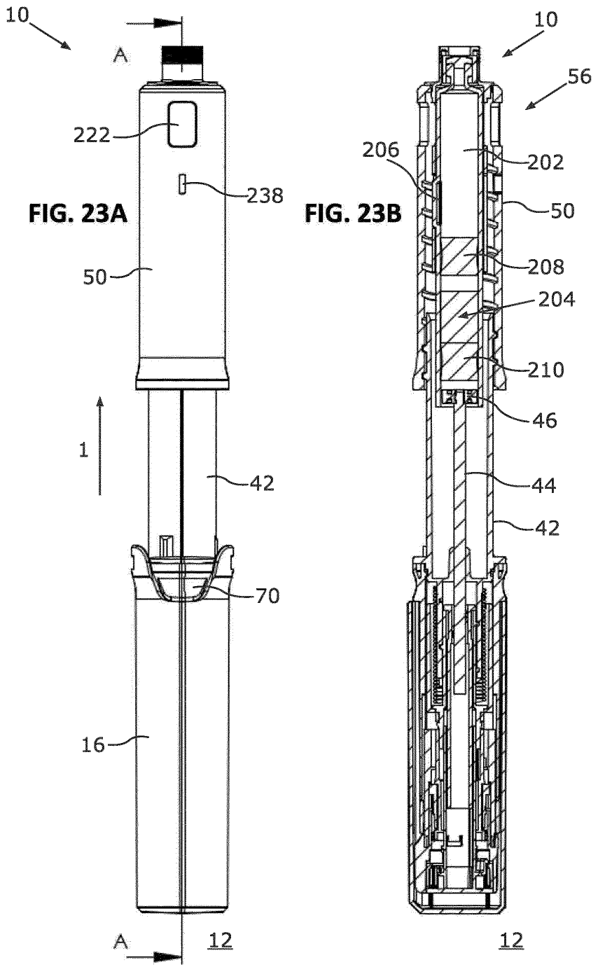

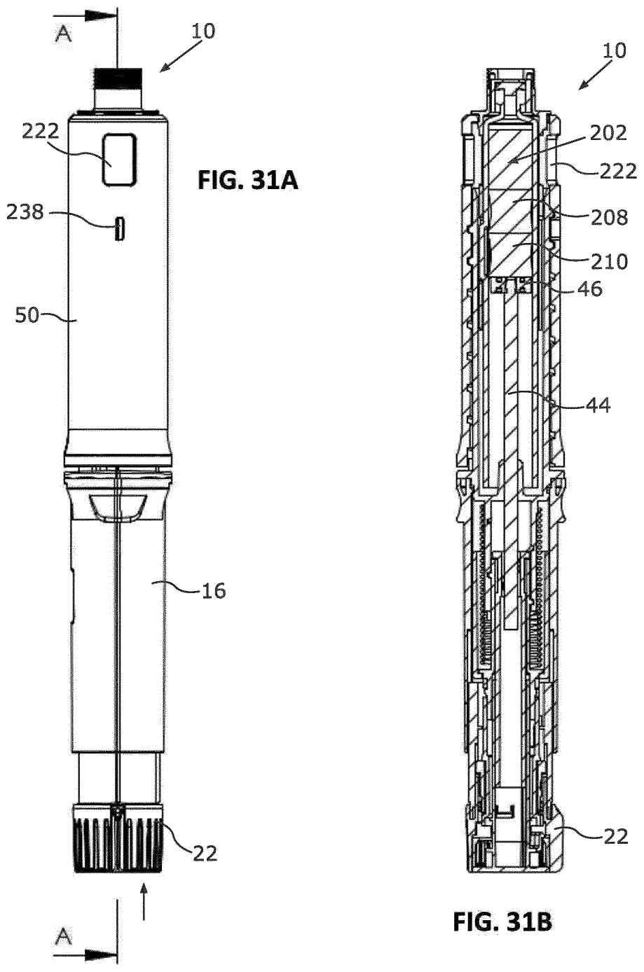

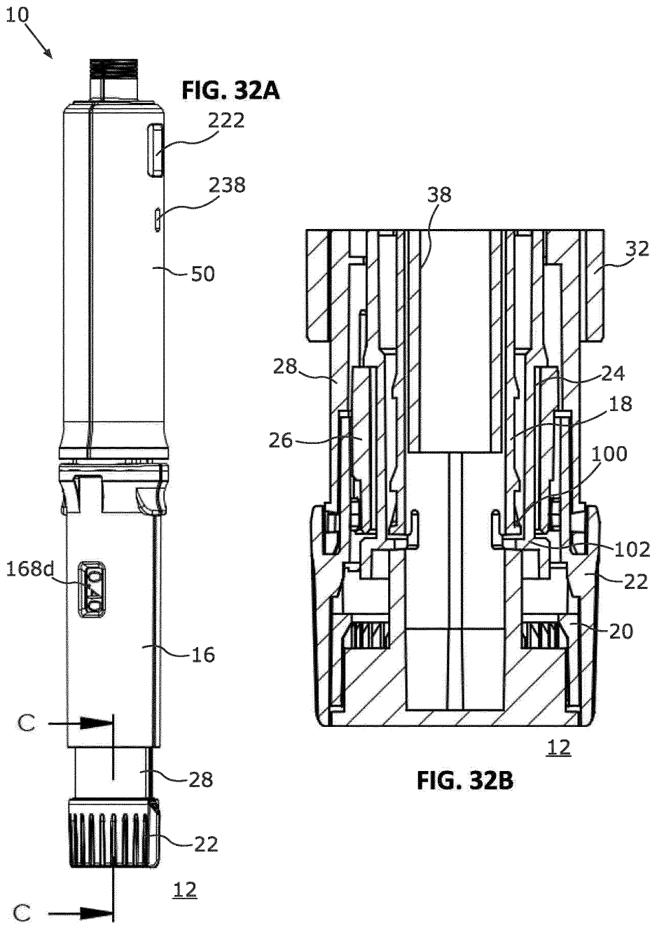

With reference to FIGS. 1 to 22B, parts of an injection pen 10 according to the disclosure are described. Afterwards, with reference to FIGS. 23A to 33B it is described how the pen is meant to be used.

### DETAILED DESCRIPTION

FIG. 1 shows an exploded view of a medicament delivery device in form of an injection pen 10. The injection pen 10 comprises-in an order from a distal end 12 to a proximal end 14-a knob cover 16 that can also be called knob lock, cover or holding element, an injection button 18 that can be part of an actuation member, a snap ring 20, a dose setting knob 22 that can also be called dose setting element, dose adjusting member or knob and can be part of an actuation member, a snap element 24 that can also be called dose setting device, a connector 26, a dose selector 28, a knob key 30 that can also be called a clip, a housing 32 that can also be called body, a dose setting sleeve 34 that can also be called dose sleeve or dose indication member, a driver 36, a nut 38, a spring 40, a piston rod guide 42 that can also be called piston guide, a piston rod 44, a piston disc 46 that can also be called a bearing, a dual chamber cartridge 48 that can also be called a medicament container, a fluid compartment or a cartridge, a cartridge container 50, and a cartridge holder or cartridge key 52. The assembly of the cartridge container 50 and the cartridge holder 52 can also be called medicament container holder. Thereby, the cartridge container 50 provides an outer container holder and the cartridge key 52 provides an inner container holder of the container holder.

While the above mentioned parts of the injection pen 10 can each be formed as separate parts to simplify production of the separate parts, it would be generally possible to form one or more of the parts integrally with each other. For example, the injection button 18, the snap ring 20, and/or the dose setting knob 22 could be formed integrally with each other. Furthermore, the dose setting sleeve 34 and the driver 36 could be formed integrally with each other. Generally, even the housing 32 and the piston rod guide 42 could be formed integrally with each other.

The different parts can be grouped together to define different functional units.

E.g. the section between the injection button 18 and the piston rod guide 42 can be called a dose setting mechanism 54, a dose setting unit, a dose delivery mechanism and/or a dose de-livery activation mechanism. On the other hand, the section between the piston rod guide 42 and the cartridge key 52 can be called drug reconstitution unit 56 or reconstitution means or element. The cartridge container 50 and the cartridge holder 52 can be called a cartridge holding unit. The cartridge container 50, the cartridge holder 52, and the cartridge 48 can be called a cartridge unit. For example, the cartridge unit can be sold—in a preassembled state or as separate parts—separately from the rest of the injection pen 10.

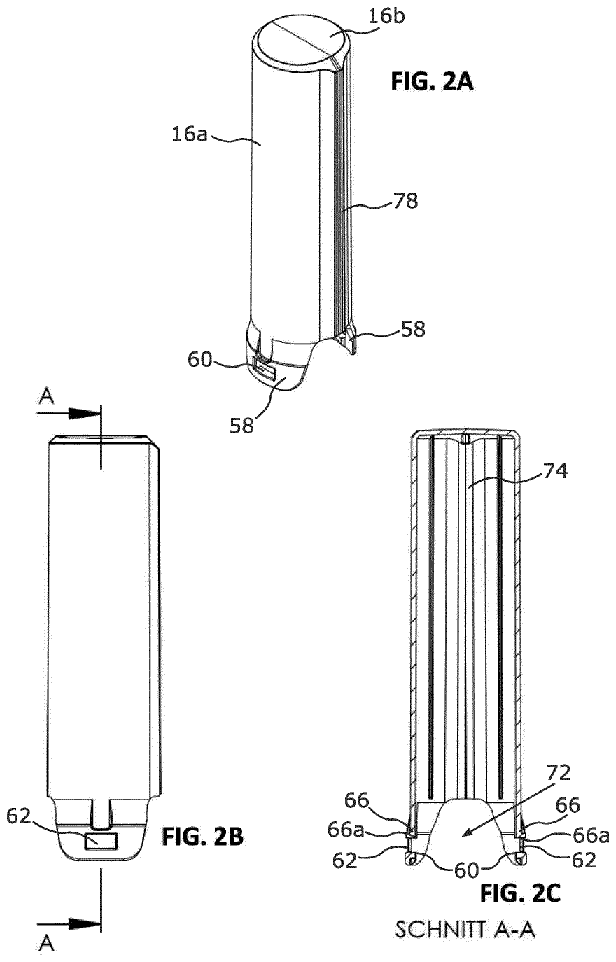

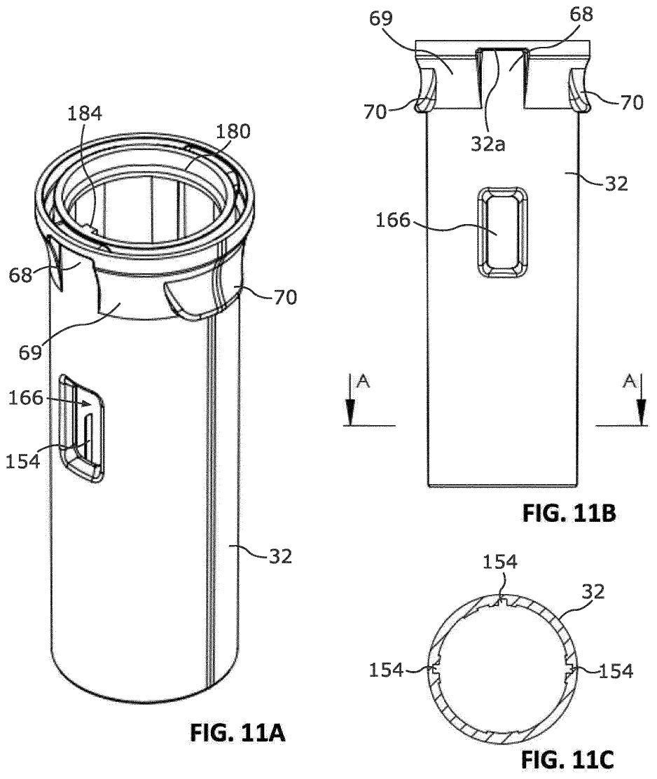

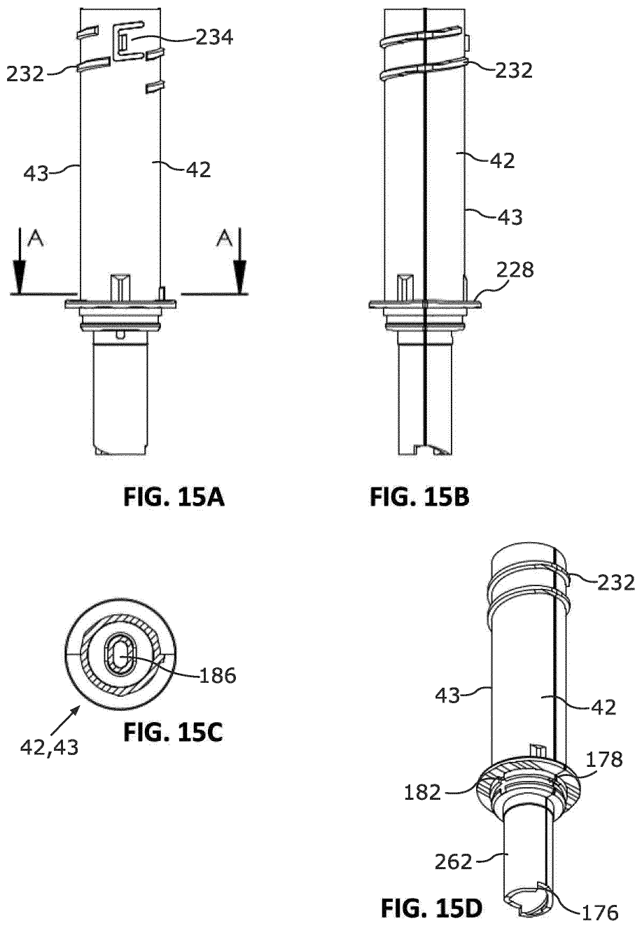

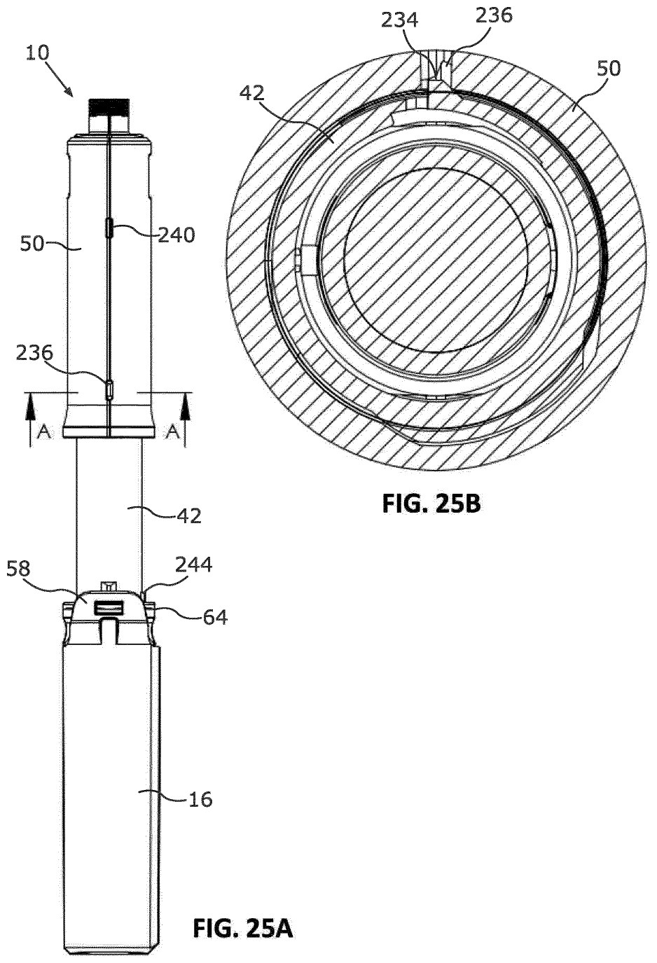

Next, the above-mentioned parts of the injection pen 10 are described in the order starting from the distal end 12 and ending at the proximal end 14, the distal end 12 and the proximal end 14 being opposite ends of the injection pen 10 and the proximal end 14 comprising a dispensing outlet:
FIGS. 2A to 2C depict the knob cover 16. The knob cover 16 covers the dose setting knob 22 during delivery, i.e. shipping, of the injection pen 10 to a costumer, e.g. the patient. The knob cover 16 is fully detachable from the rest of the injection pen 10. The knob cover 16 is attachable to the housing 32 and/or detachable from the housing 32 via two deformable wings 58 that can be deflected outwardly, i.e. in a radial direction, to detach the knob cover 16 from the housing 32. The wings 58 form a proximal end section of the knob cover 16. On an inner surface of each of the wings 58, form-fitting engagement means in the form of a lug 60 are provided, that are configured to engage with the housing 32, in particular with a radially extending coupling surface 228 (cf. FIG. 15C) formed on the piston guide 42, to axially fixate the knob cover 16 relative to the housing 32 in a distal direction. Next to each of the lugs 60, one window 62, i.e. a radially extending opening, is formed in the wings 58. When the knob cover 16 is attached to the housing 32, the windows 62 are positioned at an axial position where the housing 32 forms a circumferentially extending elevation 64 (cf. FIG. 25A). On the distal side of each window 62, i.e. away from the lugs 60, on the inner side surface of the respective wing 58, an abutment 66 is formed. The abutment 66 has a width that is adapted to a width of a recess or cut-out 68 (cf. FIG. 11A) on an outer surface of the housing 32, more precisely in a chamfered portion 69 formed on the outer surface of the housing 32. Furthermore, the abutment 66 forms a front surface 66a that axially abuts a radially extending surface 32a (cf. FIG. 11B) defining a proximal end of the cut-out 68 when the knob cover 16 is attached to the housing 32. The radially extending surface 32a defines a stop surface that stops proximal movement of the knob cover 16 relatively to the housing 32, e.g. if the injection pen 10 is dropped onto a floor with the distal end 12 first. In order to further ensure that the knob cover 16 does not move past its attached position in the proximal direction 1, axial abutment elevations 70 (cf. FIG. 11A) can be formed on the outer surface of the housing 32. The elevations 70 are configured to engage with clearances 72 (cf. FIG. 2C) formed between the wings 58 so that proximal front surfaces of the knob cover 16 abut distal front faces of the axial abutment elevations 70.

A form-fitting engagement between the abutments 66 and the cut-outs 68 and/or a form-fitting engagement between the elevations 70 and the clearances 72 make sure that the knob cover 16 is rotationally constrained relative to the housing 32 when the knob cover 16 is attached to the housing 32.

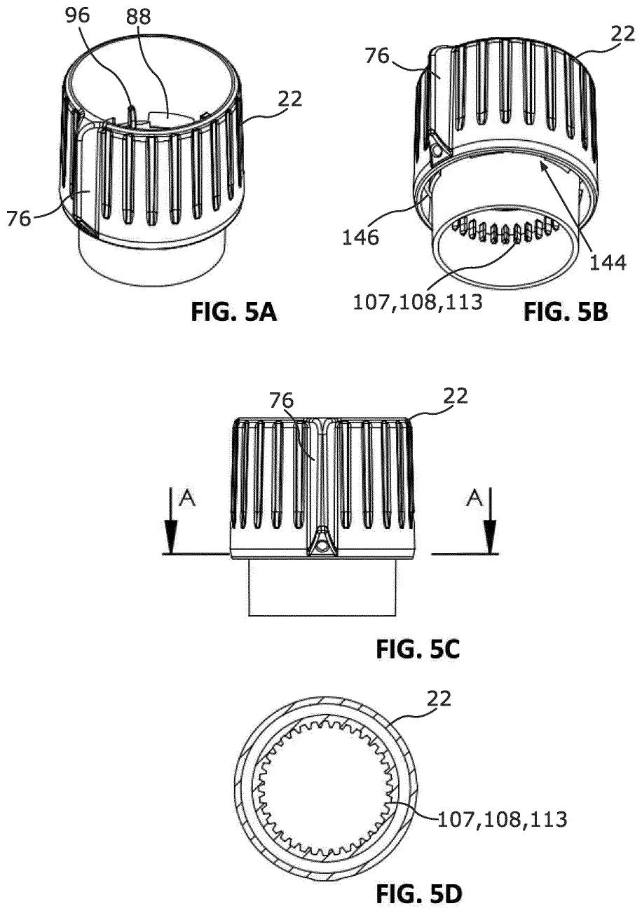

As can be seen from FIG. 2A, the knob cover 16 is only detachable from the rest of the injection pen 10 by moving the knob cover 16 linearly in a distal direction. In order to do so, a linear recess 74 is formed on the inner circumferential surface of the knob cover 16 that corresponds to an anti-rolling means or element 76 (cf. FIG. 5B) of the dose setting knob 22 in the form of an axially extending rib. Therefore, the dose setting knob 22 is blocked from rotating inside the knob cover 16 by the form-fitting engagement of the linear recess 74 and the anti-rolling means 76. The knob cover 16, as can be seen in FIG. 2A, also forms anti-rolling means 78 in form of an axially extending rib on the outer surface of the knob cover 16. The anti-rolling means 76 and 78 make sure that the injection pen 10 and the knob cover 16 do not roll away when placed on a flat surface. As can be also seen from FIG. 2A, the knob cover 16 has a closed circumference 16a and a closed face 16b at its distal end. Therefore, the knob cover 16 forms a closed sleeve around the distal section of the injection pen 10.

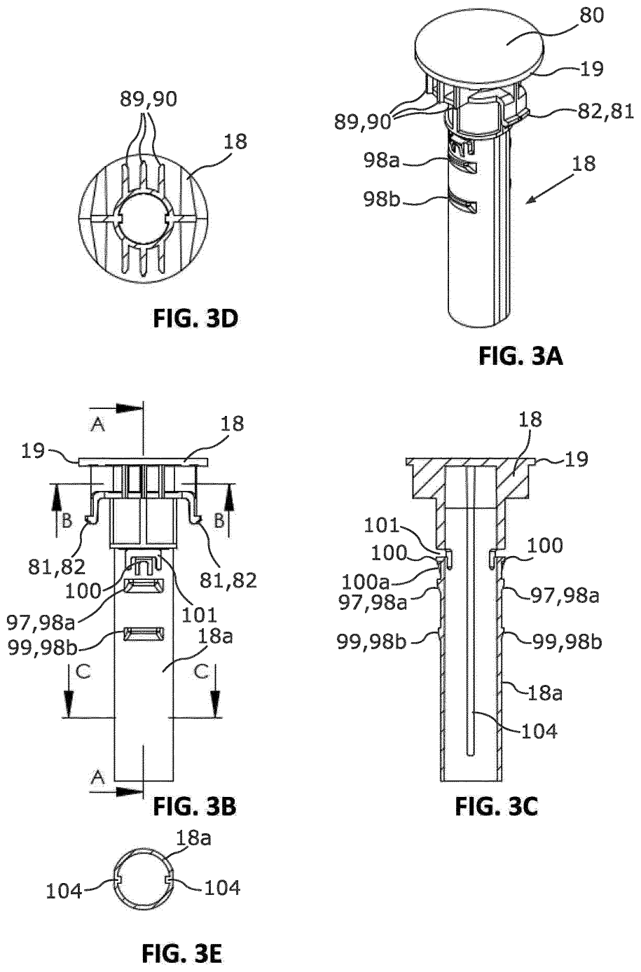

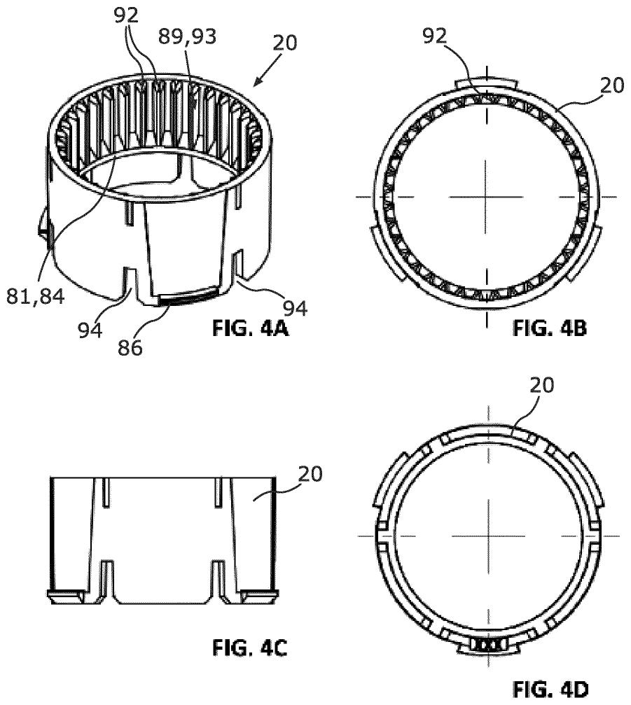

FIGS. 3A to 3E depict the injection button 18. The injection button 18 forms a distal front surface 80 to apply a force to the injection button 18 to inject a set dose. The injection button 18 comprises axial fixation means or element 82 to axially attach the injection button 18 to the snap ring 20 (cf. FIG. 4A-4D) which is axially connected to the dose setting knob 22 (cf. FIG. 5A to 5D). The axial fixation means 82 comprise two elastically deformable hooks 82 which engage with a circumferentially extending rib 84 on the snap ring 20. The snap ring 20 also comprises axial fixation means or element 86 in the form of elastically deformable bendable hooks that engage with an undercut 88 formed in the dose setting knob 22. The injection button 18, the snap ring 20 and the dose setting knob 22 are permanently axially fixed to each other in an assembled state of the dose delivery mechanism 54.

The injection button 18 also forms rotation fixation means or element 90 in the form of radially extending ribs. The ribs 90 are form-fittingly engaged with rotation fixation means or element 92 (cf. FIG. 4A) in the form of teeth arranged in an inner circumferential surface of the snap ring 20 to rotationally connect the injection button 18 to the snap ring 20. The rotation fixation means 92 form a toothed part 93 of the snap ring 20 and the ribs 90 form an engaging part of the injection button 18. The snap ring 20 comprises rotation fixation means or element 94 in the form of axially extending recesses that define side surfaces of the elastically deformable bendable hooks 86 and that engage with rotation fixation means 96 or element in the form of axially extending ribs (cf. FIG. 5A) on the inner circumferential surface of the dose setting knob 22.

After assembly and in an assembled state of the dose delivery mechanism 54, the injection button 18, the snap ring 20 and the dose setting knob 22 are rigidly connected with each other and form both a dose setting member and an actuation member of the dose delivery mechanism 54.

The injection button 18 forms a cylindrical portion 18a. On the cylindrical portion 18a, assembling means or element 98 in the form of elevations are formed to axially preassemble the injection button 18 with the snap element 24. More precisely, the lower, i.e. proximal, assembling means 98b or element (cf. FIG. 3C) restricts distal movement of the injection button 18 relative to the snap element 24 by interfering with coupling means or element 102 on the snap element 24. The upper, i.e. distal, assembling means 98a or element restricts proximal movement of the injection button 18 relative to the snap element 24 by interfering with coupling means 102 on the snap element 24 after pre-assembly and distal movement of the injection button 18 after final assembly. When the snap element 24 and the injection button 18 are preassembled, i.e. in a preassembled state, the coupling means 102 is arranged between the proximal assembling means 98b and the distal assembling means 98a. In said preassembled state, the injection button 18 is not yet rigidly connected to the snap ring 20 and the dose setting knob 22. However, when the coupling means 102 is arranged distally from the distal assembling means 98a, i.e. in the assembled state, the injection button 18 is rigidly connected to the snap ring 20 and the dose setting knob 22. The injection button 18 also forms coupling means 100 in the form of protrusions being arranged on an outer circumferential surface of the injection button 18 on elastically inwardly bendable portions. The inwardly bendable portions extend in an axial direction and are sectionally surrounded by cut-outs 101.

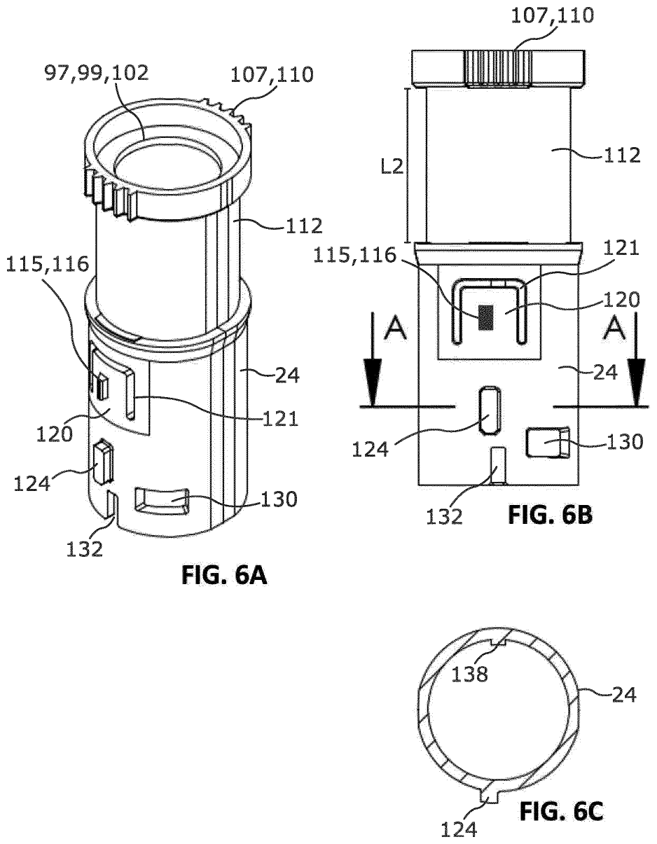

The coupling means 100 are configured to permanently axially lock the injection button 18 and therefore also the snap ring 20 and the dose setting knob 22 to the snap element 24 after the injection has been completed to render the injection pen 10 inoperable. Namely, when the injection button 18 is moved axially to initiate the dose delivery, the coupling means 100 pass the radially inwardly extending coupling means in the form of a circumferentially extending ledge 102 (cf. FIG. 6A) on the snap element 24. The radially inwardly extending ledge 102 causes the protrusions being arranged on elastically inwardly bendable portions 100 to bend inwardly until the protrusions have passed the ledge 102. In order to reduce the force needed to push the protrusions 100 past the ledge 102, the protrusions 100 form chamfered outer surfaces 100a. Alternatively or additionally, the ledge 102 could form a chamfered inner surface. When the protrusions 100 have passed the ledge 102, they snap back into their neutral position which causes the injection button 18 to be permanently axially locked relative to the snap element 24. This feature makes sure that the injection pen 10 can only be used one single time to inject exactly one dose.

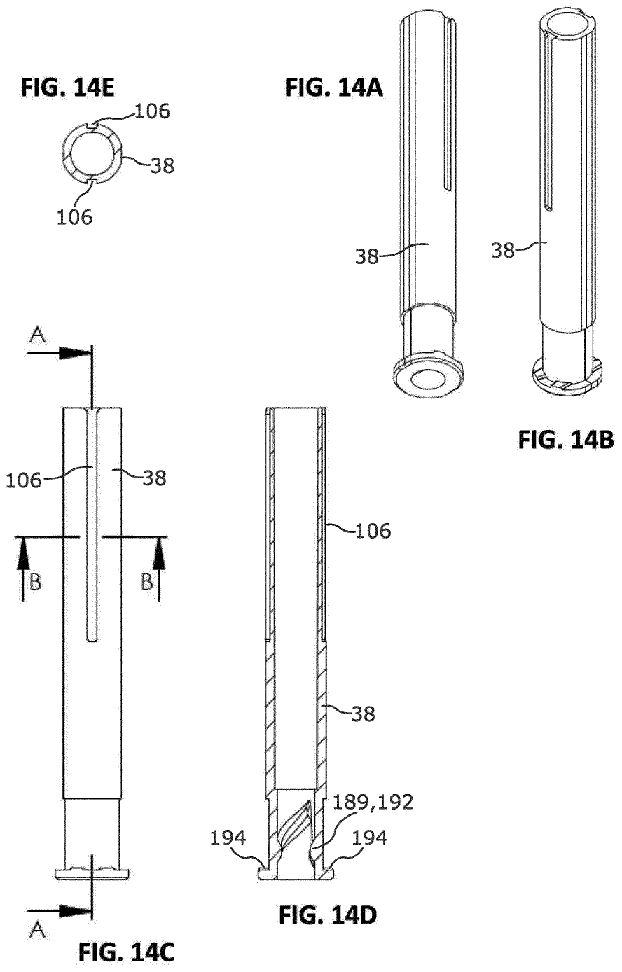

As can be best seen in FIGS. 3C and 3E, the injection button 18 comprises an axially extending rib 104 on its inner circumferential surface. The axially extending rib 104 engages in an axially extending groove 106 of the nut 38 (cf. FIG. 14C) to form rotation fixation means. Due to the axially extending rib 104 and the corresponding axially extending groove 106, the injection button 18 and the nut 38 can move axially relative to each other but are rotationally fixed to each other.

As can be best seen on FIGS. 5B and 5D, a set of teeth 108 are formed in an axial section on an inner circumferential side of the dose setting knob 22. These teeth 108 are configured to mesh with a set of teeth 110 arranged in a distal section on an outer circumferential surface of the snap element 24 (cf. FIG. 6A-6C) during dose setting. Therefore, when the dose setting knob 22 is rotated during dose setting, the snap element 24 is rotated too. As can be seen in FIG. 6A, the teeth 110 are arranged only in two opposite sections of the snap element 24 while sections between the two opposite sections do not form any teeth. This has ad-vantages regarding production of the injection pen 10. Alternatively, the teeth 110 could be formed along the whole circumference of the snap element 24.

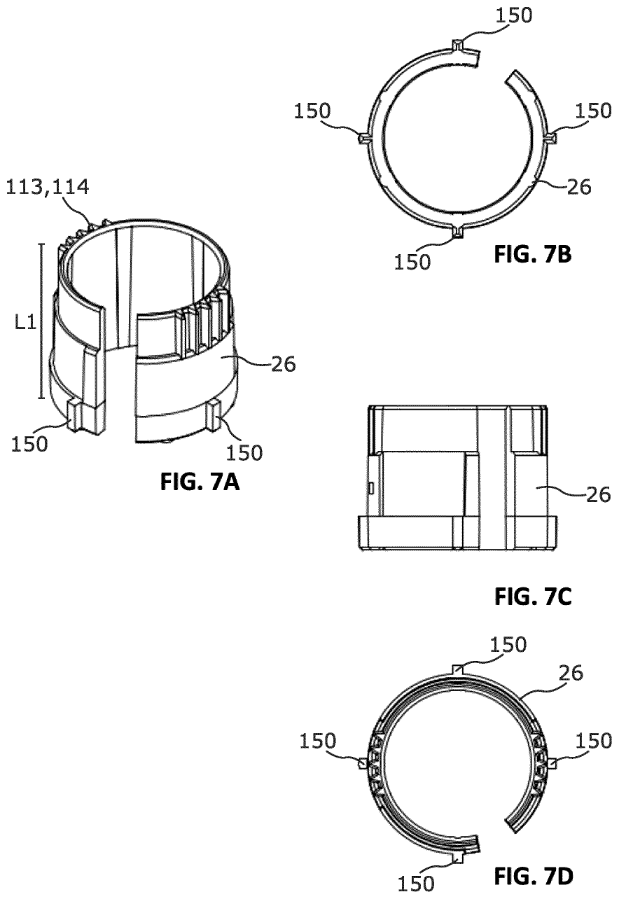

The snap element 24 forms an axial section with a reduced cross section forming a coupling surface 112 for the connector 26. The connector 26 shown in the figures is formed as an integral part. However, the connector 26 could also be formed from multiple, e.g. two, parts connected to each other, e.g. via a form-fitting connection. The connector 26 has an open cross section (cf. FIG. 7D) so it is clippable onto the snap element 24 at the reduced cross section. The connector 26 is axially fixedly connected to the snap element 24 in both directions due to the connector 26 having a length L1 in the axial direction that corresponds to a length L2 in the axial direction of the axial section with the reduced cross section. However, the connector 26 is rotatable relative to the snap element 24. When the dose setting knob 22 is pushed in the proximal direction 1 to initiate dose delivery, the teeth 108 of the dose setting knob 22 engage with a set of teeth 114 formed on an outer circumferential surface of the connector 26 instead of the teeth 110 of the snap element 24 so that the snap element 24 can rotate relative to the dose setting knob 22 during dose delivery. The engagement between the teeth 108 of the dose setting knob 22 and the teeth 114 of the connector 26 makes sure that the dose setting knob 22 does not rotate during dose delivery with respect to the housing 32 due to connector 26 being rotationally fixed to the housing 32 via the dose selector 28.

The snap element 24 further comprises an engagement feature 116 in the form of an axially extending radial projection. The engagement feature 116 is an axially extending rib. The engagement feature 116 can have a symmetrical cross section in a radial plane perpendicular to a longitudinal axis of the injection pen 10 or an asymmetrical cross section.

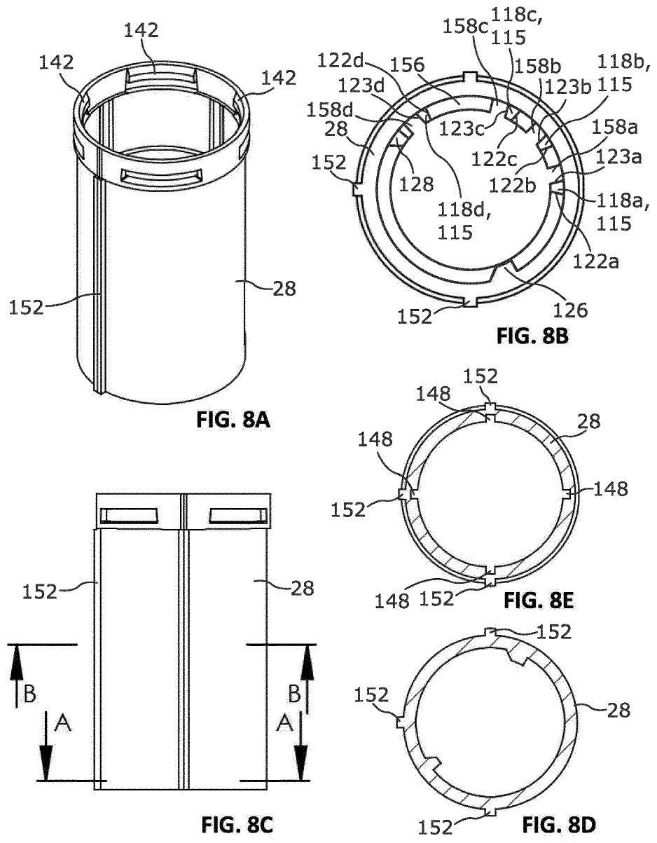

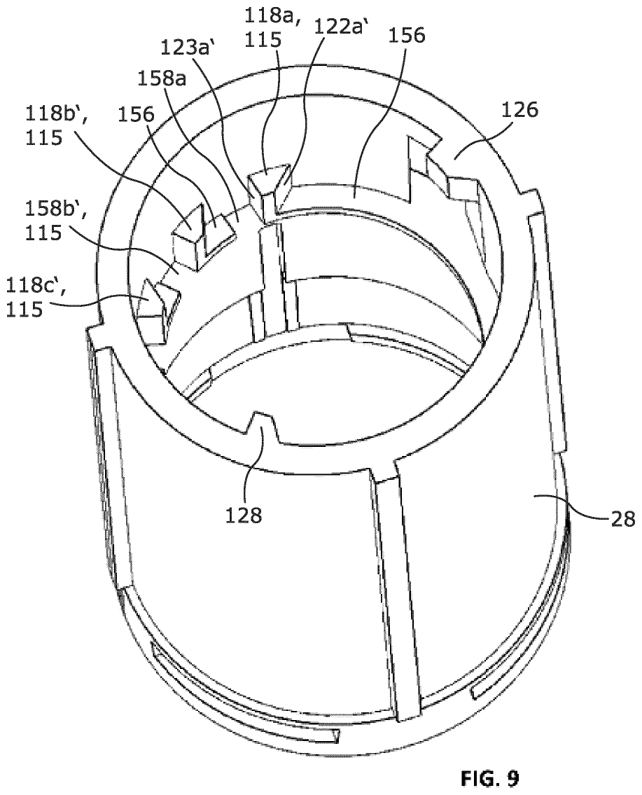

The engagement feature 116 is configured to engage with dose stops 118a, 118b, 118c, and 118d (cf. FIG. 8B) formed on an inner circumferential surface of the dose selector 28 to set a desired dose. Therefore, the engagement feature 116 is used as a dose definition element and the engagement feature 116 together with the dose stops 118a, 118b, 118c, 118d form a dose definition mechanism 115 of the injection pen 10. The dose definition element 116 is located on an elastically deformable section 120, i.e. an axially extending arm partially surrounded by a cut-out 121. The elastically deformable section 120 bends inwardly when the dose definition element 116 passes one of the dose stops 118a, 118b, 118c, and 118d. In order to reduce the force needed to rotate the dose setting knob 22 and the snap element 24 relative to the dose selector 28 to enlarge or decrease the set dose, the dose stops 118 have chamfered side surfaces 122a-d and 123a-d. According to the embodiment shown in FIG. 8B, the dose stops 118a-d have a symmetrical cross section in the radial plane perpendicular to the longitudinal axis of the injection pen 10. In other words, the chamfered side surfaces 122 and 123 have pitches that are equal to each other regarding their amount. According to another embodiment shown in FIG. 9, chamfered side surfaces 122a′-d′ that get in contact with the projection 116 to deform the elastically deformable section 120 when the dose is set to a higher dose have a smaller pitch than chamfered side surfaces 123a′-d′ that get in contact with the projection 116 when the dose is set to a lower dose. The side surfaces 123a-d define rotational positions corresponding to settable doses. The spring 40 is configured to rotate the snap element 24 relative to the dose selector 28 so that the dose definition element 116 abuts one of the side surfaces 123a-d.

The snap element 24 further comprises a hard stop 124 in the form of an axially extending rib that abuts a hard stop 126 formed on the dose selector 28 when the injection pen 10 is delivered to a costumer. The hard stop 126, contrary to known pens, does not correspond to a zero-dose stop but instead corresponds to a pre-set dose stop. A further discussion regarding this feature follows. The hard stop 124 is axially distanced from the dose definition element 116 but axially aligned with the dose definition element 116. The hard stop 124 is configured to abut an end of dose setting hard stop 128.

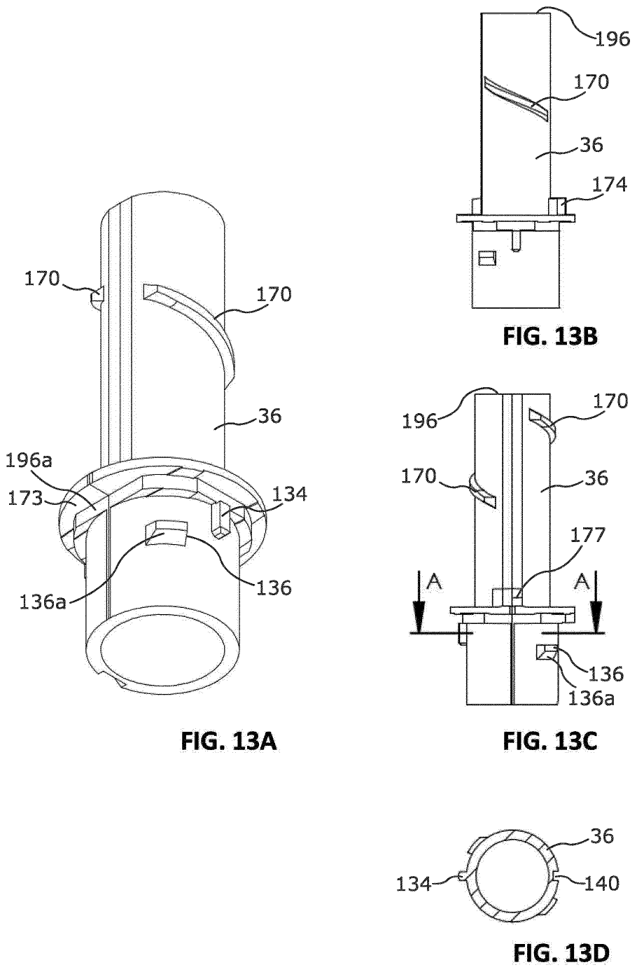

The snap element 24 further comprises axial and rotational fixation means in the form of a radially extending opening 130 and an axially extending slot 132 to axially and rotationally fix the snap element 24 to the driver 36. As can be seen in FIG. 13A, the driver 36 has an axially extending rib 134 that is configured to engage with the slot 132 of the snap element 24. Furthermore, the driver 36 has a protrusion 136 with a chamfered surface 136a that engages with the opening 130 of the snap element 24. While the opening 130 and the protrusion 136 form the axial fixation means, the slot 132 and the rib 134 form the rotational fixation means. Due to the axial and rotational fixation means, the snap element 24 and the driver 36 can be connected to each other in one defined relative rotational position. In order to strengthen the rotational fixation between the snap element 24 and the driver 36, an axially extending rib 138 is formed on an inner circumferential surface of the snap element 24 (cf. FIG. 6C) that engages with an axially extending groove 140 (cf. FIG. 13D) on an outer circumferential surface of the driver 36.

FIGS. 8A to 8E depict the dose selector 28. The dose selector 28 comprises axial fixation means or element 142 in the form of circumferentially extending projections on an inner circumferential surface of a distal section of the dose selector 28. The dose selector 28 is axially fixed to the dose setting knob 22 by inserting the distal section with the axial fixation means 142 into a circumferentially extending intake 144 (cf. FIG. 5B). In the intake 144, the dose setting knob 22 forms axial fixation means or element 146 in the form of circumferentially extending protrusions on an outer circumferential surface which get engaged with the axial fixation means 142 of the dose selector 28 to form an axial connection that allows relative rotational movement between the dose selector 28 and the dose setting knob 22.

As can be seen best on FIG. 8E, rotation fixation means or element 148 in the form of axially extending grooves are formed on an inner circumferential surface of the dose selector 28. The rotation fixation means 148 are engaged with rotation fixation means or element 150 in the form of axially extending ribs formed on the outer circumferential surface of the connector 26 (cf. FIG. 7B). The rotation fixation means 148, 150 allow axial movement between the dose selector 28 and the connector 26. The dose selector 28 further comprises rotation fixation means or element 152 in the form of axially extending ribs formed on an outer circumferential surface of the dose selector 26. The rotation fixation means 152 engage with rotation fixation means or element 154 in the form of axially extending grooves formed on the inner circumferential surface of the housing 32 (cf. FIG. 11C). The rotation fixation means 152, 154 are configured to define one single possible rotational alignment that allows insertion of the dose selector 28 into the housing 32. The rotation fixation means 150, 152 allow axial movement between dose selector 28 and the housing 32.

In order to define deliverable doses, the dose selector 28 (cf. FIG. 8B) forms a circumferentially extending rib 156 with cut-outs 158a, 158b, 158c, and 158d. The cut-outs 158a, 158b, 158c, and 158d are assigned to the respective dose stops 118a, 118b, 118c, and 118d. The rib 156 with its cut-outs 158a, 158b, 158c, and 158d makes sure, that injection is only possible if the dose definition element 116 of the snap element 24 is at an angular position relating to one of the cut-outs 158a, 158b, 158c, and 158d, i.e. relating to one of the settable doses. If the dose definition element 116 is not at an angular position relating to one of the cut-outs 158a-d, axial movement of the dose definition element 116, and therefore the snap element 24, relative to the dose selector 28 is blocked by the circumferentially extending rib 156. As can be seen from FIG. 8B, there is no cut-out assigned to the pre-set dose hard stop 126. Therefore, starting an injection is inhibited when the injection pen 10 is set to the pre-set dose.

According to an alternative embodiment, the circumferentially extending rib 156 could be arranged to interact with the hard stop 124 instead of the dose definition element 116. Therefore, injection would only be possible if the hard stop 124 of the snap element 24 would be at an angular position relating to one of the cut-outs 158a, 158b, 158c, and 158d, i.e. relating to one of the settable doses.

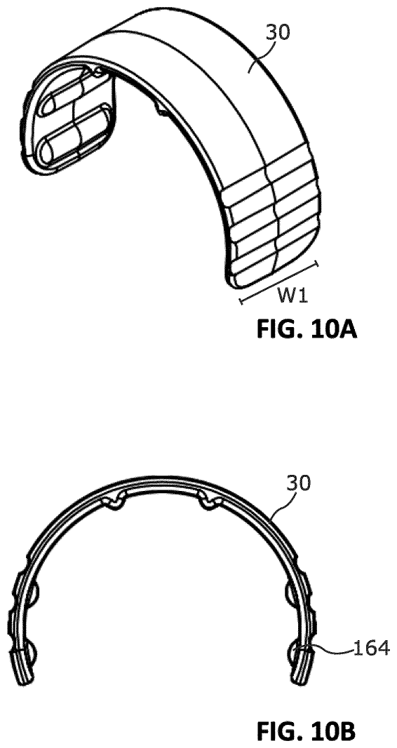

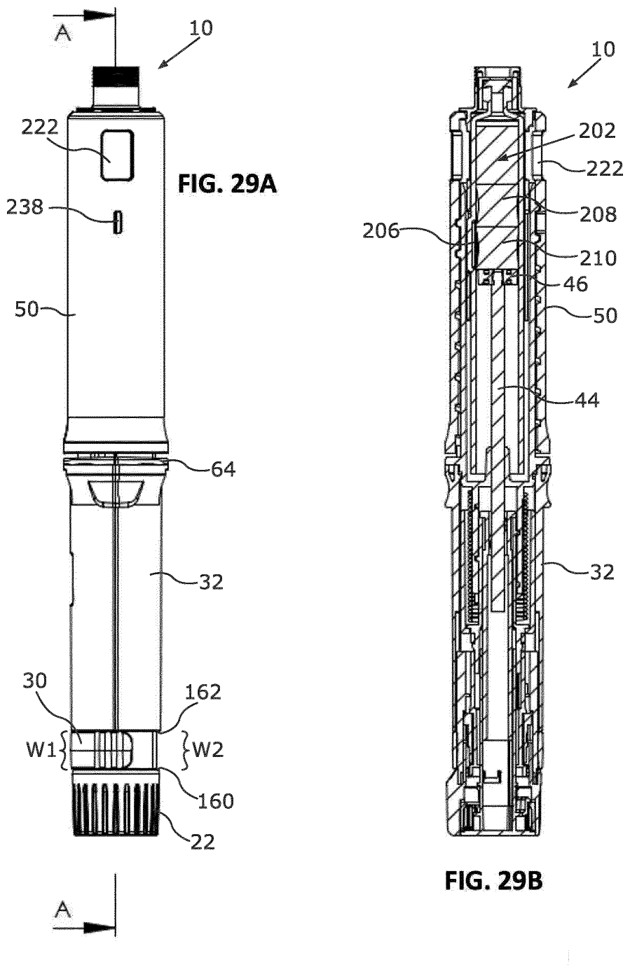

FIGS. 10A and 10B depict the knob key 30. The knob key 30 is configured to be attached to the outer circumferential surface of the dose selector 28 to keep the dose setting knob 24 from unintentionally moving in the proximal direction 1 relative to the housing 32 if the injection pen 10 in an as-delivered state drops onto its proximal end. The clip element 30 has a width W1 that corresponds to a width W2 (cf. FIG. 29A) between a proximal edge 160 of the dose setting knob 22 and a distal edge 162 of the housing 32. The knob key 30 is C-shaped and has holding protrusions 164 that interact with the rotation fixation means 152 on the outer circumferential surface of the dose selector 28 to attach the knob key 30 to the dose selector 28. The knob key 30 can be taken off the dose selector 28 by slightly bending the C-shaped knob key 30. In the as-delivered state, the knob cover 16 extends around the knob key 30 to hold the knob key 30 in place. The knob key 30 can only be taken off the dose selector 28 after the knob cover 16 has be removed.

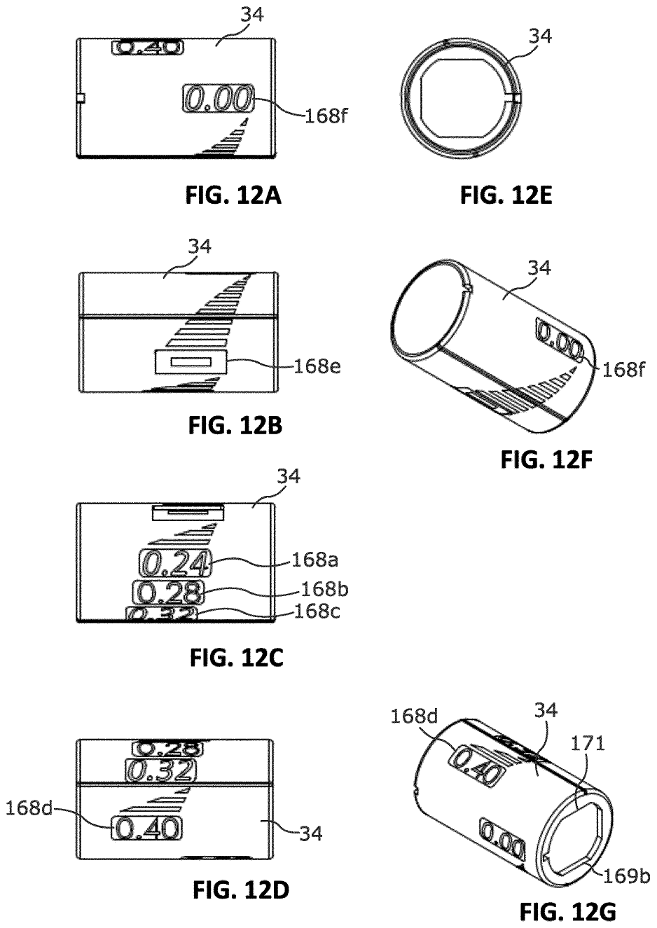

The housing 32 is shown in FIGS. 11A to 11C. The housing 32 forms a viewing window 166 for displaying a state of the injection pen 10, in particular a set dose, indicated by the dose sleeve 34 through the window 166. The dose sleeve 34 rotates relative to the housing during dose setting and dose delivery which causes a change of what is displayed through the window 166. In different circumferential positions along the outer circumferential surface of the dose sleeve 34, labels 168a-168d (cf. FIGS. 12C and 12D) for different settable doses are located. Furthermore, a preset-dose label 168e (cf. FIG. 12B) is located on the dose sleeve 34 that corresponds to a pre-set dose, i.e. an amount of medicament that would be injected if the injection could be started from the pre-set dose. As can be seen from comparing FIGS. 12A and 12B, the pre-set dose label 168e differs from a zero-dose label 168f, i.e. the label that shows that no medicament would be injected if the injection would be started in that state. This zero-dose label 168f is shown through the window 166 when the injection has been completed. The labels 168a, 168b, 168c, and 168d correspond to the settable doses defined by the dose stops 118a, 118b, 118c, and 118d.

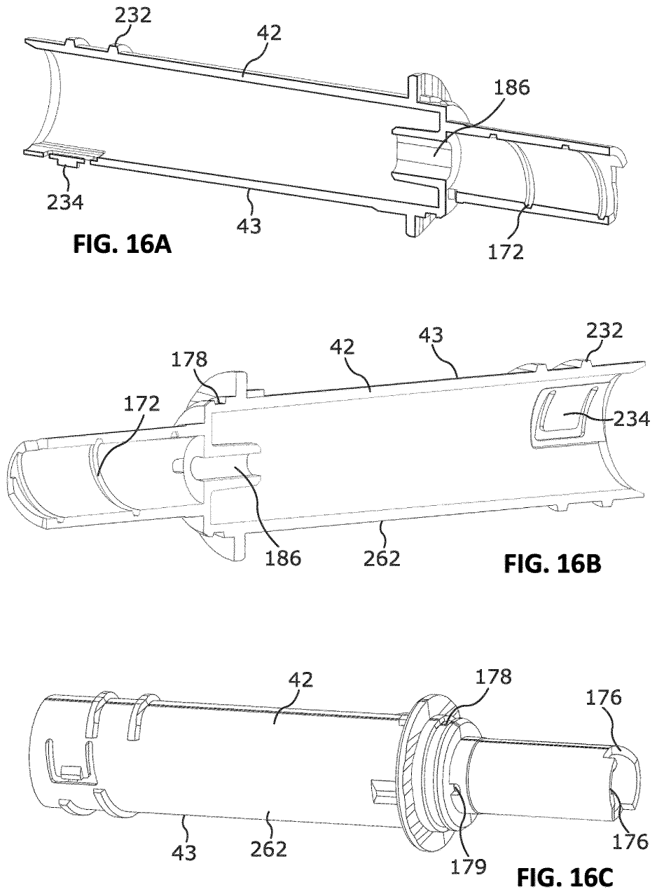

The dose sleeve 34 is rotationally and axially rigidly coupled to the driver 36 (cf. FIG. 13A-13D). In order to rotationally couple the dose sleeve 34 to the driver 36 corresponding out-of-round outer and inner circumferential surfaces 169a and 169b are formed on the driver 36 and the dose sleeve 34, respectively. Furthermore, the dose sleeve 34 forms a fixing section 171 that is pinched between a proximal end of the snap element 24 and a face surface 173 (cf. FIG. 13A) of the driver 36 to axially fix the dose sleeve 34 to the driver 36 and the snap element 24. The driver 36 forms an outer thread 170 that engages with an inner thread 172 (cf. FIGS. 16A-16B) of the piston guide 42. The threaded connection 170, 172 causes the driver 36 to rotate when the driver 36 is moved axially relative to the piston guide 42 and causes the driver 36 to move axially relative to the piston guide 42 when the driver 36 is rotated relative to the piston guide 42. Furthermore, the driver 36 defines end stops 174 that abut end stops 176 of the piston guide 42 at the end of the dose delivery. The surfaces defining the end stops 174, 176 are arranged in parallel to a middle axis of the injection pen and face in a radial direction. The driver 36 also forms attachment means or element 177 in the form of a radially extending hook for attaching one end section of the spring 40 to the driver 36. The other end section of the spring 40 is attached to attachment means or element 179 (cf. FIG. 16C) at the outer circumferential surface of the piston rod guide 42.

According to an alternative version, the dose sleeve and the driver can be formed as separate parts that are axially movable relative to each other but rotationally fixed to each other and both, the dose sleeve and the driver, can have a thread that is threadedly coupled to the housing. The thread of the dose sleeve and the thread of the driver can have different pitches.

The piston guide 42 is axially and radially fixed to the housing 32 and can therefore be considered part of the housing. In order to axially fix the piston guide 42 to the housing 32, axial fixation means or element 178 in the form of a circumferentially extending groove are formed on the piston guide 42 that engage with axial fixation means or element 180 (cf. FIG. 11A) in the form of a circumferentially extending rib formed on an inner circumferential surface of the housing 32. In order to rotationally fix the piston guide 42 to the housing 32, rotation fixation means or element 182 in the form of an axially extending groove are formed on an outer circumferential surface 262 of the piston guide 42 that engage with rotation fixation means or element 184 (cf. FIG. 11A) in the form of an axially extending rib formed on an inner circumferential surface of the housing 32. The axial and rotational fixation means 178, 180, 182, and 184 allow attachment of the piston rod guide 42 to the housing 32 in exactly one relative rotational position.

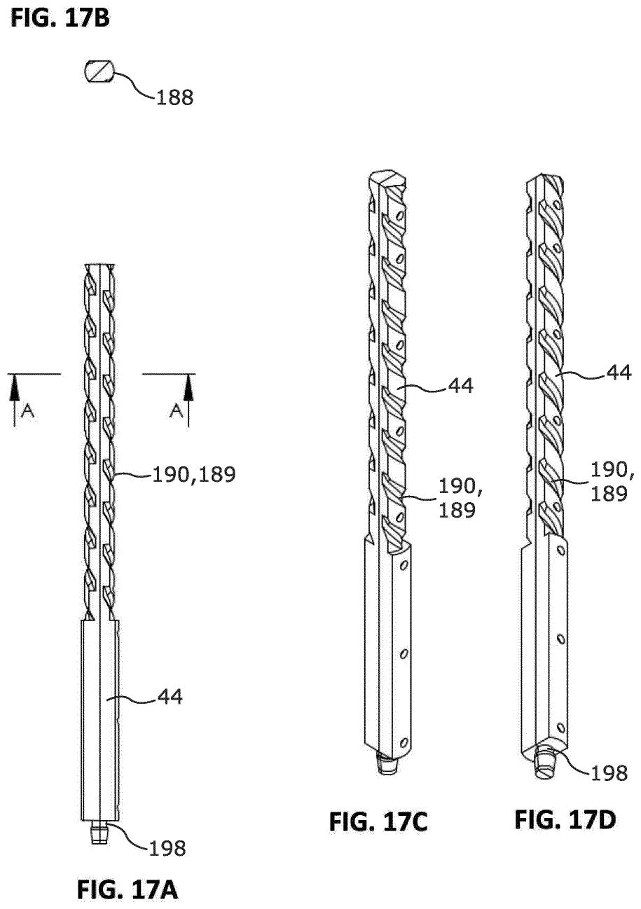

The piston guide 42 has an out of round axial opening 186 (cf. FIG. 15C) that corresponds to an out of round cross-section 188 (cf. FIG. 17B) of the piston rod 44. Therefore, the piston rod 44 is axially movable relative to the piston rod guide 42, but cannot rotate relative to the piston rod guide 42. The piston rod 44 forms an outer thread 190 that is in engagement with an inner thread 192 (cf. FIG. 14D) of the nut 38. The outer thread 190 and the inner thread 192 form a threaded connection 189 between the piston rod 44 as a first threaded element and the nut 38 at a second threaded element. The piston rod 44 and the nut 38 can move relative to each other in a compulsory guided combined axial and rotational movement. In a proximal end section of the nut 38, an annular pressing surface 194 extending in the distal direction is formed on the nut 38. This pressing surface 194 abuts a front surface 196 of the driver 36 during dose delivery. During dose delivery, the driver 36 moves in a combined axial and rotational movement relative to the piston rod guide 42 while the nut 38 is rotationally fixed to the housing 32. In order to reduce friction during dose delivery, a ball bearing and/or a glide disc made of low-friction material can be arranged between the pressing surface 194 and the front surface 196 of the driver 36. In both cases, during dose delivery, the driver 36 pushes the piston rod 44 via the nut 38 in the proximal direction 1.

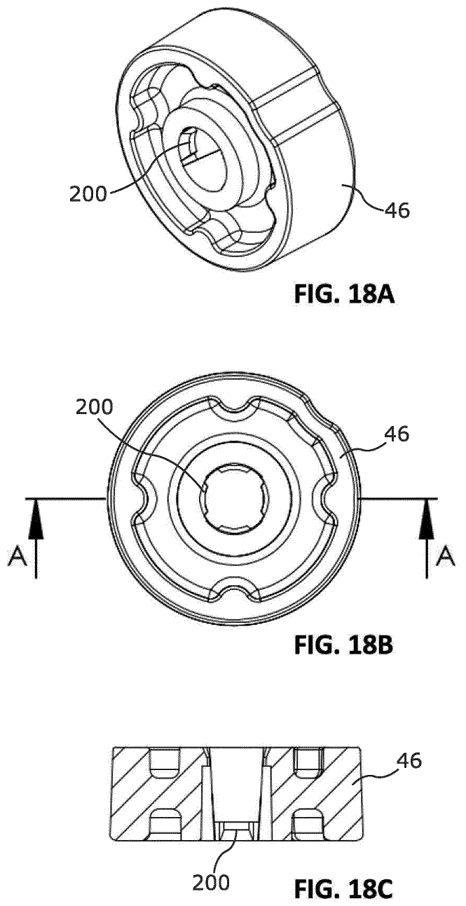

The piston rod 44, at its proximal end, forms coupling means or element 198 in the form of an undercut that engage with coupling means or element 200 in the form of radially inwardly extending ribs on an inner circumferential surface of the piston disc 46 (cf. FIG. 18A-18C).

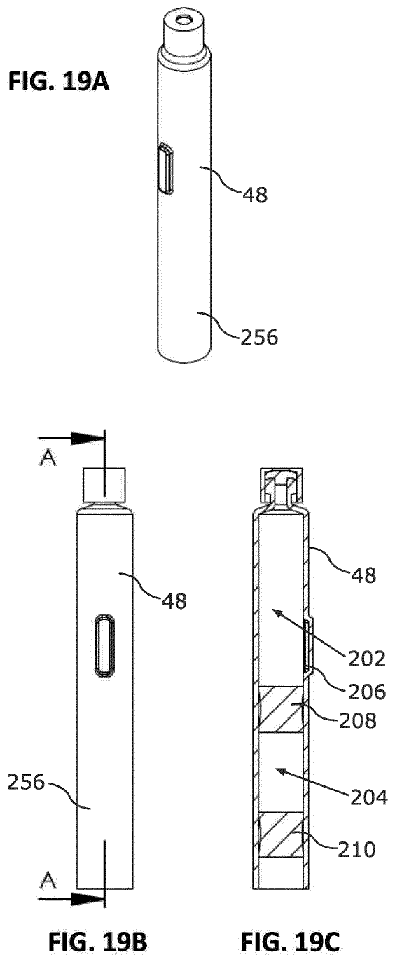

FIGS. 15A to 22B depict parts of a drug mixing or reconstitution unit 56 configured to mix different components, usually a lyophilized drug and a liquid solvent, to form an injectable liquid drug. In FIGS. 19A to 19C, the dual chamber cartridge 48 is shown. The dual chamber cartridge 48 is made of a transparent material such as glass. As can be seen from FIG. 19C, the cartridge 48 forms a first chamber 202 and a second chamber 204. In the as-delivered state shown in FIG. 19C, the first chamber 202 being arranged in proximal to the second chamber 204 comprises a bypass 206. The first chamber 202 and the second chamber 204 are separated by a first sealing element 208, e.g. made of a rubber material, that is axially slid ably arranged inside the dual chamber cartridge 48. In other words, the first sealing element 208 forms a distal end of the first chamber 202 and a proximal end of the second chamber 204. A second sealing element 210, e.g. made of a rubber material, forms a distal end of the second chamber 204. The second sealing element 210 can also be called a plunger. The piston disc 46 abuts the distal end face of the second sealing element 210 during mixture of the two components.

In the as-delivered state the lyophilized drug is in the first chamber 202 and the solvent in the second chamber 204.

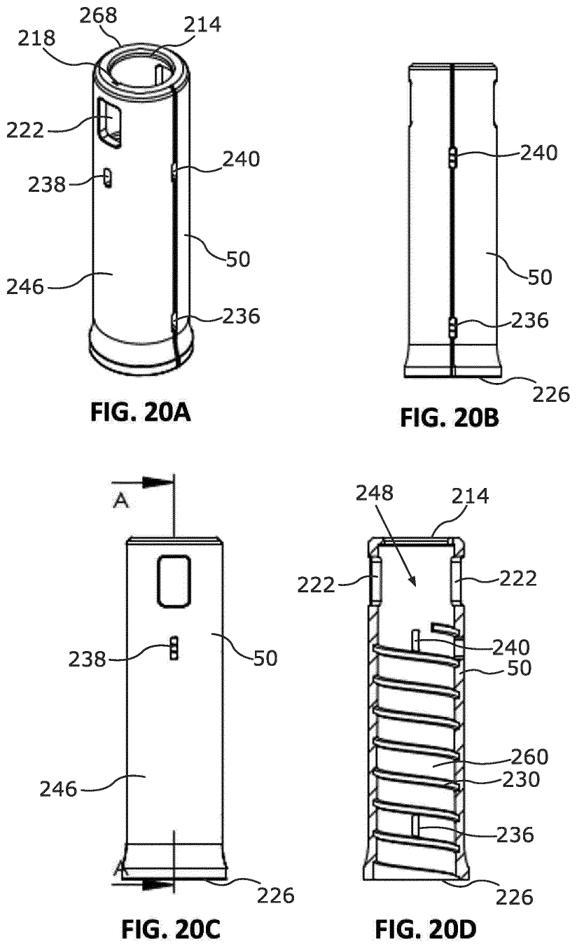

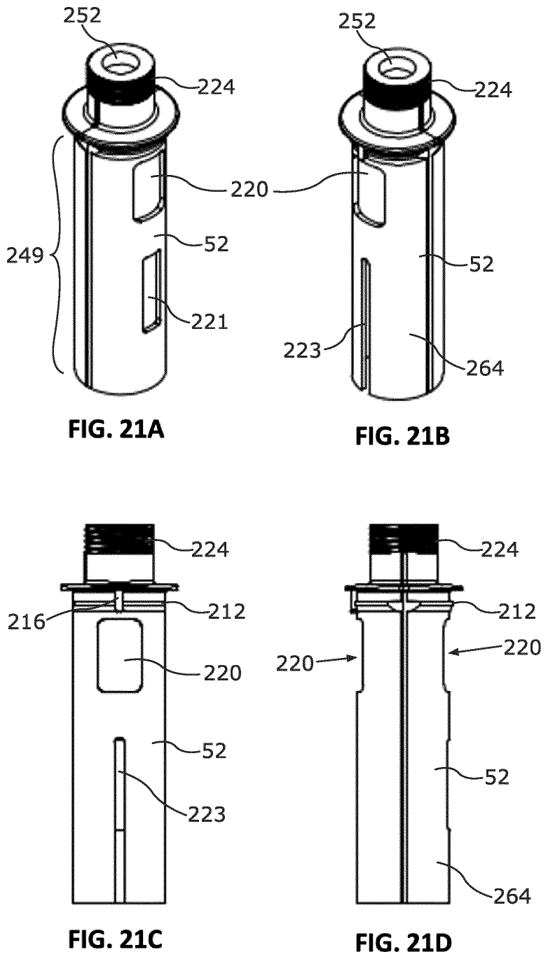

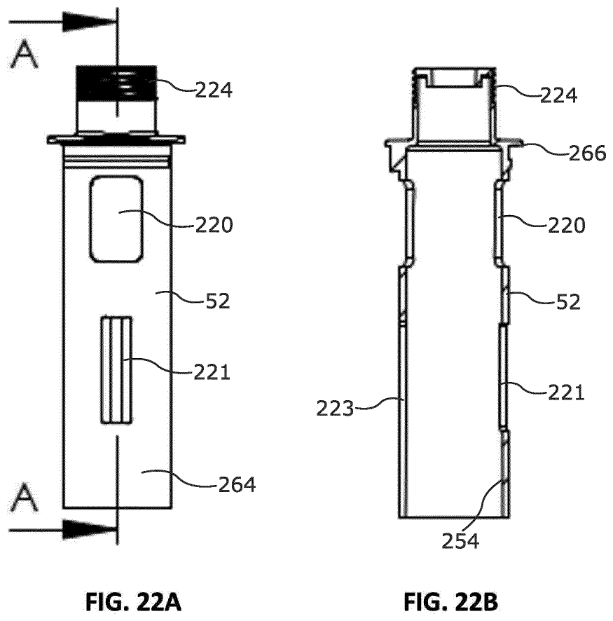

The dual chamber cartridge 48 is stored in the cartridge key 52 (cf. FIG. 21A-22b). The cartridge key 52 is axially and rotationally fixed to the cartridge container 50. To achieve that, the cartridge key 52 forms axial fixation means or element 212 in the form of a circumferentially extending groove that engage with axial fixation means or element 214 (cf. FIG. 20A) in the form of a circumferentially extending rib on an inner circumferential surface of the cartridge container 50. Furthermore, the cartridge key 52 forms rotation fixation means or element 216 in the form of a radially extending rib that engage rotation fixation means or element 218 in the form of a radially extending groove on the inner circumferential surface of the cartridge container 50. When the cartridge key 52 is attached to the cartridge container 50, an annular end face 266 (FIG. 22B) is in aerial abutment with a corresponding annular end face 268 (FIG. 20A) of the cartridge container 50. Both annular end faces 266 and 268 can be arranged perpendicular to the longitudinal axis of the pen 10. Furthermore, when the cartridge key 52 is attached to the cartridge container 50, a window 220 formed in the cartridge key 52 is aligned with a window 222 in the cartridge container 50 so that the patient can see the drug inside the transparent dual chamber cartridge 48 during reconstitution. At the proximal end of the cartridge key 52, which also defines the proximal end of the injection pen 10, a thread 224 is formed for attaching a needle (not shown). The thread 224 surrounds an opening 252 at a proximal end of the cartridge holder 52. The opening 252 is configured to receive a needle that is in fluid connection with an interior of the cartridge 48 inserted into the cartridge holder 52.

The cartridge key 52 forms an inner surface 254 (FIG. 22B) that defines a cylindrical receptacle that receives the cartridge 48 and prevents tilting of the cartridge 48 with respect to the longitudinal axis. Tilting of the cartridge 48 is prevented by an areal contact between a cylindrical outer surface 256 of the cartridge and the inner surface 254 in a holding section 249 of the cartridge holder 52. Furthermore, the cartridge holder 52 forms a cut-out 221 to receive the bypass 206 of the dual chamber cartridge 48. The bypass 206 form-fittingly engages the cut-out 221 so that the dual chamber cartridge 48 is axially and rotationally fixed to the cartridge key 52. On the opposite side of the cut-out 221, a slot 223 is formed extending in the axial direction. The slot 223 allows to reversibly widen the cartridge key 52 to axially insert the dual chamber cartridge 48 with the bypass 206.

In order to mix the different components in the dual chamber cartridge 48 and to prime the injection pen 10, the cartridge container 50 is screwed onto the piston rod guide 42 until a distal end surface 226 of the cartridge container 50 abuts a proximal surface 228 (cf. FIG. 15B) of the piston guide 42. In order to do so, the cartridge container 50 forms an outer surface 246 that can be grasped with one hand to screw the cartridge container 50 onto the piston rod guide 42. When the cartridge container 50 is screwed onto the piston rod guide 42, a proximal portion of the piston rod guide 42 enters an annular space 258 (FIG. 23B) between an outer circumferential surface 264 of the cartridge holder 52 and an inner circumferential surface 260 of the cartridge container 50 defining an inner space 248 of the cartridge container 260. In order to screw the cartridge container 50 onto the piston rod guide 42, a first thread 230 is formed on the inner circumferential surface 260 of the cartridge container 50 that is engaged with a second thread 232 formed on the outer circumferential surface 262 of the piston rod guide 42. As can be seen in FIG. 15A, the piston rod guide 42 forms a snap element 234. The snap element 234 allows screwing, i.e. a compulsory guided combined axial and rotational movement, of the cartridge container 50 relative to the piston rod guide 42 in the distal direction but blocks screwing of the cartridge container 50 relative to the piston rod guide 42 in the proximal direction 1 if the snap element 234 engages with one of the openings 236, 238, and 240. The first opening 236 (cf. FIG. 20B) is configured to define a starting position of the cartridge container 50 and makes sure that the cartridge container 50 cannot be detached from the piston rod guide 42. This starting position or as-delivered state is shown in FIGS. 25A and 25B.

The second opening 238 defines a reconstitution state of the cartridge container 50. In this state, the second chamber 202 still contains air so that the injection pen 10 can be moved forth and back to ensure that the drug is homogenously mixed together. The second opening 238 can be omitted. Therefore, the present disclosure is also directed at an embodiment of the injection pen 10 that features the first 236 and third opening 240 but not the second opening 238. The third opening 240 defines a knob cover unfastening state of the cartridge container 50 where the most of the air is expelled from the second chamber 202, which now contains the reconstituted medicament ready for use.

In the following with regard to FIGS. 23A to 33B, different states of the injection pen 10 are described during usage of the pen 10.

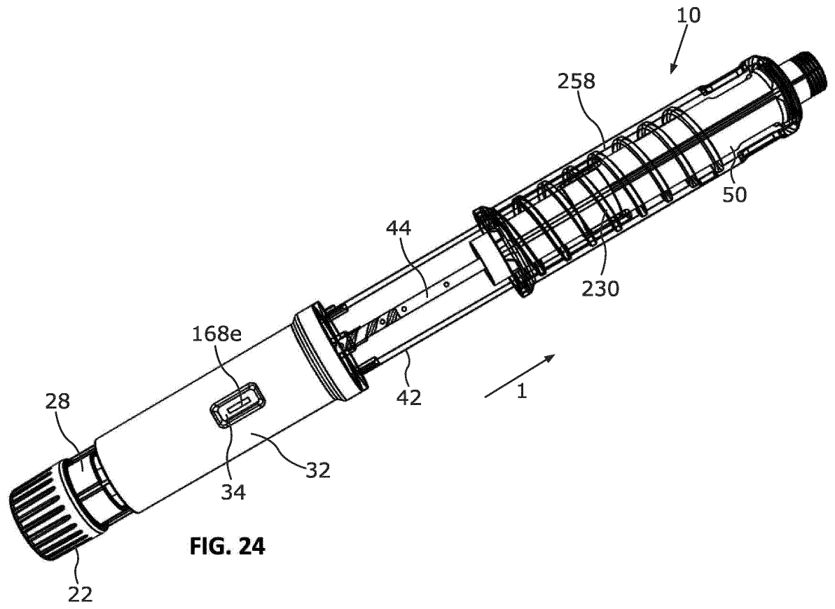

FIGS. 23A to 25B depict the injection pen 10 in the as-delivered state. As can be seen in FIG. 23A, the knob cover 16 covers a distal end section of the injection pen 10 up to a joint between the housing 32 and the piston guide 42. Therefore, the dose setting knob 22 is fully covered by the knob cover 16 so that it is not possible for the user to prematurely set a dose in this state. Looking at FIG. 23B, it can be seen that in the as-delivered state, the drug reconstitution unit 56 forms two separate chambers 202, 204 divided by the first sealing element 208. That means that the two components of the drug, each being stored in one of the two chambers 202, 204 are not yet mixed together. As can be seen in FIG. 24, where the knob cover 16 is blanked out to show what is under the knob cover 16, the dose setting sleeve 34 indicates that the injection pen 10 is in a preset state which differs from a zero-dose state.

Accordingly, the dose setting knob 22 is also in a preset position differing from a zero-dose position. As can be seen in FIGS. 25A and 25B, the snap element 234 of the piston rod guide 42 is snapped into the first opening 236 of the cartridge container 50. In FIG. 24, the cartridge container 50 is depicted as semi-transparent in order to show the first thread 230 formed on the inner circumferential surface of the cartridge container 50. Secondly, the piston rod guide 42 is also depicted as semi-transparent to show the position of the piston rod 44 in the preset state.

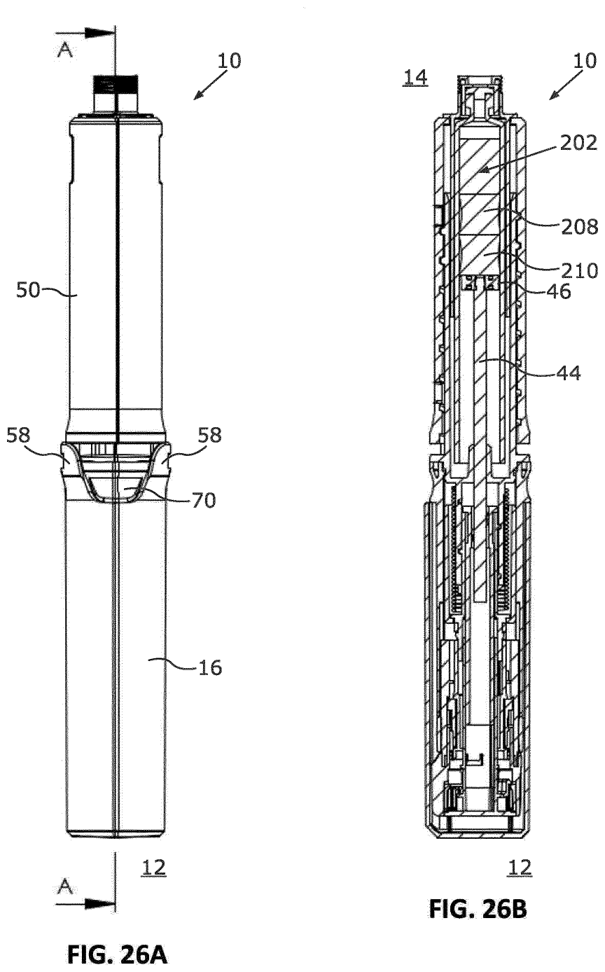

To start preparation of the drug, as can be seen from comparing FIGS. 25A and 26A, the cartridge container 50 is rotated by the user which causes the cartridge container 50 including the cartridge key 52 and the dual chamber cartridge 48 to move in the distal direction relative to the piston rod guide 42. The piston rod guide 42 thereby moves into the annular space 258 (FIG. 23B) between the cartridge container 50 and the cartridge holder 52. The piston disc 46 is snapped to the piston rod 44, which is rotationally fixed by the piston rod guide 42 and axially fixed by the nut 38. The piston disc 46 thus blocks the movement of the second sealing element 210 arranged in the dual chamber cartridge 48 so that the second sealing element 210 slides along the inner circumferential surface of the dual chamber cartridge 48 while the cartridge container 50 is further screwed onto the piston rod guide 42. The solvent stored in the second chamber 204 pushes against the first sealing element 208 which also causes the first sealing element 208 to slide along the inner circumferential surface of the dual chamber cartridge 48. This would cause an overpressure in the cartridge, but the air can escape through the double-ended needle the user attached to thread 224. When the first sealing element 208 reaches the bypass 206 (cf. FIG. 23B), the first chamber 202 and the second chamber 204 are connected by the bypass 206 and therefore, the lyophilized drug stored in the first chamber 202 and the solvent stored in the second chamber 204 mix.

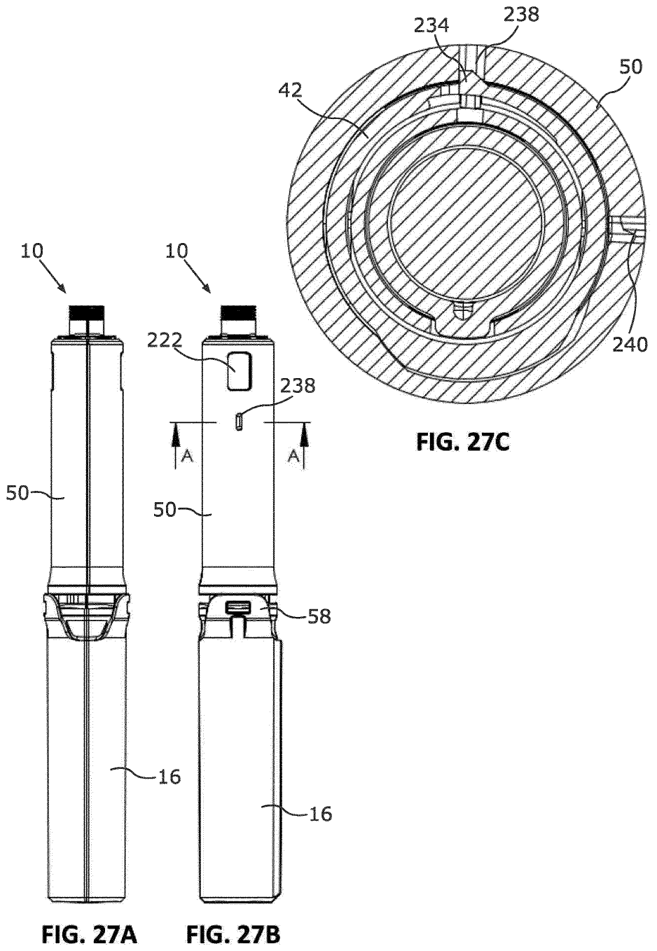

In the reconstitution state shown in FIGS. 26A to 27C, the mixed drug is stored in the first chamber 202 between the first sealing element 208 and the proximal end 14 of the dual chamber cartridge 48. As can be seen in FIG. 26B, a proximal end surface of the second sealing element 210 abuts a distal end surface of the first sealing element 208 so that no second chamber 204 is present anymore in the reconstitution state. As can be seen in FIG. 27C, the snap element 234 of the piston rod guide 42 is snapped into the second opening 238 of the cartridge container 50. In this state, the front chamber 202 still contains a significant amount of air, which helps to create turbulence when moving the pen, so that the mixing of the lyophilized drug is easier. As mentioned before, the second opening 238 can be omitted. In that case the mixing takes place with a low residual amount of air.

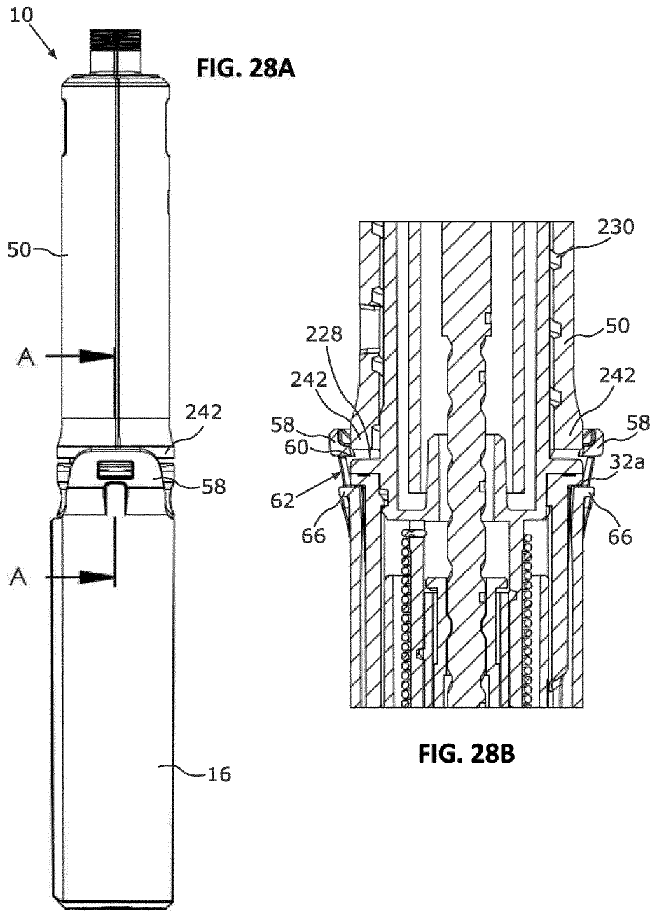

After the reconstitution of the drug is finished, the cartridge container 50 is further rotated by the user causing the cartridge container 50 to move further axially in the distal direction relative to the piston rod guide 42. This causes a displacement section 242 positioned at a distal end of the cartridge container 50 to engage with and spread the wings 58 of the knob cover 16 radially outwardly (cf. FIG. 28B). Spreading the wings 58 radially outwardly causes the form-fitting engagement means 60 of the knob cover 16 to disengage from the coupling surface 228 so that the knob cover 16 is axially movable relative to the housing 32. It is now possible to pull off the knob cover 16 from the housing 32 in the distal direction resulting in the state shown on FIGS. 29A and 29B. When the cartridge container 50 is fully screwed onto the piston rod guide 42, a radial end stop 244 formed on the outer circumferential surface 262 of the piston rod guide 42 abuts a radial end stop (not shown) on an inner circumferential surface of the cartridge container 50. Furthermore, the snap element 234 of the piston rod guide 42 is snapped into the third opening 240. Consequently, the cartridge container 50 is rotationally locked to the piston rod guide 42 and the housing 32 of the device. Therefore, movement of the cartridge container 50 and the cartridge 48 respect to the housing 32 and the piston rod guide 42 is inhibited.

As can be seen in FIGS. 29A and 29B, at this stage the knob key 30 is still clipped onto the outer circumferential surface of the dose selector 28 between the proximal edge 160 of the dose setting knob 22 and the distal edge 162 of the housing 32. The knob key 30 can be taken away from the dose selector 28 only after the knob cover 16 has been removed by slightly bending the knob key 30.

FIGS. 29A and 29B depict the dose delivery mechanism 54 with all remaining components of the dose delivery mechanism 54 except for the piston rod 44 and components axially fixed to the piston rod 44 being axially located in their zero dose position with respect to the housing 32 along the longitudinal axis of the dose delivery mechanism 54. The remaining components are in their respective zero dose position prior to setting a dose with the dose delivery mechanism 54. The piston rod 44 does not comprise a defined zero dose position with respect to the housing 32, since it moves in the proximal direction 1 during each injection. Therefore, the axial position of the piston rod 44 with respect to the housing 32 differs at the end of each injection. Furthermore, also the rotational position of the piston rod 44 with respect to the housing 32 can differ at the end of different injections.

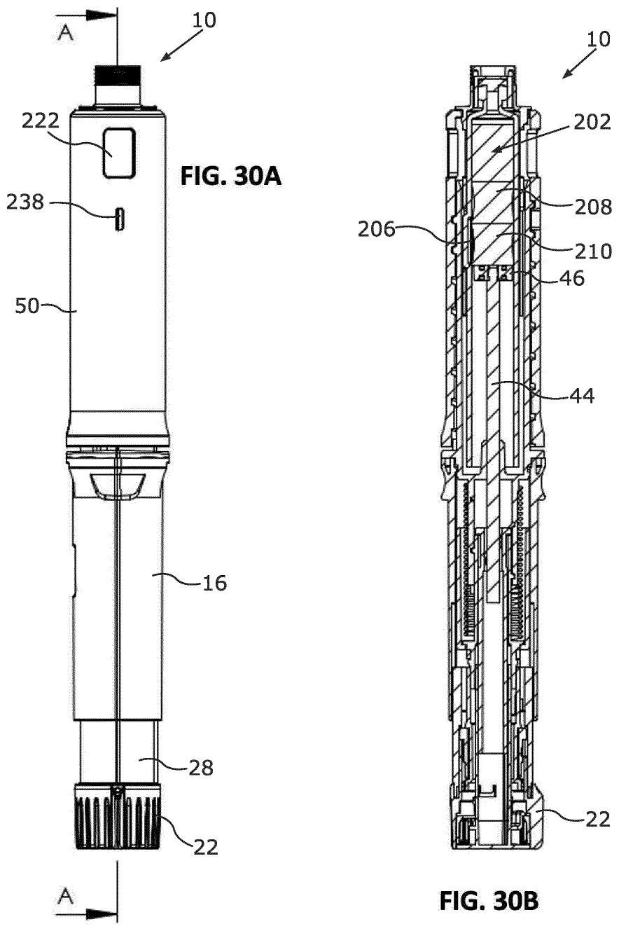

Afterwards, as can be seen when comparing FIGS. 29A and 30A, the dose setting knob 22 is rotated by the user the set a desired dose out of multiple possible settable doses. In this example, the dose setting knob 22 is rotated 180° to set the desired dose. While the dose setting knob 22 is rotated, the dose setting knob 22 makes a compulsory guided combined axial and rotational movement, namely a screw movement, in the distal direction.

Rotating the dose setting knob 22 causes rotation of the injection button 18, that is axially and rotationally connected to the dose setting knob 22 via the snap ring 20, the snap element 24, which is rotationally connected to the dose setting knob 22 via the teeth 108 intermeshing with the teeth 110, the driver 36, which is rotationally and axially coupled to the snap element 24, and the dose setting sleeve 34 which is rotationally and axially coupled to the driver 36. Rotation of the driver 36 causes the driver 36 to move axially in a distal direction due to the engagement of the outer thread 170 of the driver 36 and the inner thread 172 of the piston rod guide 42. The axial movement of the driver 36 causes the snap element 24 to move in a distal direction which pushes the injection button 18 and the dose setting knob 22 in the distal direction via the couplings means 102 of the snap element 24 interacting with the assembling means 98 of the injection button 18. This causes the dose setting knob 22 to perform a compulsory guided combined axial and rotational movement during dose setting.

Furthermore, rotating the dose setting knob 22 causes rotation of the injection button 18 that is rotationally coupled to the nut 38. Since the piston rod 44 is rotationally fixedly coupled to the piston rod guide 42 due to their corresponding out of round cross-sections 186, 188, the nut 38 moves in the distal direction when the dose setting knob 22 and therefore the nut 38 is rotated.

The amount of axial movement of the nut 38 relative to the piston rod 44 and the driver 36 relative to the piston guide 42 depends on the pitch of the respective thread. The outer thread 170 of the driver 36 has a greater pitch than the outer thread 190 of the piston rod 44 so that the driver 36 moves in the distal direction more than the nut 38. For example, the outer thread 170 of the driver 36 can have a pitch of 10.71 mm and the outer thread 190 of the piston rod 44 can have a pitch of 10.21 mm.

When the desired dose is set, the spiral torsion spring 40 applies a torque to the snap element 24 via the driver 36 to bring the dose definition element 116 in abutment with the respective dose stop 118a to 118d, namely with its side surface 122b. Due to the spring 40, the injection pen 10 is configured to rotationally self-align the snap element 24 and the dose selector 28 in different predefined rotational positions defining predefined doses.

If the user then pushes the injection button 18 on the distal end 12 of the injection pen 10, the dose setting knob 22 moves in the proximal direction 1 relative to the snap element 24. This results in the coupling means 100 being bend while passing the circumferential ledge 102 causes a counterforce in the distal direction which has to be overcome by the user to start the injections process. The dose setting knob 22 moving in the proximal direction 1 relative to the snap element 24 also results in the teeth 108 of the dose setting knob 22 disengaging with the teeth 110 of the snap element 24 and instead the teeth 108 of the dose setting knob 22 engaging with the teeth 114 of the connector 26. Since the connector 26 is rotationally coupled to the housing 32 via the dose selector 28, the dose setting knob 22 is rotationally fixed to the housing 32. Therefore, during dose delivery, the dose setting knob 22, the injection button 18, the dose selector 28, and the nut 38 do not rotate relative to the housing 32.

If the user further pushes injection button 18, the injection button 18 and the dose selector 28 move relative to the snap element 24 in the proximal direction 1. Thereby, the dose definition element 116 of the snap element 24 passes through the circumferentially extending rib 156 on the dose selector 28 through the respective cut-out 158a-158d corresponding to the set dose. At the same time, the hard stop 126 of the dose selector 28 moves in the axial direction relative to the hard stop 124 on the snap element 24 which allows the dose selector 28 and the snap element 24 to rotate relative to each other past the pre-set dose position towards the zero-dose position.

When the injection button 18 is pushed during dose delivery, the injection button 18 pushes the driver 36 via the snap element 24 in the proximal direction 1. The spring 40 supports the axial movement of the driver 36 by applying a torque to the driver 36 resulting in an axial movement of the driver 36 in the proximal direction 1 due to the outer thread 170 of the driver 36. The driver pushes the nut 38 in the proximal direction 1 which causes the piston rod 44 to move in the proximal direction 1. The movement of the piston rod 44 and the piston disc 46 in the proximal direction 1 causes the drug to be injected into the patient. Since the injection pen 10 is made to inject relatively large amounts of drug, the pen 10 does not have a so-called gearing. In other words, the parts that are configured to rotate relative to the housing during dose delivery are connected to the housing 32. This means that the distance the piston disc 46 advances is essentially equal to the distance the injection button 18 is pushed in the proximal direction 1 relative to the housing 32.

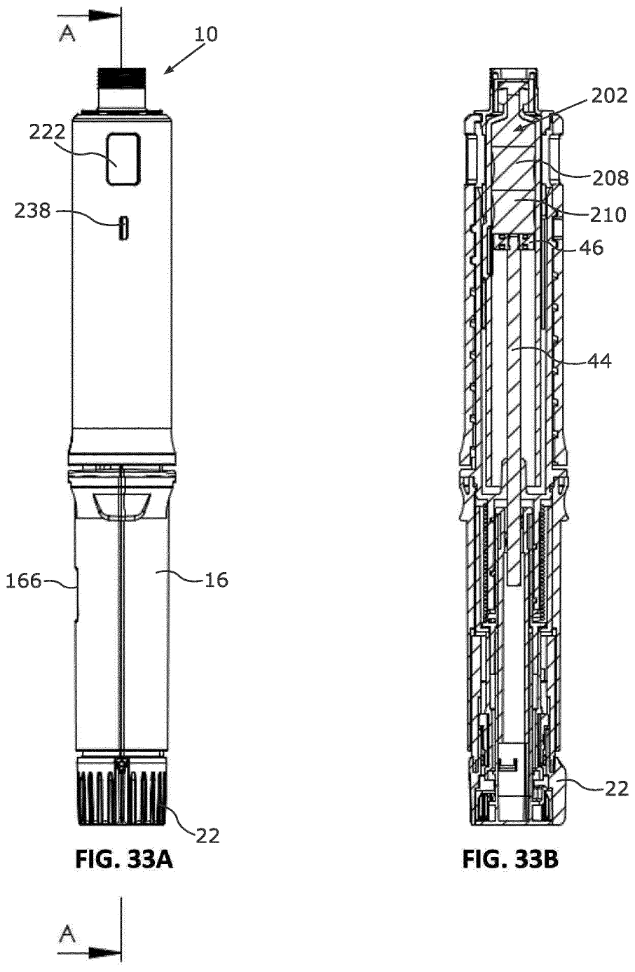

Since the driver rotates relative to the housing due to its outer thread 170, the dose setting sleeve 34 rotates during dose delivery. At the end of the dose delivery (cf. FIGS. 33A and 33B) the dose setting sleeve 34 is in a rotational position in which a zero-dose label can be seen through the window 166 of the housing 32. The end of dose stop 174 (cf. FIG. 13B) of the driver 36 and the end of dose stop 176 (cf. FIG. 16C) of the piston rod guide 42 define an end of the movement of the injection button 18 in the proximal direction 1 during dose de-livery.

At the end of the dose delivery, the coupling means 100 on the injection button 18 passes the coupling means 102 of the snap element 24 when initiating the injection, which permanently rotationally couples the dose setting knob 22 and the injection button 18 to the housing 32. Thus, the injection pen 10 is rendered inoperable, as the user cannot rotate the dose setting knob 22 to set a new dose.

The injection pen 10 allows for adjusting an axial position of the piston rod 44 with respect to the housing 32 in the preassembled state of the dose delivery mechanism 54. In the preassembled state, the injection button 18, which forms an adjusting element 18 of the dose delivery mechanism 54, engages with its distal assembling means 98a with the coupling means 102 of the snap element 24. This allows to position the injection button 18 in a more distal preassembled position compared to its assembled position in an assembled state of the dose delivery mechanism 54, in which assembled position the adjusting element 18 engages the coupling means 102 with its proximal assembling means 98b.

In the preassembled position, the adjusting element 18 protrudes from the dose setting element 22 and is free to rotate with respect to the dose setting element 22. Rotation of the adjusting element 18 then rotates the nut 38 with respect to the piston rod 44 and thereby causes axial movement of the piston rod 44 due to the threaded connection 189 between the piston rod 44 and the nut 38.

Adjustment of the piston rod 44 in the preassembled state is further detailed below in connection with a second injection pen 330 according to the present disclosure, which is a variant of the injection pen 10 shown in the previous figures.

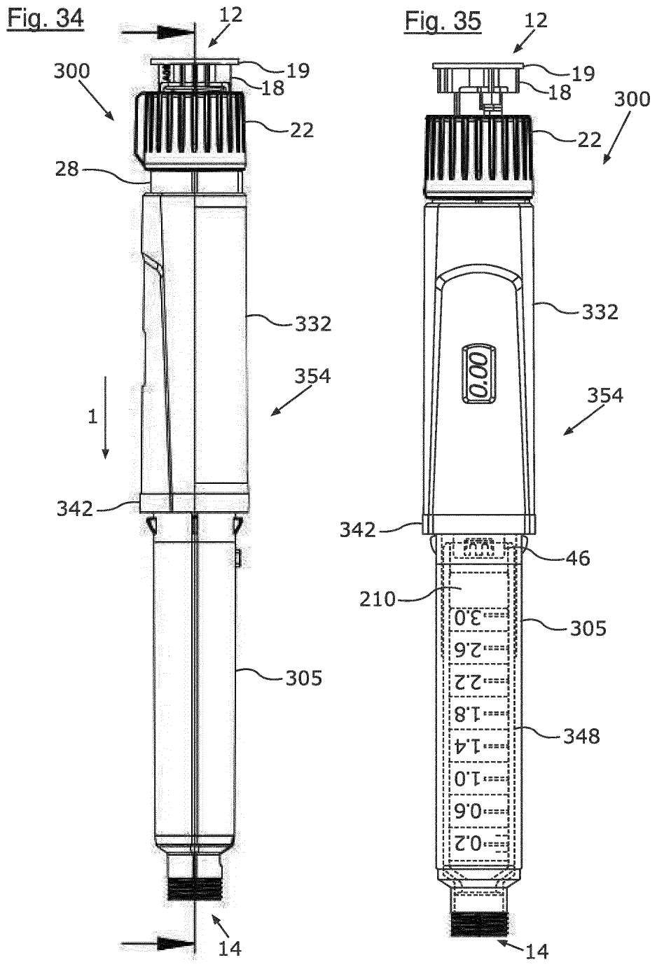

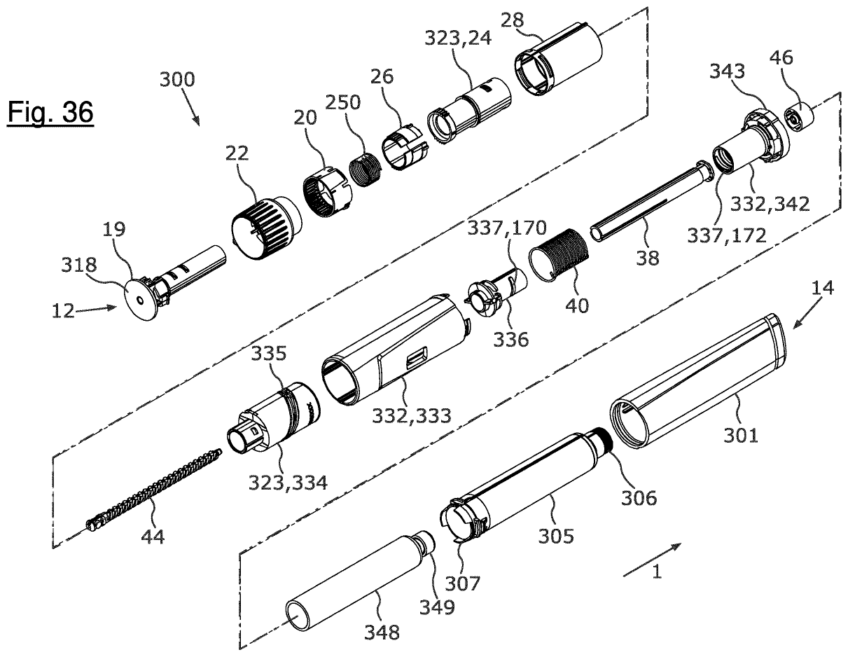

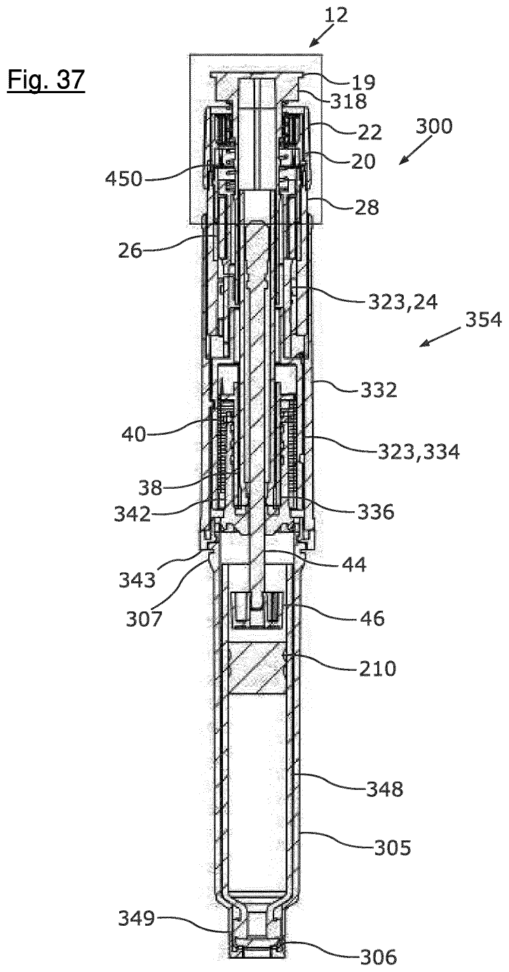

FIGS. 34 and 35 show the second injection pen 300 in the preassembled state, FIG. 36 shows an exploded view of the second injection pen and FIG. 37 shows a longitudinal cut through the second injection pen 300 in the preassembled state. As far as no differences are disclosed in the description or the Figures, the second injection pen 300 is configured as it is disclosed for the injection pen 10 of the previous Figures and vice versa.

The second injection pen 300 comprises a dose delivery mechanism 354. As far as no differences are disclosed in the description or the Figures, the dose delivery mechanism 354 of the second injection pen 300 is configured as it is disclosed for the dose delivery mechanism 54 of the injection pen 10 and vice versa.

The dose delivery mechanism 354 comprises a housing 332 that has an upper housing part 333 and a piston rod guide 342 that forms a lower housing part. The upper housing part 333 and the piston rod guide 342 are rigidly connected to each other via a form-fit connection. In particular, the upper housing part 333 and the piston rod guide 342 are axially and rotationally fixed to each other. The lower housing part formed by the piston rod guide 342 is configured to connect to a medicament container holder 305 that receives a medicament container 348. The medicament container holder 305 comprises a connector 307 that is located at a distal end of the medicament container holder 305. The connector 307 is configured to connect to a corresponding connector 343 of the piston rod guide 342, the corresponding connector 343 being accessible at a proximal side of the piston rod guide 342. The connectors 307, 343 provide a non-releasable form-fit connection between the medicament container holder 305 and the housing 332 after attachment of the medicament container holder 305 to the housing 332.

The medicament container 348 has a single medicament chamber that is sealed by a single plunger 210 at its distal end (see FIG. 35). The medicament chamber contains a fluid medicament. At its proximal needle end 349, the medicament container 348 comprises a septum that is configured to be punched upon attaching a double-sided cannula to a needle connector 306 located at the proximal end of the medicament container holder 305. A cap 301 is releasably attachable to the medicament container holder 305 during storage of the injection pen 300.

The dose delivery mechanism 354 comprises an injection button that constitutes an adjusting member 318, a snap element 24, a dosing element 334 and a driver 336. In the preassembled state and in the assembled state of the injection pen 300, the snap element 24 and the dosing element 334 are rigidly connected to each other and form a dosing member 323 of the dose delivery mechanism 354. The dosing element 334 is coupled to a housing 332 of the dose delivery mechanism 354 via a threaded connection 335. The threaded connection 335 comprises an outer thread on an outer surface of the dosing member 323 and an inner thread (not visible in FIG. 36) on an inner surface of the housing 332. With other embodiments, the dosing member 323 can also be configured as a single component.

The dosing member 323 constitutes a dose indication member of the dose delivery mechanism 354. Thereby, the dosing element 334 comprises markings that are visible through a window in the upper housing part 333 of the housing 332 upon rotation of the dosing member 323 with respect to the housing 332 during dose setting.

The driver 336 is connected to the housing 332 via a further threaded connection 337 that acts between the driver 336 and the piston rod guide 342, as it is described for the driver 36 and the piston rod guide 42 of the injection pen 10. The driver 336 is furthermore rotationally fixed and axially movable with respect to the dosing member 323 via a splined connection. Thereby, the driver 336 is received within the dosing element 334 of the dosing member 323. The splined connection comprises first spline elements on the outer circumference of the driver 336 that engage with corresponding second spline elements on the inner circumference of the dosing element 334. Simultaneous rotation of the driver 336 and the dosing member 323 requires axial movement of the driver 336 due to the further threaded connection 337 to the housing 332 and simultaneous axial movement of the dosing member 323 due to the threaded connection 335 to the housing 332.

A pitch of the threaded connection 335 between the dosing element 334 and the housing 332 deviates from a pitch of the further threaded connection 337 between the driver 336 and the housing 332. A ratio of these pitches defines a mechanical advantage of the dose delivery mechanism 354 during dose delivery and a forced proximal movement of the dosing member 334 by a first axial distance leads to a proximal movement of the driver 336 by a second axial distance that deviates from the first axial distance.

FIG. 37 depict the dose delivery mechanism 354 with all components of the dose delivery mechanism 354 except for the piston rod 44 and components axially fixed to the piston rod 44 being axially located in their zero dose position with respect to the housing 332. The piston rod 44 does not comprise a defined zero dose position with respect to the housing 32, since it repeatedly moves in the proximal direction 1 during each injection. Therefore, the axial position of the piston rod 44 with respect to the housing 32 differs at the end of each injection. Furthermore, also the rotational position of the piston rod 44 with respect to the housing 32 can differ at the end of different injections.

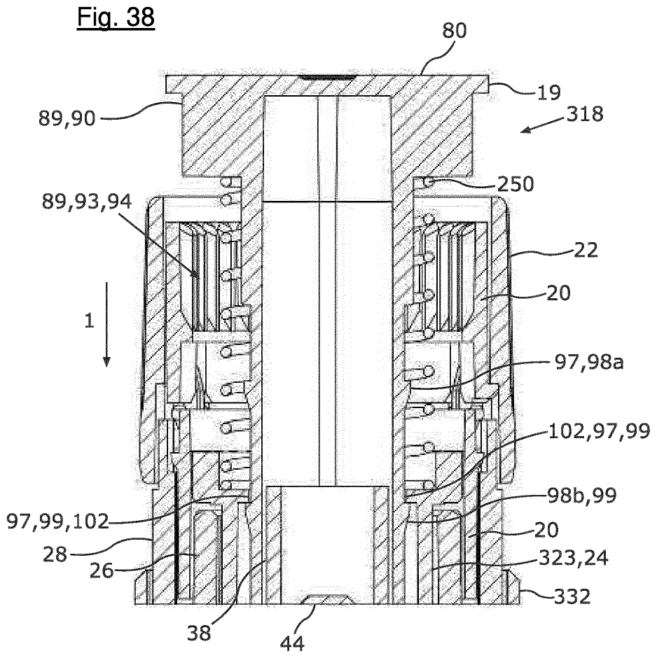

FIG. 38 shows a detailed view of a distal portion of the second injection pen 300 in the preassembled state. The adjusting element 318 deviates from the adjusting element 18 of the injection pen 10 in that it does not feature the coupling means 100 to axially lock the adjusting element 18 to the snap element 24 upon dose delivery. The second injection pen 300 therefore allows to repeatedly set and inject user definable doses. Apart from this modification, the adjusting element 318 is configured as it is disclosed for the adjusting element 18 and vice versa. In particular, the adjusting element 18 of the injection pen 10 is configured to adjust the position of the piston rod 46 in the preassembled state in the same way as it is disclosed in the following for the adjusting element 318 of the second injection pen 300. Furthermore, the adjusting element 318 forms an actuation member of the second injection pen 300. The actuation member is configured to be activated by a user to deliver a set dose. Exemplarily, the actuation member is configured to be pushed in the proximal direction by the user to deliver the set dose.

In the preassembled state shown in FIGS. 37 and 38, the proximal assembling means 98b of the adjusting element 318 engage with the coupling means 102 of the snap element 24 to allow to position the adjusting element 318 in a preassembled position with respect to the housing 332. The preassembled position is the most distal axial position of the adjusting element 318 that is reached when the assembling means 98b engage with the coupling means 102 of the snap element 24 upon distal movement of the adjusting element 318. A biasing element 250 in the form of a spring, which biasing element 250 acts between the snap element 24 and the adjusting element 318, biases the adjusting element 318 in the distal direction into the preassembled position. Since the injection pen 10 does not feature the biasing element 250, the adjusting element 18 of the injection pen 10 is not held in the preassembled position. With the injection pen 10, an assembler of the device manually positions the adjusting element 18 in the preassembled position.

The assembling means 98b form a latch part of a latching mechanism 99 and the coupling means 102 of the snap element 24 form a latch counterpart of the latching mechanism 99. Furthermore, the dosing member 323 with the snap element 24 forms a counter member of the latching mechanism 99. The latching mechanism 99 prevents detachment of the adjusting element 318 from the housing 332 in the preassembled state.

In the preassembled position, the adjusting element 318 distally protrudes from the dose setting element formed by the dose knob 22. The rotation fixation means 90 of the adjusting element 318 then do not engage the rotation fixation means 94 of the snap ring 20 so that the adjusting element 318 is rotationally movable with respect to the housing 332 and the dose setting element 22.

In the preassembled position, an outer rim 19 of the adjusting element 318 is accessible to an assembler of the injection pen 300. When rotating the adjusting element 318 with respect to the housing 332 and the dose setting element 22, the adjusting element 318 rotates the nut 38. The nut 38 thereby does not axially move with respect to the housing 332 since it is restrained by the stationary driver 336 pushing on the pressing surface 194 at the proximal end of the nut 38. The threaded connection 189 between the piston rod 44, which forms a first threaded element, and the rotating nut 38, which forms a second threaded element, then causes the piston rod 44 to axially move with respect to the housing 332. The second injection pen 300 thus allows to adjust the axial position of the piston rod 44 by rotating the adjusting element 318 with respect to the housing 332 and the dose setting element 22. The same holds for the injection pen 10, the injection button 18 of which also forms an adjusting element.

The dose delivery mechanisms 54, 354 of the injection pens 10, 300 each comprise a rotational lock 89, which is formed by the rotational fixation means 90 of the respective adjusting element 18, 318 and the toothed part 93 of the respective snap ring 20. The snap ring 20 thereby forms a connector between the respective adjusting element 18, 318 and the respective dose setting element 22 and the adjusting element 18, 318 is rotationally and/or axially fixed to the dose setting element 22 in the assembled state via the connector 20. Furthermore, the dose setting element 22 forms a counter element to which the adjusting element 18, 318 is attached in the assembled state of the respective dose delivery mechanism 54, 354.

Furthermore, the axial fixation means 82 of the adjusting elements 18, 318 and the rib 84 of the snap ring 20 each form an axial lock 81 that allows axial movement between the adjusting element 18, 318 and the dose setting element 22 in the preassembled state of the dose delivery mechanisms 54, 354 and that prevents axial movement between the adjusting elements 18, 318 and the dose setting element 22 in the assembled state.

The axial fixation means 82 of the adjusting elements 18, 318 and the rib 84 of the snap ring 20 also form a latching mechanism that acts between the adjusting elements 18, 318 and the counter element formed by the dose setting element 22. In the assembled state of the dose delivery mechanisms 54, 354, the latching mechanism blocks the movement of the adjusting elements 18, 318 from a second position with respect to the counter element into a first position with respect to the counter element. The second position thereby is the proximal position in which the adjusting elements 18, 318 are rotationally and axially fixed to the counter element and the first position is the distal position that the adjusting elements 18, 318 take up in the preassembled state and in which the adjusting elements 18, 318 are rotatable with respect to the counter element.

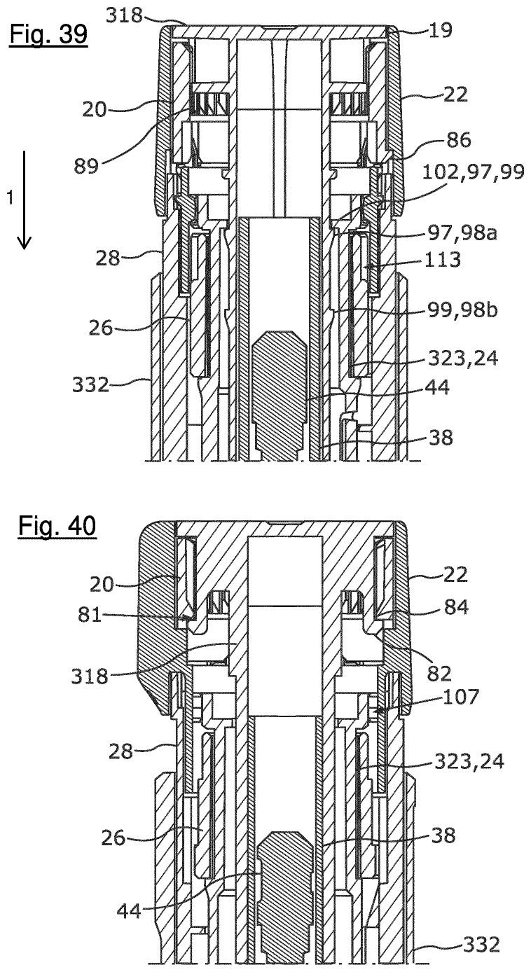

FIG. 39 shows a detailed view of a longitudinal cut through the distal end of the second injection pen 300 in the assembled state during dose setting and FIG. 40 shows a detailed view of a further longitudinal cut through the distal end of the second injection pen 300 in the assembled state during dose setting. Cut planes of the longitudinal cuts shown in FIGS. 39 and 40 are orientated perpendicular to each other.

The injection pen 300 is transferred from the preassembled state into the assembled state by proximally moving the adjusting element 318 from the preassembled position into an assembled position with respect to the dose setting element 22 and the housing 332. This rotationally and axially locks the adjusting element 318 to the dose setting element 22 via the connector 20, the rotational lock 89 and the axial lock 81. In the assembled state, the distal assembly means 98a of the adjusting element 318 engage with the coupling means 102 of the snap element 24 thus irreversibly blocking movement of the adjusting element 318 from the assembled position into the preassembled position.

The distal assembly means 98a of the adjusting element 318 forms a latch part of a latching mechanism 97 that is configured to prevent the adjusting element 318 from moving from the assembled position into the preassembled position with respect to the housing 332. The coupling means 102 of the snap element 24 forms a latch counterpart of the latching mechanism 97 and the dosing member 323 with the snap element 24 forms a counter member of the latching mechanism 97.

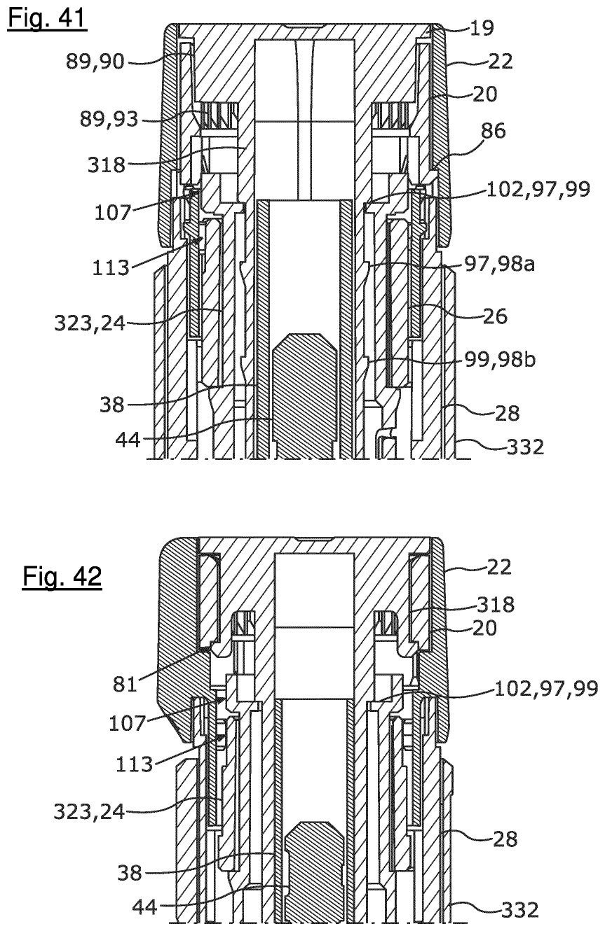

FIG. 41 shows a detailed view of a longitudinal cut through the distal end of the second injection pen 300 in the assembled state during dose delivery and FIG. 42 shows a detailed view of a further longitudinal cut through the distal end of the second injection pen 300 in the assembled state during dose delivery. Cut planes of the longitudinal cuts shown in FIGS. 41 and 42 are orientated perpendicular to each other. FIGS. 41 and 42 thereby show the injection pen 300 at the end of dose delivery when a set dose has been fully expelled and the adjusting element 318 is still pressed by a user of the injection pen 300 in the proximal direction 1.

During dose setting in the assembled state, the adjusting element 318 is rotationally coupled to the dosing member 323 via a clutch mechanism 107 formed by the teeth 108 on the inside surface of the dose setting element 22 (see FIG. 5) and the teeth 110 on the outside surface of the snap element 24 (see FIG. 6). The dose setting element 22 forms a first clutch member of the clutch mechanism 107 and the snap element 24 forms a second clutch member of the clutch mechanism 107. The dosing members 23, 323 of the dose delivery mechanisms 54, 354 each form respective further members of the dose delivery mechanisms 54, 354.

With each dose delivery mechanism 54, 354, the respective clutch mechanism 107 rotationally couples the respective adjusting element 18, 318 to the respective further member in a closed state of the respective clutch mechanism 107 during dose setting in the assembled state and rotationally decouples the respective adjusting element 18, 318 from the respective further member in an opened state of the respective clutch mechanism 107 during dose delivery in the assembled state.

With other embodiments of the clutch mechanisms 107 that couple the adjusting elements 18, 318 to the further members, the second clutch members can also be integrally formed with the further members. For example, when integrally forming the snap element 24 and the dosing element 334 as a single-piece dosing member 323, this dosing member 323 constitutes the further member and, at the same time, the second clutch member.

During dose delivery, the clutch mechanism 107 is opened thus rotationally de-coupling the adjusting element 318 and the dosing member 323. In the preassembled state, the clutch mechanism 107 is closed but the adjusting element 318 is rotationally decoupled from the clutch mechanism 107 so that the clutch mechanism 107 does not transfer rotation of the adjusting element 318 to the dosing member 323. In particular, the adjusting element 318 is rotationally decoupled from both the dose setting element 22 and the dosing member 323 in the preassembled state.

During dose delivery in the assembled state, the adjusting element 318 is rotationally coupled to the housing 332 via a further clutch mechanism 113, whereby the further clutch mechanism 113 is formed by the teeth 108 on the inside surface of the dose setting element 22 and the teeth 114 located on the outside surface of the connector 26. The dose setting element 22 thereby forms a first clutch member of the further clutch mechanism 113 and the connector 26 forms a second clutch member of the further clutch mechanism 113. If the further clutch mechanism 113 is in a closed state and the first clutch member engages with the second clutch member, the adjusting element 318 is rotationally fixed to an additional member, the additional member being formed by the housing 332.

During dose setting, the further clutch mechanism 113 is opened so that the adjusting element 318 is allowed to rotate with respect to the housing 332. During dose delivery, the further clutch mechanism 118 disclosed so that the adjusting element is rotationally fixed with respect to the housing. In the preassembled state, the adjusting element 318 is rotationally decoupled from the further clutch mechanism 113, since it is allowed to rotate with respect to both the first clutch member formed by the dose setting element 22 and the second clutch member formed by the connector 26.

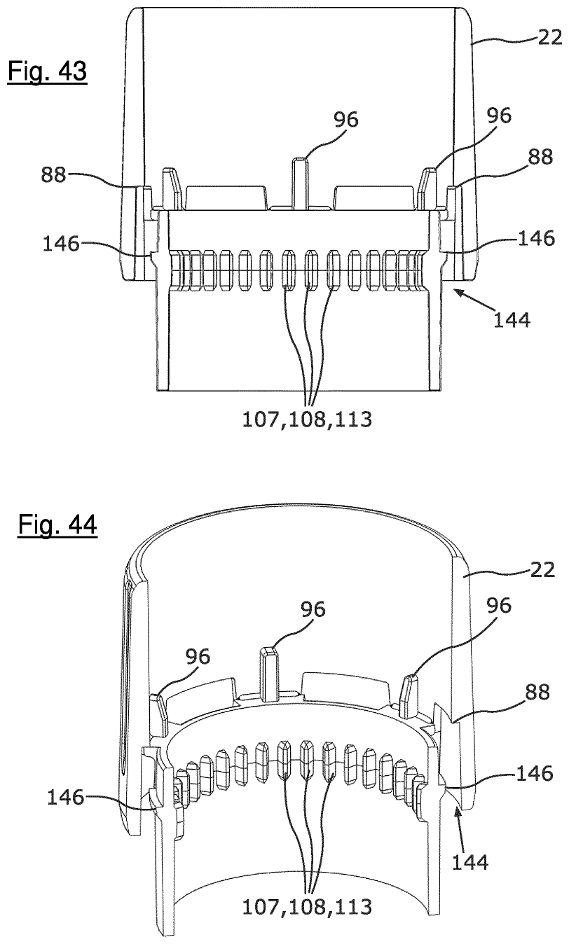

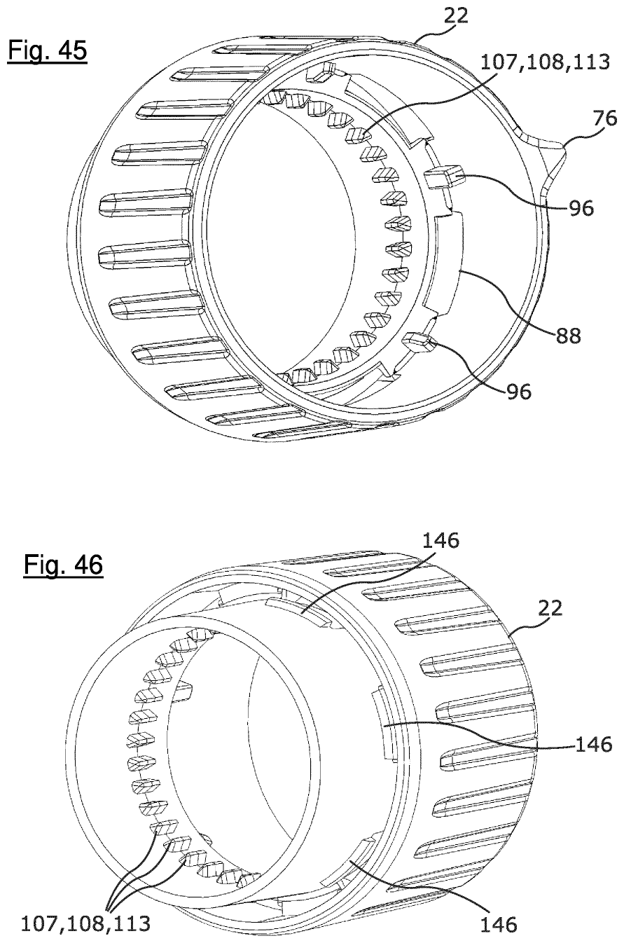

FIG. 43 shows a longitudinal cut through the dose setting element 22 of the injection pen 10 and the second injection pen 300, FIG. 44 shows a perspective view of the longitudinal cut through the dose setting element 22, FIG. 45 shows a perspective distal view of the dose setting element 22 and FIG. 46 shows a prospective proximal view of the dose setting element 22 with the teeth 108 of the clutch mechanisms 107, 113.

With both the clutch mechanism 107 and the further clutch mechanism 113, the clutch mechanisms 107, 113 are in a closed state during one of dose setting and dose delivery and the clutch mechanisms 107, 113 are in an opened state during the other one of dose setting and dose delivery. The clutch mechanism 107 thereby is closed when the further clutch mechanism 113 is opened and the clutch mechanism 107 is opened when the further clutch mechanism 113 is closed.

Furthermore, with each clutch mechanism 107, 113, the adjusting element 318 takes up a dose setting position with respect to the respective second clutch member 24, 26 during dose setting and it takes up a dose delivery position with respect to the respective second clutch member 24, 26 during dose delivery. The dose delivery position thereby is axially shifted with respect to the dose setting position. Exemplarily, the dose delivery position is axially shifted in the proximal direction 1.

With the dose delivery mechanisms 54, 354, the further clutch mechanism 113 also forms a locking mechanism that is configured to rotationally lock the adjusting element 18, 318 to the housing 32, 332 during dose delivery in the assembled state.

The dose selectors 28 of the dose delivery mechanisms 54, 354 each form a retaining member of the respective dose delivery mechanism 54, 354. Each adjusting element 18, 318 is located in a first axial position with respect to the retaining member in the preassembled state and each adjusting element 18, 318 is transferred from the first axial position into a second axial position with respect to the retaining member transferring the respective dose delivery mechanism 54, 354 from the preassembled state into the assembled state. Each adjusting element 18, 318 is rotatable with respect to the retaining member in the preassembled state.

During dose setting in the assembled state, each adjusting element 18, 318 is rotatable with respect to the respective retaining member and, during dose delivery in the assembled state, each adjusting element 18, 318 is rotationally fixed with respect to the respective retaining member. With the dose delivery mechanisms 54, 354, each adjusting element 18, 318 is axially fixed with respect to the respective retaining member in the assembled state.

With the dose delivery mechanisms 54, 354, the first threaded element formed by the piston rod 44 is axially stationary with respect to a third element of the dose delivery mechanism 54, 354 during dose setting in the assembled state. The third element thereby is the housing 32, 332. Furthermore, the second threaded element formed by the nut 38 is axially moved with respect to the third element during dose setting in the assembled state. During adjustment of the piston rod 44 in the preassembled state, the first threaded element formed by the piston rod 44 is axially moved with respect to the third element formed by the housing 32, 332 and the second threaded element formed by the nut 38 is axially stationary with respect to the third element formed by the housing 302, 332.

The dose definition mechanism 115 acting between the snap element 24 and the dose selector 28 of the dose delivery mechanisms 54, 354 is not active in the preassembled state since the adjusting element 318 is rotationally decoupled from the snap element 24 so that the snap element 24 does not rotate upon rotation of the adjusting element 318.

With both dose delivery mechanisms 54, 354, the adjusting element 18, 318 is configured to be rotated in the preassembled state until the bearing 46 touches the distal surface of the plunger 210 after having attached the medicament container 48, 348. A method for adjusting the position of the piston rod 44 in the preassembled state of the dose delivery mechanism 54, 354 can comprise a step of attaching the medicament container 48, 348 to the housing 32, 332 and a step of rotating the adjusting element 18, 318 until the bearing 46 touches the distal surface of the plunger 210. The adjusting element 18, 318 then can further be rotated until the rotation requires a predetermined torque. The dose delivery mechanism 54, 354 can then be transferred from the preassembled state into the assembled state.

The adjusting element 18, 380 can also be rotated until the bearing 46 is located at a distance larger than zero from the distal surface of the plunger 210, thus forming a gap between the distal surface of the plunger 210 and the proximal surface of the bearing 46. The distance can, for example, be measured by measuring the position of the bearing 46 with respect to the plunger 210 through the medicament container 305, which can be made from a transparent material.

Alternatively, the method can also comprise a step of adjusting the position of the piston rod 44 by rotating the adjusting element 18, 318 without the medicament container 48, 348 being attached to the housing 32, 332. The method then can comprise a step of placing the dose delivery mechanism 54, 354 in the preassembled state in an assembly jig and rotating the adjusting element 18, 318 until the proximal surface of the bearing 46 touches a reference surface provided by the assembly jig. The reference surface thereby can be located within the proximal cylindrical portion of the connector 43 of the injection pen 10.

For example, with the injection pen 300, the medicament container 348 can be attached to the housing 332 and the adjusting element 318 then can be rotated until a bearing 46 touches the distal surface of the plunger 210. The adjusting element 318 then can be further rotated until the rotation requires a predetermined torque.

With the dose delivery mechanism 54 of the injection pen 10, the proximal part of the piston rod guide 42 forms a connector 43 that is configured to connect the medicament container 48 axially movable to the housing 32 so that medicament container 48 can perform an axial movement from a receiving position into an operating position after connection to the housing 32. The receiving position thereby is defined by the snap element 234 of the connector 43 engaging with the distal opening 236 of the medicament container holder 50, 52. The operating position is defined by the snap element 234 engaging with the proximal opening 240 of the medicament container holder 50, 52 after having screwed the medicament container holder 50, 52 onto the connector 43.

With the injection pen 10, the dose delivery mechanism 54 can be provided without the medicament container holder 50, 52 being attached to the housing 32 and the position of the piston rod 44 can be adjusted by rotating the adjusting element 18 prior to attaching the medicament container holder 50, 52 to the housing 32. For example, the dose delivery mechanism 54 can be placed in an assembly jig. The adjusting element 18 then can be rotated until the bearing 46 touches a reference surface of the assembly jig and the bearing 46 and the piston rod 44 have reached a predetermined position with respect to the housing 32.

The piston rod 44 thereby is adjusted to a position with respect to the housing 32 that ensures that the bearing 46 gets into contact with the plunger 210 during the movement of the medicament container 48 from the receiving position into the operating position. Furthermore, the position of the piston rod 44 is adjusted to ensure that an amount of the liquid medicament is expelled from the medicament container 48 at the end of the movement into the operating position. For example, the position can be adjusted so that the amount of medicament is expelled only during the last quarter turn of the screwing motion of the medicament container holder 50, 52 onto the proximal part of the piston rod guide 42.

With both the first injection pen 10 and the second injection pen 300, the piston rod 44 can be advanced by a first distance into the proximal direction 1 upon proximal movement of the actuation member 18 by a second distance, wherein the second distance is less than 1.5 times the first distance. With the first injection pen 10, the first distance equals the second distance. Furthermore, the actuation member 18 is traveling the second distance while the piston rod 44 travels the first distance. With the second injection pen 300, the ratio between the second distance and the first distance is given by the ratio of the pitch of the threaded connection 335 between the dosing member 323 and the pitch of the further threaded connection 337 between the driver 336 and the housing 332.

Alternative embodiments of the second injection pen 300 can also comprise a single threaded connection between all members of the second injection pen 300 that rotate during dose delivery and the housing 332. Such alternative embodiments can comprise the dosing member 23 of the first injection pen 10. Like with the first injection pen 10, the dosing member 23 can comprise the driver 36, the dosing element 34 and the snap element 24 shown in FIG. 1.

Additionally or alternatively, the second injection pen 300 can also comprise the drug reconstitution unit 56 of the first injection pen 10. Such a second injection pen 300 then can be configured to receive the double chambered cartridge 48 and to perform reconstitution of a lyophilized drug prior to drug delivery.

Additionally or alternatively, the second injection pen 300 can also be configured to permanently axially lock an actuation member, like the actuation member formed by the adjusting element 318, and/or the dose setting element 22 to the dosing member 323 upon dose delivery. The second injection pen 300 then can comprise the coupling means 100 described in connection with the first injection pen 10.

Generally speaking, the first injection pen 10 and the second injection pen 300 each can comprise a blocking mechanism that is configured to prevent setting and/or delivery of a second dose after having delivered a first dose with the respective injection pen 10, 300. The blocking mechanism can be configured to permanently fix the dose setting element 22 at least rotationally to the housing 32, 332 upon dose delivery, such as upon delivery of a first set dose. Additionally or alternatively, the blocking mechanism can be configured to permanently axially fix the actuation member 18 to a counter member, whereby the actuation member 18 is moved relative to the counter member to initiate delivery of a set dose. With the injection pens 10, 300, the respective counter member is exemplarily formed by the dosing members 23, 323.

The blocking mechanism can comprise a first blocking part that engages a second blocking part to prevent setting and/or delivery of the second dose. With the first and second injection pen 10, 300, the first blocking part is exemplarily formed by the coupling means 100 and the second blocking part is exemplarily formed by the coupling means 102.

The injection pen 10, 300 can comprise a clutch mechanism that rotationally locks the dose setting element 22 to the housing during dose delivery in a closed state of the clutch mechanism. The blocking mechanism can permanently fix the dose setting element 22 at least rotationally to the housing 32, 332 by locking said clutch mechanism in the closed state. The clutch mechanism can, for example, be the clutch mechanism 107.

The clutch mechanism can comprise, for example, a first clutch part, such as the teeth 114 of the connector 26, that engages with a second clutch part, such as the teeth 108 of the dose setting element 22, in the closed state of the clutch mechanism and that disengage from the second clutch part in the opened state of the clutch mechanism. For example, the first and second clutch parts can engage and disengage from each other upon axial relative movement with respect to each other. The clutch mechanism can, for example, be locked in the closed state by axially locking the first clutch part to the second clutch part.

Embodiments of the second injection pen 300 can be configured, like the first injection pen 10, to restrain a user from prematurely activating the second injection pen 300.

Embodiments of the second injection pen 300 can be configured, like the first injection pen 10, to axially lock the actuation member 318 with respect to the housing 332 prior to setting and delivering a first dose. Like the first injection pen 10, the second injection pen 300 can comprise the knob cover 16 and/or the knob key 30.

Like the first injection pen 10, also the second injection pen 300 can be, in an as-delivered condition, preset to an injectable dose that is higher than zero. For example, the dosing member 323 can be preset to a position that corresponds to a set dose higher than zero. The second injection pen 300 can be, like the first injection pen 10, configured to prevent reduction of the preset injectable dose to zero.

With both the first and second injection pen 10, 300, the dosing member 23, 323 is stopped from rotating in a rotational direction at a first angular position during dose setting and stopped from rotating in the rotational direction at a second angular position during dose delivery, whereby the second angular position is spaced in the rotational direction from the first angular position by a distance that corresponds to the preset dose higher than zero. The second angular position can, for example, be taken by the dosing member 23, 323 at the end of dose delivery.

The first and second injection pen 10, 300 can comprise a first stop that limits rotation in the rotational direction at the first angular position during dose setting and/or a second stop that limits rotation of the rotational direction at the second angular position during dose delivery, for example at the end of dose delivery. The first stop can, for example, be the stop 126 shown in FIG. 9 and/or the second stop can, for example, be the end stops 174, 176 shown in FIGS. 13B and 15D.

With alternative embodiments, a stop that prevents the rotation of the dosing members 23, 323 at the end of dose delivery can also be provided at the respective dose selector 28. The stop can, for example, interact with a corresponding stop provided at the dosing member 23, 323, such as the hard stop 124. Such a stop is disclosed in document WO 2020015980 A1, the disclosure of which is incorporated into the present disclosure in its entirety by reference, including the configuration of the stop, which is referred to as zero dose hard stop in document WO 2020015980 A1.

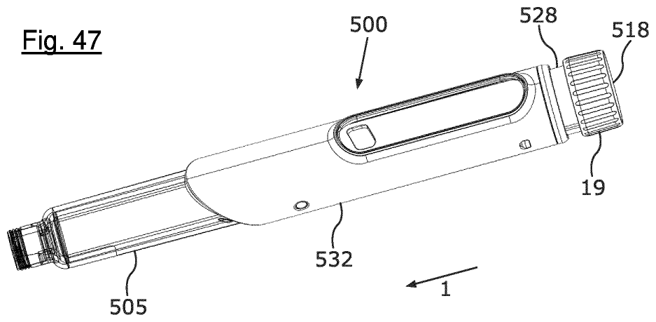

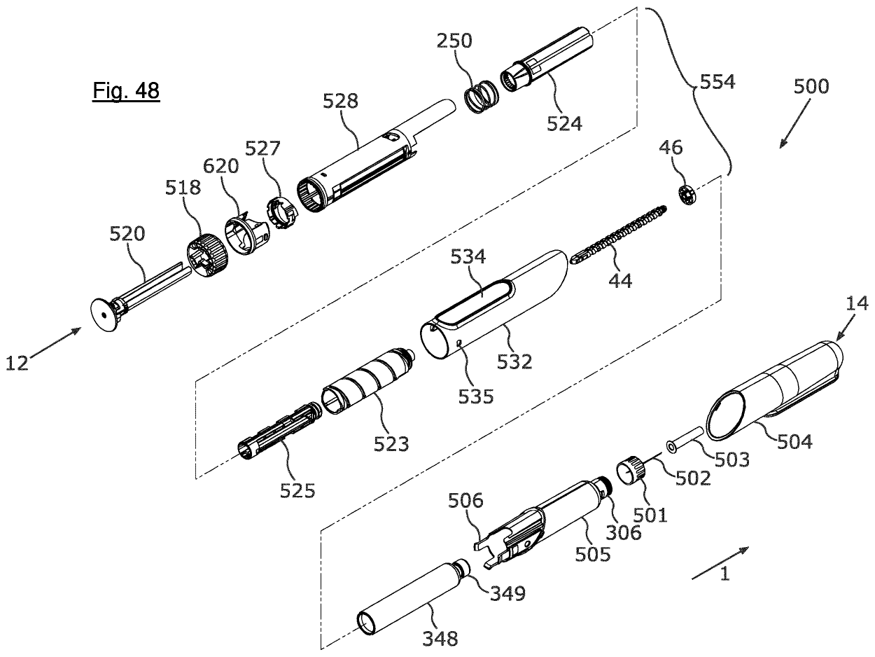

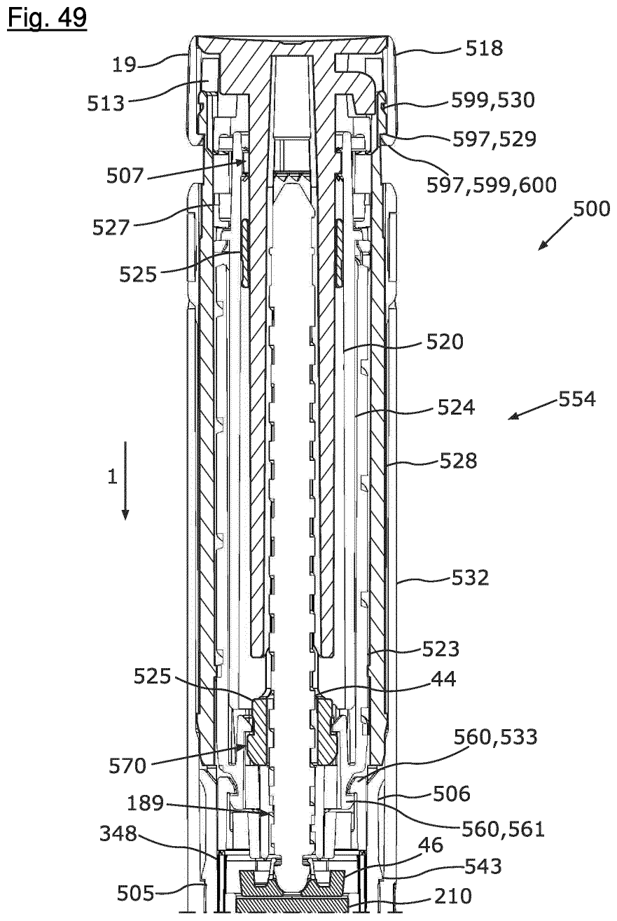

FIG. 47 shows a perspective view of a third injection pen 500 according to the present disclosure, FIG. 48 shows an exploded view of the third injection pen 500 and FIG. 49 shows a longitudinal cut through a dose delivery mechanism 554 of the third injection pen 500 in an assembled state during dose setting. As far as no differences are disclosed in the description or the Figures, the third injection pen 500 is configured as it is disclosed for the second injection pen 300 and vice versa.

The dose delivery mechanism 554 comprises a housing 532 that is configured to connect to a medicament container holder 505 via a non-releasable form-fit connection. The connection comprises a connector 506 located at the distal end of the medicament container holder 505. The connector 506 is configured to engage with a corresponding connector 543 located at the proximal end of the housing 532, see FIG. 49. The connection is configured as a non-releasable snap fit connection.

The medicament container holder 505 is configured to receive the medicament container 348 already described in connection with the second injection pen 300. At a proximal end, the medicament container holder 505 comprises a needle connector 306 that is configured to receive a double ended needle assembly 501 having a double ended cannula 502. The needle connector 300 connects to the needle assembly 501 via a threaded connection. Alternatively, the connection could also be configured as a Luer lock, a snap fit connection or the like. Upon mounting the needle assembly 501 onto the medicament container holder 505, a distal end of the cannula 502 pierces the septum at the proximal needle end 349 of the medicament container 348. The proximal end of the cannula 502 is covered by a needle cap 503 that is removed before use of the injection pen 500. During storage of the injection pen 500, a cap 504 covers the medicament container holder 505.

FIG. 49 depict the dose delivery mechanism 554 with all components of the dose delivery mechanism 554 except for the piston rod 44 and components axially fixed to the piston rod 44, such as the bearing 46, being axially located in their zero dose position with respect to the housing 532.

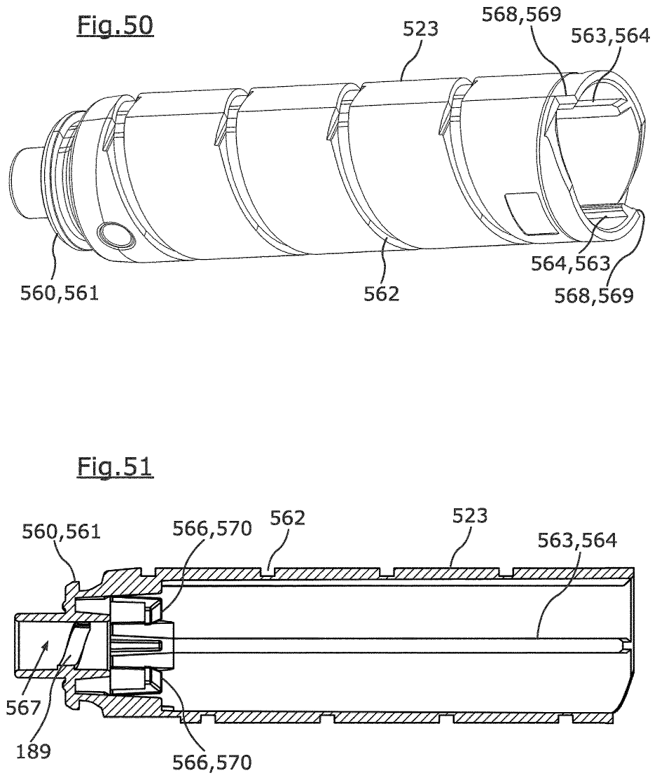

The dose delivery mechanism 554 comprises a dosing member 523 that is axially fixed and rotationally movable with respect to the housing 532 by a rotatable fixation 560. FIG. 50 depicts a perspective distal view of the dosing member 523 and FIG. 51 depicts a longitudinal cut through the dosing member 523. The rotatable fixation 560 comprises an annular rim 561 located at the proximal end of the dosing member 523 and corresponding holding lugs 533 at the proximal end of the housing 532. The holding lugs 533 snap behind the annular rim 561 and thus axially fix the dosing member 523 to the housing 532.

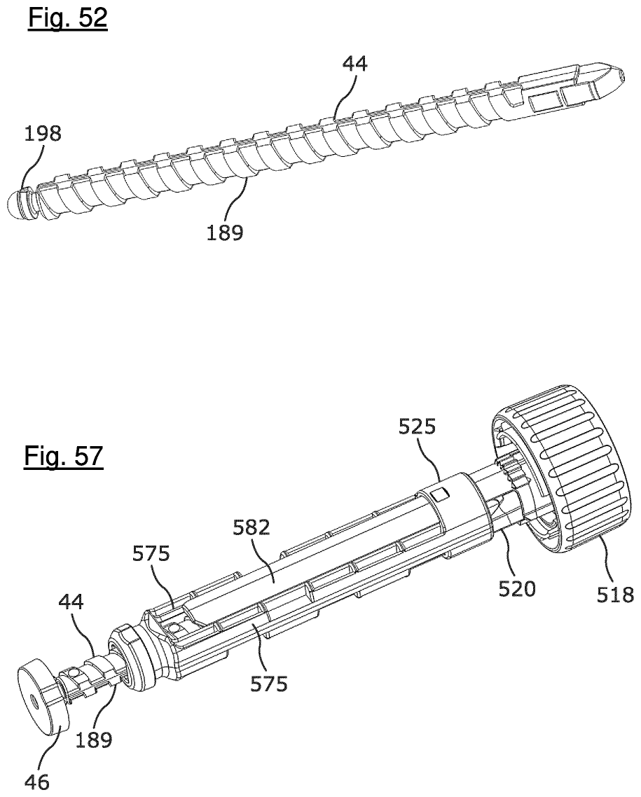

The dose delivery mechanism 554 further comprises a piston rod 44, which is shown in FIG. 52 in a perspective view. The piston rod 44 is received in an opening 567 at the proximal end of the dosing member 523. Thereby, the piston rod 44 is connected to the dosing member 523 via a threaded connection 189. The threaded connection 189 comprises an inner thread within the opening 567 of the dosing member 523 and an outer thread provided at the outer circumference of the piston rod 44. The piston rod 44 forms a first threaded element of the threaded connection 189 and the dosing member 523 forms a second threaded element of the threaded connection 189. At its proximal end, the piston rod 44 comprises coupling means 198 that connect a bearing 46 axially fixed and rotationally movable to the piston rod 44.

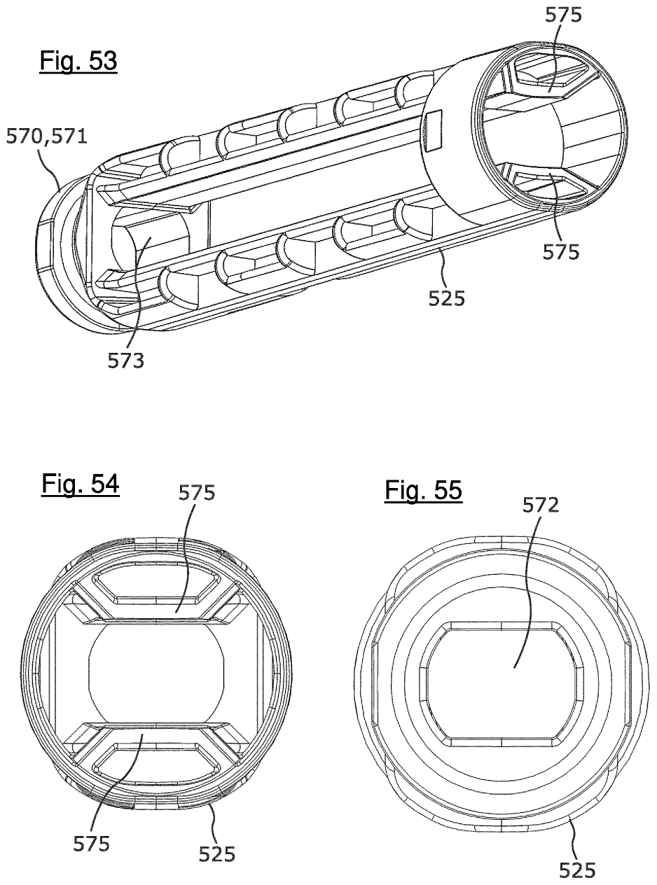

FIG. 53 depicts a perspective distal view of an extension 525 of the dose delivery mechanism 554, FIG. 54 depicts a distal view of the extension 525 and FIG. 55 depicts a proximal view of the extension 525. The extension 525 is received within the dosing member 523. It is held axially fixed and rotationally movable within the dosing member 523 by a rotatable fixation 570. The rotatable fixation 570 exemplarily comprises an annular ridge 571 located at the proximal end of the extension 525 and corresponding lugs 566 provided at the proximal end of the dosing member 523. The lugs 566 snap behind the annular ridge 571 from the proximal side of the extension 525 and thereby axially fix the extension 525 to the dosing member 523.

The piston rod 44 is axially movable and rotationally fixed with respect to the extension 525. At its proximal end, the extension comprises a non-circular opening 573 that is adapted to a corresponding non-circular outer shape of the piston rod 44. The piston rod 44 is received within the opening 573, thereby rotationally locking the piston rod 44 to the extension 525, while allowing relative axial movement between the piston rod 44 and the extension 525.

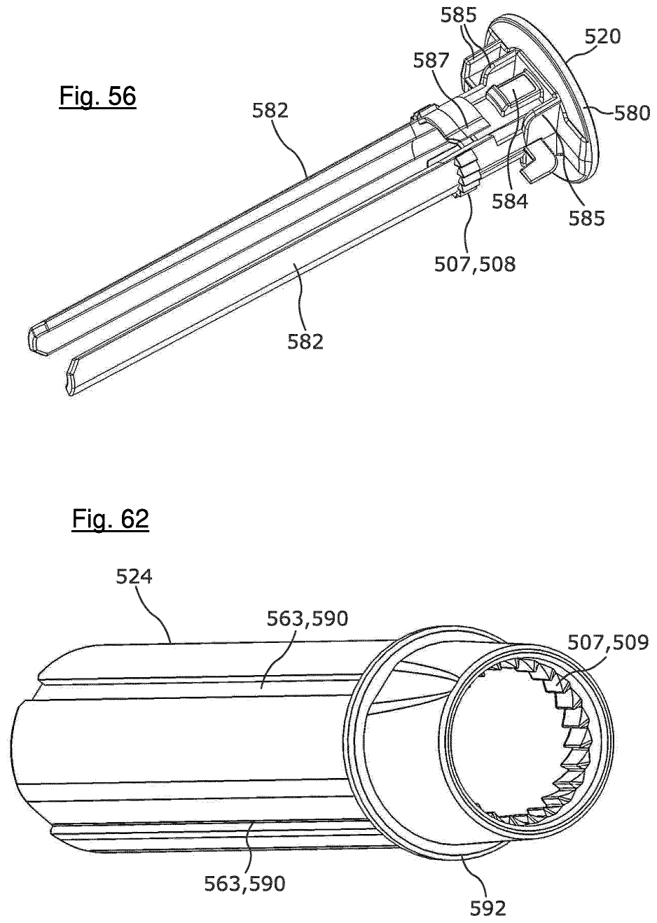

FIG. 56 depicts a perspective view of a coupling element 520 of the dose delivery mechanism 554. The coupling element 520 comprises an end plate 580 located at its distal end and two bars 582 that extend axially and parallel to each other in the proximal direction.

The coupling element 520 is rotationally fixed and axially movable with respect to the piston rod 44. It thereby is coupled to the piston rod 44 via the extension 525. As can be seen from FIG. 57, the bars 582 of the coupling element 520 are received in between two ridges 575 of the extension 525 that radially extend from an inside surface of the extension 525. The ridges 575 thereby run parallel to each other along the axial direction. The bars 582 and the ridges 575 provide an axially movable connection between the coupling element 520 and the extension 525 that rotationally fixes the coupling element 520 to the extension 525.

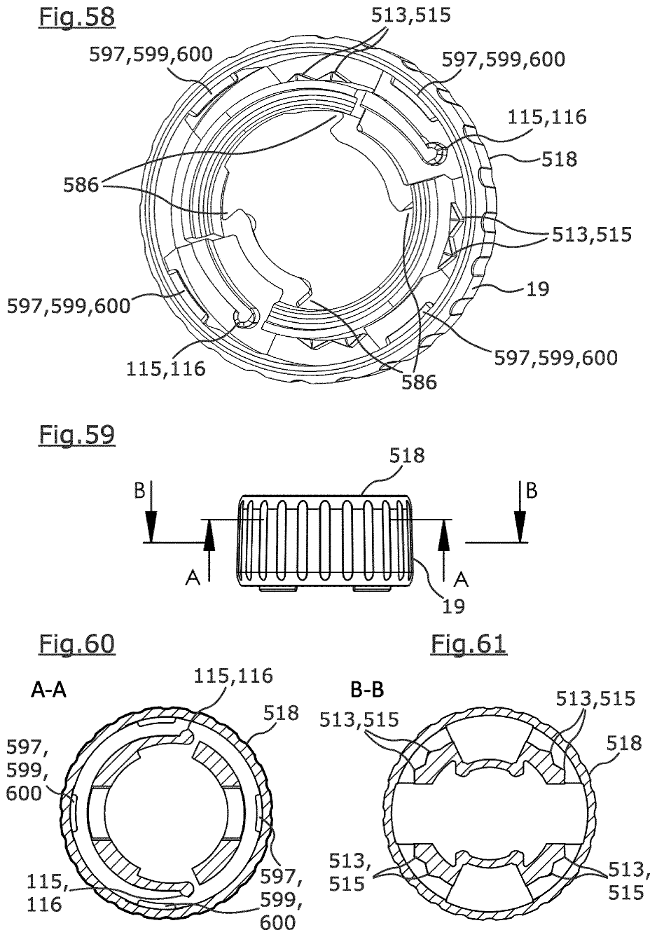

As further can be seen from FIG. 57, an adjusting element 518 is axially and rotationally fixed to the distal end of the coupling element 520. FIG. 58 depicts a proximal perspective view of the adjusting element 518, FIG. 59 depicts a side view of the adjusting element 518, FIG. 60 depicts a radial cut through the adjusting element 518 along the line A-A in FIG. 59 and FIG. 61 depicts a radial cut through the adjusting element 518 along the line B-B in FIG. 59.

The adjusting element 518 is axially and rotationally fixed to the coupling element 520. The adjusting element 518 and the coupling element 520 thus form a single member of the dose delivery mechanism 554. As can be seen from FIG. 56, the coupling element 520 comprises an axial fixation element 584 that is part of an axial fixation acting between the adjusting element 518 and the coupling element 520 and that engages with a corresponding axial fixation element provided at adjusting element 518. The axial fixation element 584 of the coupling element 520 thereby is configured as a snap hook and the corresponding axial fixation element of the adjusting element 518 is configured as a circumferential edge that engages with the snap hook. The coupling element 520 further comprises at least one rotational fixation element 585, for example several rotational fixation elements 585. The rotational fixation elements 585 engage with corresponding rotational fixation elements provided at the adjusting element 518 and thereby rotationally lock the adjusting element 518 to the coupling element 520. The rotational fixation elements 585 of the coupling element 520 are configured is longitudinally ridges that run parallel to the axial direction. The ridges are received in between corresponding longitudinal recesses 586 provided within the adjusting element 518. With other embodiments of the dose delivery mechanism 554, the adjusting element 518 and the coupling element 520 can also be configured as a one-pieced single member.

FIG. 62 depicts a perspective view of a coupling member 524 of the dose delivery mechanism 554. The coupling member 524 is configured as a hollow member. As can be seen from FIG. 49, the coupling member 524 is located in between the dosing member 523 and the extension 525. Thereby, the coupling member 524 is placed within the dosing member 523 and receives the extension 525 in an inner cavity. The coupling member 524 is rotationally fixed to the dosing member 523 via an axially movable rotation fixation 563. The rotation fixation 563 comprises longitudinal recesses 590 provided on an outer surface of the coupling member 524 that engage corresponding longitudinal ridges 564 provided on an inside surface of the dosing member 523, see FIGS. 50 and 51.

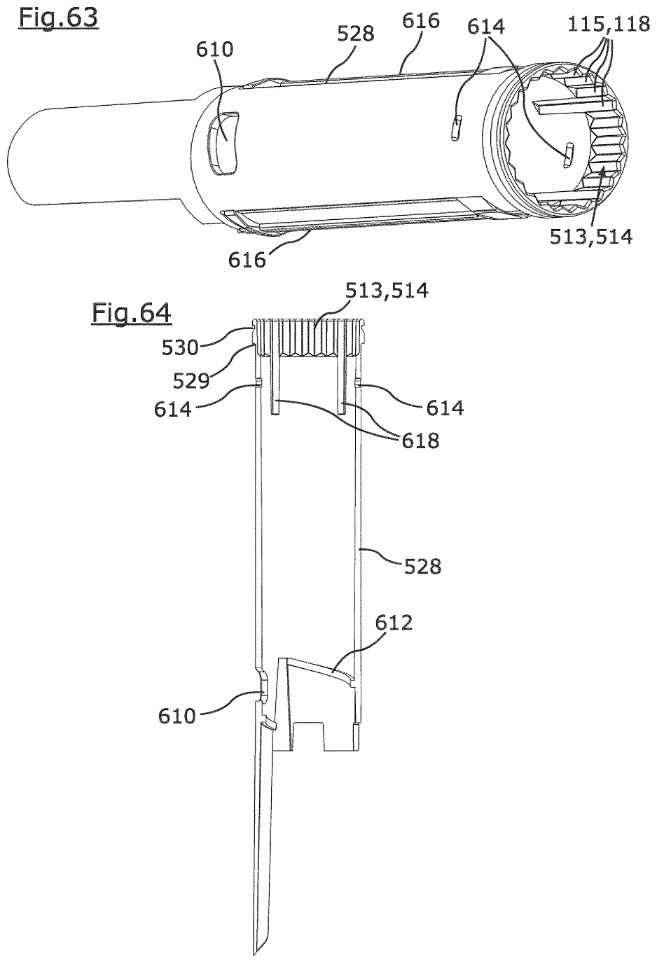

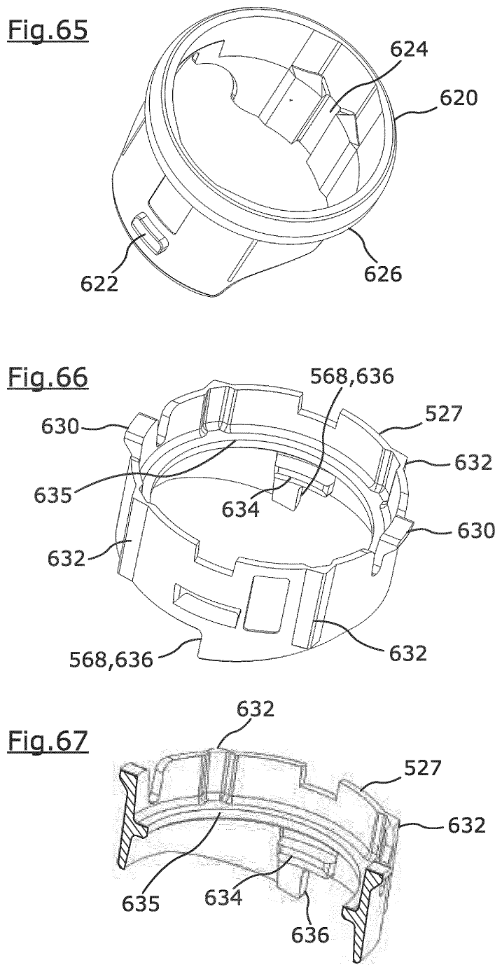

FIG. 63 depicts a perspective distal view of a sleeve 528 of the dose delivery mechanism 554 and FIG. 64 depicts a longitudinal cut through the sleeve 528. The sleeve 528 is configured as a hollow member. As can be seen from FIG. 49, the sleeve 528 is located in between the housing 532 and the dosing member 523.

The sleeve 528 is threadedly connected to and threadedly engaged with the dosing member 523. A threaded connection between the sleeve 528 and the dosing member 523 comprises an inner thread 612 provided on an inside surface of the sleeve 528 that engages an outer thread 562 provided on an outer surface of the dosing member 523.

Furthermore, the sleeve 528 is rotationally fixed and axially movable connected to the housing 532. A connection between the sleeve 528 and the housing 532 thereby comprises a connector 620. The connector 620 is located at the distal end of the housing 532. It is axially and rotationally fixed with respect to the housing 532. With other embodiments, the connector 620 can also be formed integrally with the housing 532. The connector 620 comprises a pair of radial lugs 622 that are provided at an outer surface of the connector 620. The radial lugs 622 engage with corresponding openings 535 accessible at an inside surface of the housing 532. The connector 620 further comprises an outer annular rim 626 provided at a distal end of the connector 620. The annular rim 626 rests against the distal surface of the housing 532, thereby preventing the connector 620 from moving in the proximal direction.

On an inside surface of the connector 620, longitudinal recesses 624 are provided that engage with corresponding longitudinal ridges 616 on an outer surface of the sleeve 528. This provides a rotationally fixed and axially movable connection between the housing 532 and the sleeve 528.

The dose sleeve 523 is configured as a dose indication member and comprises markings on its outer surface that serve to indicate a set dose. A window 610 is formed within the sleeve 528, through which the dose sleeve 523 is visible. The window 610 of the sleeve 528 is aligned with a housing window 534 provided within the housing 532, so that the dose sleeve 523 is visible from the outside of the housing 532. A set dose is then indicated by the marking that is visible through the windows 534, 610.

The coupling member 524 is axially fixed and rotationally movable with respect to the sleeve 528. A connection between the coupling member 524 and the sleeve 528 comprises a connector 527.

FIG. 66 depicts a perspective view of the connector 527 and FIG. 67 depicts a perspective view of a longitudinal cut through the connector 527. The connector 527 is axially and rotationally fixed with respect to the sleeve 528. It comprises longitudinal ridges 632 on its outer surface that engage with corresponding recesses 618 (see FIG. 64) provided on the inside surface of the sleeve 528. Furthermore, the connector 527 comprises radially extending lugs 630 that engage with openings 614 accessible on the inside surface of the sleeve 528. This engagement prevents the connector 527 from being removed from the sleeve 528.

On an inside surface of the connector 527, a distal blocking element 635 and proximal blocking elements 654 are formed. The blocking elements 635, 654 provide an axially fixed and rotationally movable connection to the coupling member 524. Thereby, an annular rim 592 that is provided on the outer surface of the coupling member 524 and that extends in the radial direction (see FIG. 62), is received in between the blocking elements 635, 654.

Furthermore, a radial stop 568 is formed between the sleeve 528 and the dosing member 523. This radial stop 568 is configured to stop relative rotation between the dosing member 523 and the sleeve 528 and thus also between the dosing member 523 and the housing 532 at the end of dose delivery. The radial stop 568 comprises at least one stop surface 569 provided at the dosing member 523 and a corresponding stop surface 636 provided at the connector 527. The stop surfaces 569, 636 are orientated parallel to each other and configured to engage with each other at the end of dose delivery. The stop surfaces 569, 636 form an angle with a radial plane orientated perpendicular to the longitudinal axis of the dose de-livery mechanism 554. With the third injection pen 500, the stop surfaces 569, 636 are orientated parallel to the longitudinal axis. While the stop surface 636 is provided at the connector 527, the stop surface 636 can also be provided directly at the sleeve 528 with other embodiments.

With the third injection pen 500, the adjusting element 518 forms a dose setting member of the dose delivery mechanism 554. To set a dose to be delivered, a user rotates the adjusting element 518 with respect to the housing 532 in the assembled state.

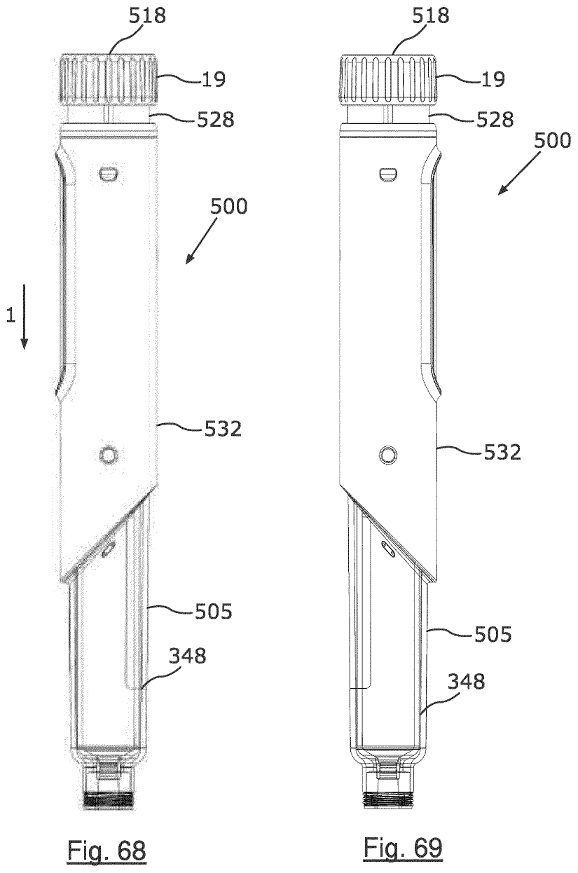

FIG. 68 depicts a side view of the third injection pen 500 in an assembled state during dose setting, when no dose is set. FIG. 69 depicts a side view of the third injection pen 500 in a preassembled state. In the preassembled state, the adjusting element 518 is located in a preassembled position with respect to the housing 532 and in the assembled state during dose setting and with no dose being set, the adjusting element 518 is in an assembled position with respect to the housing 532. Thereby, the assembled position is located more proximally than the preassembled position.

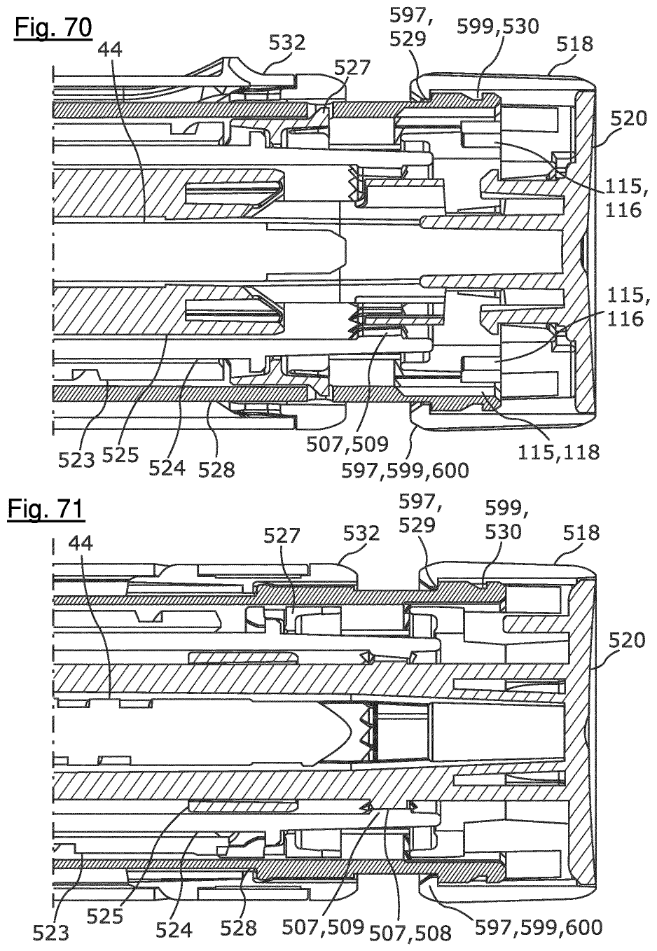

FIG. 70 shows a detailed view of a longitudinal cut through the distal end of the third injection pen 500 in the assembled state during dose setting and FIG. 71 shows a detailed view of a further longitudinal cut through the distal end of the third injection pen 500 in the assembled state during dose setting. Thereby, a longitudinal cut plane of the view shown in FIG. 71 is orientated perpendicular to a longitudinal cut plane of the view shown in FIG. 70.

During dose setting, the adjusting element 518 is rotationally fixed with respect to the coupling member 524 by a clutch mechanism 507. Rotation of the adjusting element 518 then causes rotation of the piston rod 44 due to the rotational fixation via the extension 525 and the coupling element 520 and simultaneous rotation of the dosing member 523 due to the rotational fixation via the coupling element 520, the clutch mechanism 507 and the coupling member 524. Since both the piston rod 44 and the dosing member 523 rotate with respect to the housing 532 at the same speed during dose setting, the piston rod 44 does not change its axial position with respect to the housing 532 despite the threaded connection 189 between the piston rod 44 and the dosing member 523.

Rotation of the dosing member 523 with respect to the sleeve 528 during dose setting causes the sleeve 528 to move axially in the distal direction with respect to the housing 532 due to the threaded connection 562, 612. This also causes distal movement of the adjusting element 518 and the coupling element 520. Furthermore, the coupling member 524 is also moved distally due to the axially fixed and rotationally movable connection to the sleeve 528 via the connector 527.

As can be seen from FIGS. 70 and 71, the adjusting element 518 is coupled to the sleeve 528 by a latching mechanism 597 that prevents distal movement of the adjusting element 518 and the coupling element 520 with respect to the sleeve 528. As can be seen from FIGS. 58 to 61, 63 and 64, the latching mechanism 597 comprises a latch part 600 located at the proximal end of the adjusting element 518 that engages with a latch counterpart 529 of the sleeve 528. The latch counterpart 529 of the sleeve 528 is configured as an annular edge located at the outside surface of the sleeve 528. The latch part 600 of the adjusting element 518 is configured as corresponding radial lugs provided on an inner surface of the adjusting element 518. In the assembled position of the adjusting element 518, the radial lugs 600 engage with the annular edge 529, thus preventing further distal movement of the adjusting element 518 into the preassembled.

The adjusting element 518 and the coupling element 520 are biased with respect to the sleeve 528 in the distal direction by a biasing member 250, which is configured as a compression spring and which is shown in FIG. 48 and which is not visible in FIGS. 70 and 71.

During the rotation of the dosing member 523 and the axial movement of the sleeve 528 with respect to the housing 532 during dose setting, the window 610 of the sleeve 528 axially moves along the dosing member 523. Thereby, a respective marking on the dose sleeve 523 that is visible through the window 610 indicates a dose that is currently set.

To deliver a set dose, a user of the third injection pen 500 pushes the adjusting element 518 and the coupling element 520 in the proximal direction 1 against the force of the biasing member 250.

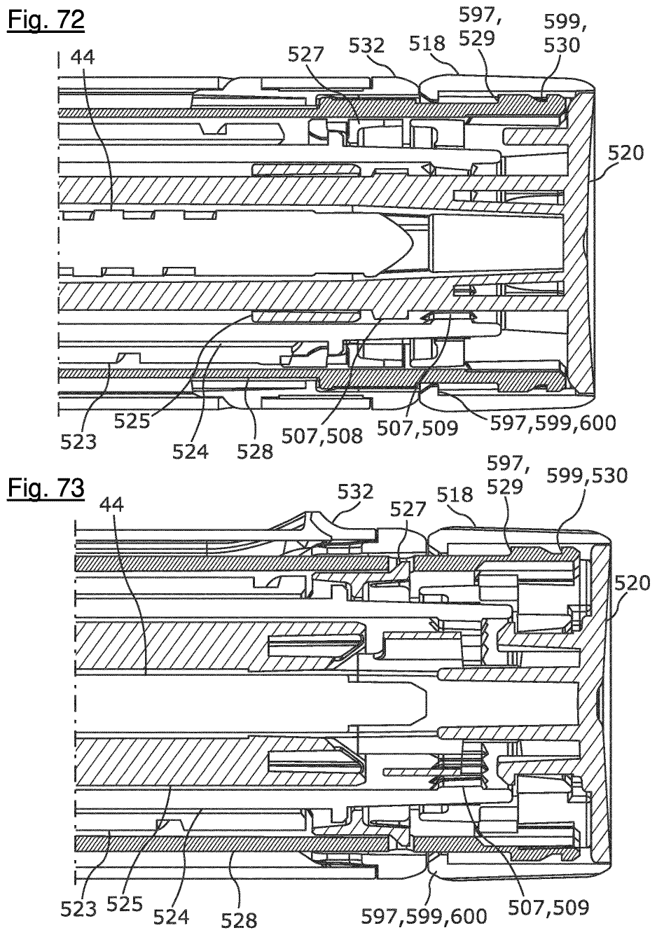

FIG. 72 shows a detailed view of a longitudinal cut through the distal end of the third injection pen 500 in the assembled state during dose delivery and FIG. 73 shows a detailed view of a further longitudinal cut through the distal end of the third injection pen 500 in the assembled state during dose delivery, whereby a longitudinal cut plane is orientated perpendicular to a longitudinal cut plane of the view in FIG. 73. FIGS. 72, 73 thereby show the dose delivery mechanism 554 of the third injection pen 500 at the end of dose delivery, when the set dose has been fully expelled and the user still presses the adjusting element 518 and the coupling element 520 in the proximal direction.

During dose delivery, the adjusting element 518 and the coupling element 520 are rotationally locked to the housing 532 via the sleeve 528. This is because proximal movement of the coupling element 520 and the adjusting element 518 with respect to the sleeve 528 at the beginning of dose delivery closes a clutch mechanism 513 between the adjusting element 518 and the sleeve 528. The clutch mechanism 513 comprises teeth 515 formed at the adjusting element 518 and corresponding teeth 514 formed at the distal end of the sleeve 528. The clutch mechanism 513 also rotationally locks the piston rod 44 to the housing 532 during dose delivery via the extension 525, the coupling element 520, the adjusting element 518 and the sleeve 528.

Proximal movement of the coupling element 520 with respect to the coupling member 524 at the beginning of dose delivery causes the clutch mechanism 507 between the coupling element 520 and the coupling member 524 to open so that the coupling member 524 becomes rotatable with respect to the coupling element 520. After disengagement of the clutch mechanism 507, further proximal movement of the coupling element 520 pushes the sleeve 528 in the proximal direction 1. The proximal movement of the sleeve 528 rotates the dosing member 523 via the threaded connection 612 between the sleeve 528 and the dosing member 523. Since the piston rod 44 is rotationally locked to the housing 332 during dose delivery, rotation of the dosing member 523 causes proximal movement of the piston rod 44, which proximal movement is driven via the threaded connection 189.

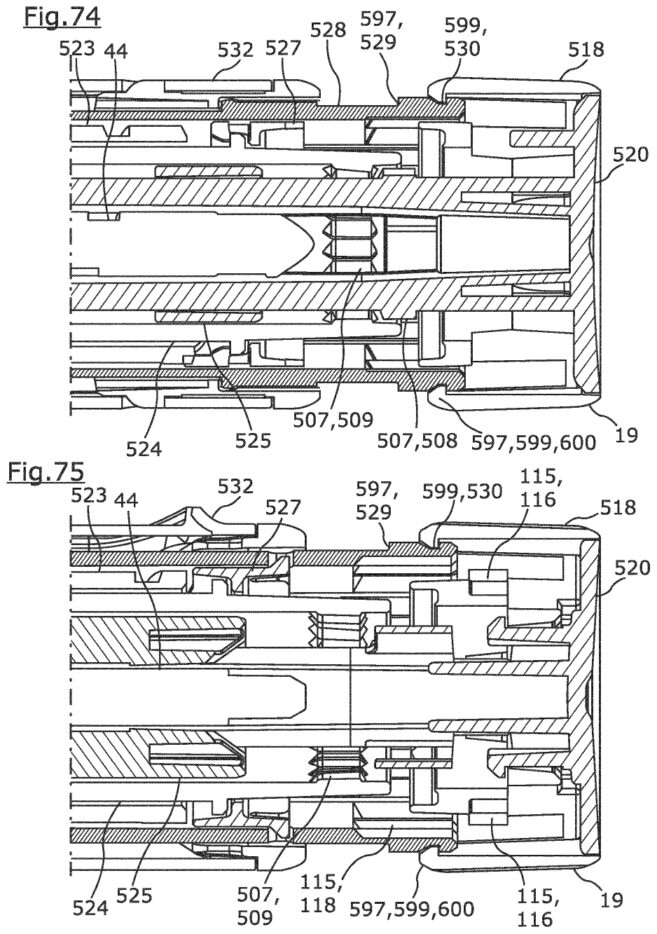

FIG. 74 shows a detailed view of a longitudinal cut through the distal end of the third injection pen 500 in a preassembled state of the dose delivery mechanism 554 and FIG. 75 shows a detailed view of a further longitudinal cut through the distal end of the third injection pen 500 in the preassembled state, whereby a longitudinal cut plane is orientated perpendicular to a longitudinal cut plane of the view in FIG. 74.

In the preassembled state, the adjusting element 518 and the coupling element 520 are located in the adjusting position with respect to the housing 532 and the sleeve 528. In the adjusting position, the adjusting element 518 and the coupling element 520 are shifted in the distal direction with respect to their respective assembled positions in the assembled state.

The dose delivery mechanism 554 comprises a further latching mechanism 599 that prevents detachment of the adjusting element 518 and the coupling element 520 in the preassembled state. The further latching mechanism 599 comprises the latch part 600 of the adjusting element 518 and a further latch counterpart 530 located at the distal end of the sleeve 528. The further latch counterpart 530 thereby is located distally from the latch counterpart 529.

The further latch counterpart 530 is configured as an angular recess that receives the radial lugs of the latch part 600 of the adjusting element 518. The latch parts 600 formed by the radial lugs thereby are releasably engaged with the further latch counterpart 530 and allow proximal movement of the adjusting element 518 while blocking distal movement.

In the preassembled state, the clutch mechanism 507 between the coupling element 520 and the coupling member 524 is opened so that the adjusting element 518 is rotationally decoupled from the dosing member 523. At the same time, the adjusting element 518 is rotationally coupled and rotationally fixed with respect to the piston rod 44 by the coupling element 520 and the extension 525. Rotation of the adjusting element 518 with respect to the housing 532 thereby causes the piston rod 44 to rotate with respect to the housing 532 and the dosing member 523. Due to the threaded connection 189 between the dosing member 523 and the piston rod 44, the piston rod 44 moves axially with respect to the housing 332 upon rotation of the adjusting element 518.

With the clutch mechanism 507, the coupling element 520 forms a first clutch member of the clutch mechanism 507 and the coupling member 524 forms a second clutch member of the clutch mechanism 507. The dosing member 523 of the dose delivery mechanism 554 forms a further member of the dose delivery mechanisms 54, 354 to which the adjusting element 518 is rotationally coupled during dose setting in the assembled state and from which the adjusting element 518 is rotationally decoupled during dose delivery in the assembled state.

The coupling element 520 comprises a first clutch part 508 of the clutch mechanism 507. The first clutch part 508 is configured as radial teeth that are provided on an outer surface of the coupling element 520. The coupling member 524 comprises a second clutch part 509 of the clutch mechanism 507. The second clutch part 509 is configured as radial teeth that are located on the inside surface of the coupling member 524. In the closed state of the clutch mechanism 507, the first clutch part 508 is engaged with the second clutch part 509, as it is shown in FIG. 71.

During the adjustment of the piston rod in the preassembled state, the clutch mechanism 507 is in an opened state. Thereby, the first clutch part 508 and the second clutch part 509 are brought out of engagement by locating them at an axial distance from each other. The first clutch part 508 thereby is shifted in a distal direction from the second clutch part 509, the distal direction being opposite the proximal direction 1, see FIG. 74. During dose delivery in the assembled state, the clutch mechanism 507 is also in an opened state.

Thereby, the first clutch part 508 and the second clutch part 509 are also brought out of engagement by locating them at an axial distance from each other, whereby the second clutch part 509 is shifted in the proximal direction 1 from the first clutch part 508, see FIG. 72.

Furthermore, the adjusting element 518 forms a first clutch member of the clutch mechanism 513 and the sleeve 528 forms a second clutch member of the clutch mechanism 513. The housing 532 of the dose delivery mechanism 554 forms an additional member of the dose delivery mechanism 554 to which the adjusting element 518 is rotationally coupled during dose setting in the assembled state and from which the adjusting element 518 is rotationally decoupled during dose delivery in the assembled state.

The sleeve 528 forms a retaining member for the adjusting element 518. In the preassembled state shown in FIGS. 74 and 75, the adjusting element 518 is in a first axial position with respect to the retaining member 528. Furthermore, in the assembled state shown in FIGS. 70 and 71, the adjusting element 518 is in a second axial position with respect to the retaining member 528. The first axial position thereby is a more distal axial position than the second axial position. In the assembled state, the adjusting element 518 is in the second axial position with respect to the retaining member 528 at least during dose setting. Furthermore, it is biased into the second axial position by the spring acting between the sleeve 528 and the adjusting element 518.

The dose delivery mechanism 554 comprises a dose definition mechanism 115 that defines the doses settable by a user. Engagement features 116 of the dose definition mechanism 115 are provided at the adjusting element 518, see FIG. 58. The engagement features 116 are configured as radially extending lugs that are flexible in the radial direction. The engagement features 116 engage with corresponding dose stops 118 that are provided at a distal end of the sleeve 528, see FIGS. 63, 64. The dose stops 118 are configured as longitudinally recesses formed at an inner surface of the sleeve 528. During dose setting, the adjusting element 518 and the coupling element 520 are configured to perform more than one full revolution about the longitudinal axis of the dose delivery mechanism 554.

During adjustment of the piston rod 44 in the preassembled state, the dose definition mechanism 115 of the dose delivery mechanism 554 is not active. This is because the engagement features 116 are axially shifted with respect to the dose stops 118 to bring the engagement features 116 and the dose stops 118 out of mutual engagement, see FIG. 75.

With the dose delivery mechanism 554, the housing 532 forms a third element and the first threaded element formed by the piston rod 44 is rotated with respect to that third element during adjustment of the piston rod in the preassembled state, while the second threaded element formed by the dosing member 523 is rotationally fixed with respect to the third element. During dose delivery in the assembled state, the first threaded element formed by the piston rod 44 is rotationally fixed with respect to the third element formed by the housing 532 and the second threaded element formed by the dosing member 523 is rotated with respect to the third element.

With the third injection pen 500, the piston rod 44 can be configured to advance by a first distance into the proximal direction 1 upon proximal movement of the actuation member 520 by a second distance, wherein the second distance is less than 1.5 times the first distance. With the third injection pen 500, the ratio between the second distance and the first distance is given by the ratio of the pitches of the threaded connection 189 between the piston rod 44 and the dosing member 523 and of the further threaded connection 562 between the sleeve 528 and the dosing member 523.

Additionally or alternatively, the third injection pen 500 can also comprise the drug reconstitution unit 56 of the first injection pen 10. Such a third injection pen 500 then can be configured to receive the double chambered cartridge 48 and to perform reconstitution of a lyophilized drug prior to drug delivery.

Embodiments of the third injection pen 500 can be configured, like the first injection pen 10, to restrain a user from prematurely activating the third injection pen 500. Embodiments of the third injection pen 500 can be configured, like the first injection pen 10, to axially lock the actuation member 520 with respect to the housing 532 prior to setting and delivering a first dose. Like the first injection pen 10, the third injection pen 500 can comprise the knob cover 16 and/or the knob key 30.

FIG. 76 shows a perspective view of a fourth injection pen 700 according to the present disclosure. The fourth injection pen 700 is a variant of the third injection pen 500. As long as no differences are disclosed in the description or the Figures, the fourth injection pen 700 is configured as it is disclosed for the third injection pen 500. In the following, components of the fourth injection pen 700 that perform the same functions as corresponding components of the third injection pen 500 are labeled with the same reference signs. These components can, however, differentiate among the third injection pen 500 and the fourth injection pen 700 in shape and/or appearance.

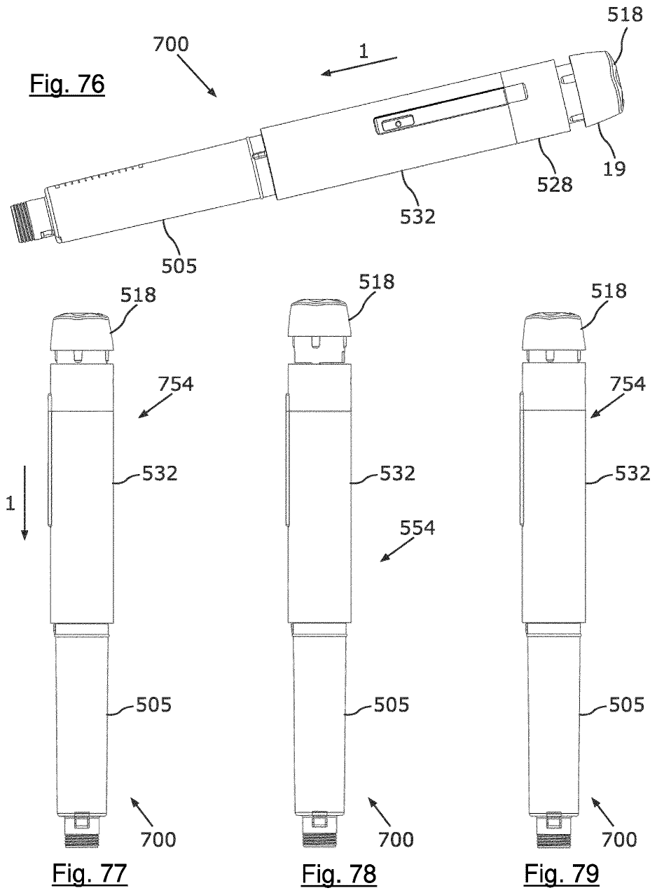

FIG. 77 shows a side view of the fourth injection pen 700 in an assembled state during dose setting. Thereby, no dose is set and an adjusting element 518 of a dose delivery mechanism 754 of the fourth injection pen 700 is positioned in an assembled position with respect to a housing 532 of the dose delivery mechanism 754.

FIG. 78 shows a side view of the fourth injection pen 700 in a preassembled state with the adjusting element 518 being in a preassembled position with respect to the housing 532. In the preassembled position, the adjusting element 518 is shifted in a distal direction from its assembled position, whereby the distal direction is orientated perpendicular to a proximal direction 1.

FIG. 79 shows a side view of the fourth injection pen in the preassembled state with the adjusting element 518 in an adjusting position. In the adjusting position, the adjusting element 518 is shifted in the proximal direction compared to the preassembled position.

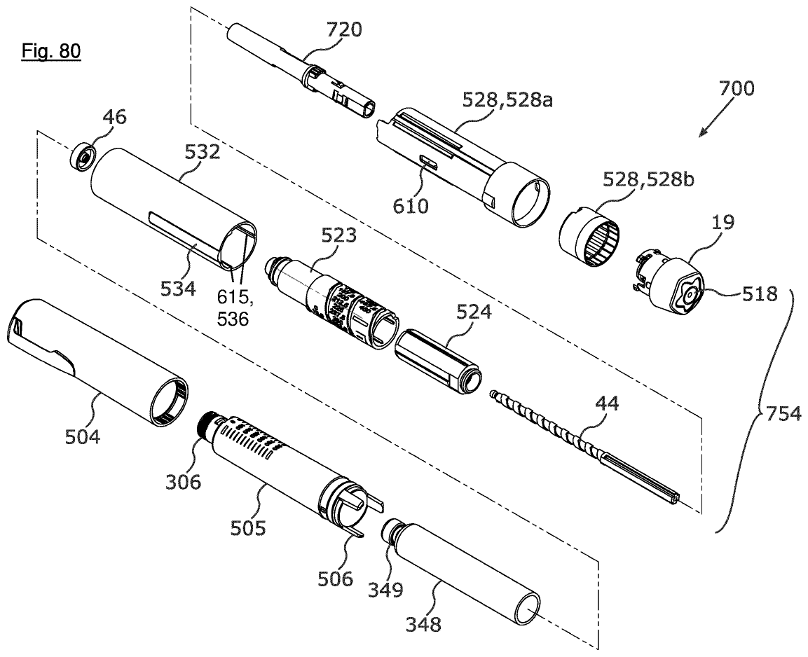

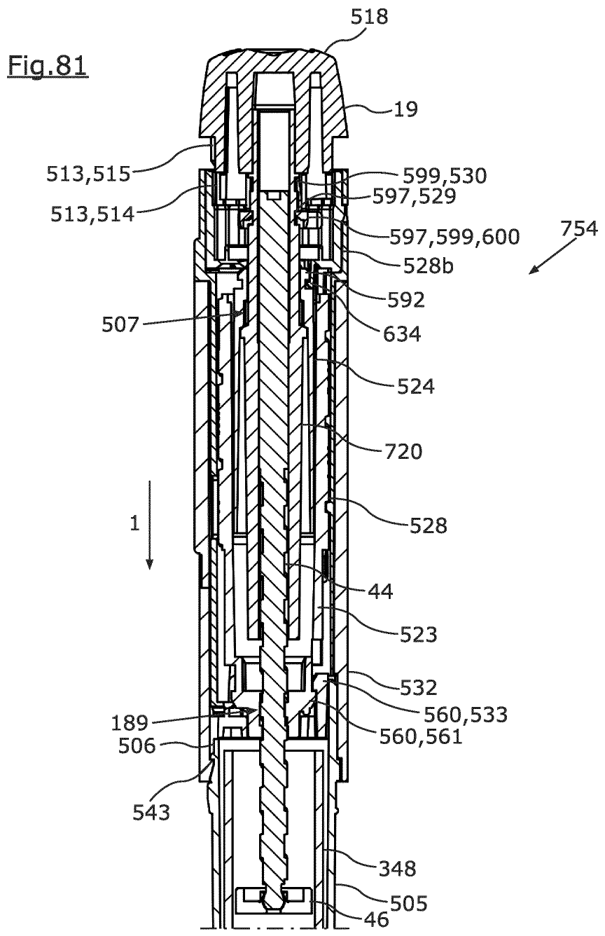

FIG. 80 shows an exploded view of the fourth injection pen 700 and FIG. 81 shows a longitudinal cut through the dose delivery mechanism 754 of the fourth injection pen 700 in the assembled state during dose setting with no dose set. All components of the dose delivery mechanism 754 except for the piston rod 44 and components axially fixed to the piston rod 44, such as the bearing 46, thereby are axially located in their zero dose position with respect to the housing 532.

Like the dose delivery mechanism 554 of the third injection pen 500, the dose de-livery mechanism 754 of the fourth injection pen 700 comprises a coupling element 720 that is rotationally fixed and axially movable with respect to a piston rod 44. Unlike the dose de-livery mechanism 554, the dose delivery mechanism 754 does not feature the extension 525. Instead, the coupling element 720 directly engages with the piston rod 44 to rotationally fix the coupling element 720 to the piston rod 44 and to allow axial movement between the coupling element 720 and the piston rod 44.

The dose delivery mechanism 754 furthermore comprises a biasing element in the form of a spring, which is not shown in FIGS. 80 and 81. The biasing element biases the adjusting element 518 in the distal direction both in the preassembled state and in the assembled state of the dose delivery mechanism 754.

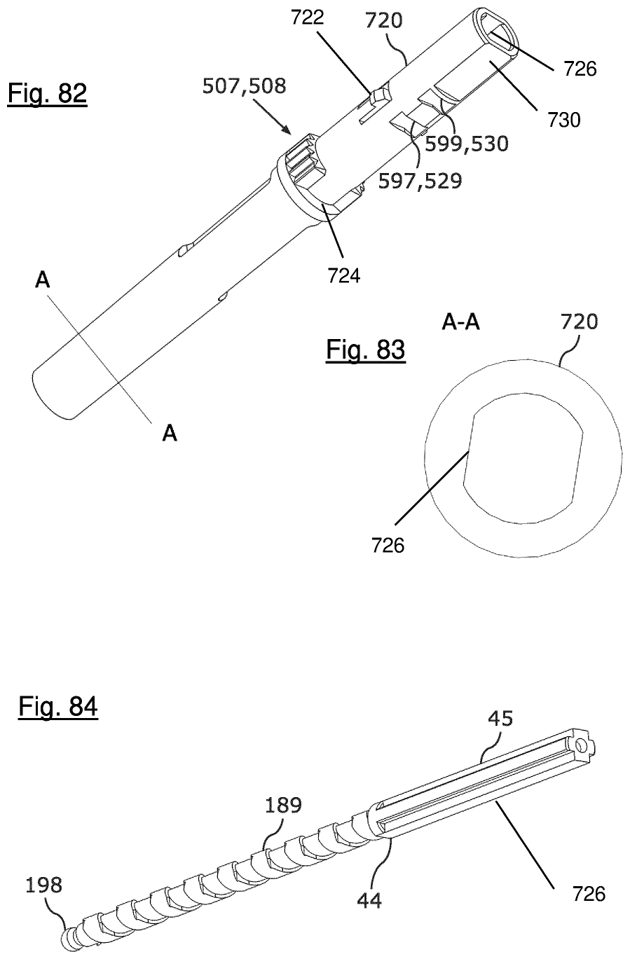

FIG. 82 shows a perspective view of the coupling element 720 of the dose delivery mechanism 754 and FIG. 83 shows a radial cut through the coupling element 720 along the line A-A shown in FIG. 82. The coupling element 720 is configured as a tubular member that extends along the longitudinal direction. It has a non-circular inner cross-section that is configured to receive the piston rod 44. The piston rod 44, which is shown in FIG. 84, has a distal section 45 having an outer shape that is configured to engage with the non-circular inner cross-section of the coupling element 720 to rotationally lock the piston rod 44 and the coupling element 720 and to allow axial movement between the coupling element 720 and the piston rod 44. The outer shape of the piston rod 44 at its distal end and the inner shape of the opening within the coupling element 720 form a rotational lock 726 between the coupling element 720 and the piston rod 44.

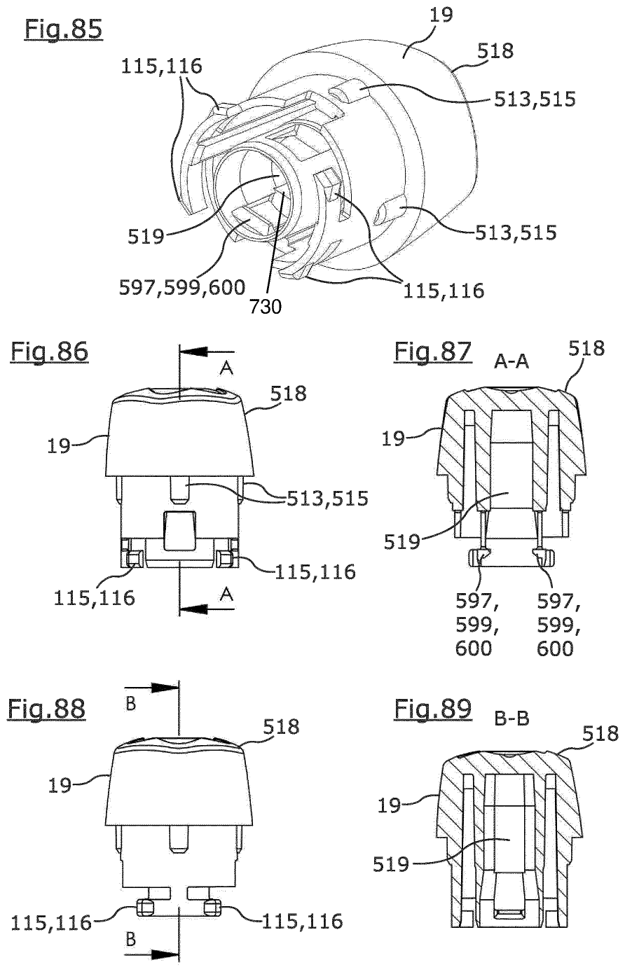

FIGS. 85 to 89 depict the adjusting element 518 of the dose delivery mechanism 754. The adjusting element 518 engages with a distal part of the coupling element 720. The adjusting element 518 thereby is rotationally fixed with respect to the coupling element 720 both in the preassembled state and in the assembled state of the dose delivery mechanism 704. A rotational lock 730 between the coupling element 720 and the adjusting element 518 comprises a non-circular outer cross section of the coupling element 720 that matches and engages with a corresponding inner shape of a central opening 519 of the adjusting element 518.

A latching mechanism 597 acts between the coupling element 720 and the adjusting element 518. In the assembled state of the dose delivery mechanism 754, the latching mechanism 597 prevents the adjusting element 518 from moving distally from the assembled position into the preassembled position with respect to the housing 532. The latching mechanism 597 comprises latch parts 600 formed at the adjusting element 518 and latch counterparts 529 formed at the coupling element 720. The latch parts 600 are configured as flexible hooks that protrude radially inward from the inner surface of the adjusting element 518 at the opening 519. The latch counterparts 529 are configured as recesses located at the outer surface in the distal part of the coupling element 720.

The coupling element 720 can form a retaining member of the dose delivery mechanism 754.

In the preassembled state of the dose delivery mechanism 754, the adjusting element 518 is located at a more distal position with respect to the coupling element 720 than in the assembled state. The more distal position is a preassembled position or first axial position of the adjusting element 518 with respect to the coupling element 720. In this position, the adjusting element 518 is prevented from being detached from the dose delivery mechanism 754 and the coupling element 720 by a further latching mechanism 599. A further latch part of the further latching mechanism 599 is formed by the latch part 600 and a further latch counterpart 530 of the further latching mechanism 599 is formed by an additional recess at the outer surface of the coupling element 720. The further latch counterpart 530 is thereby located at a distal side from the latch counterpart 529.

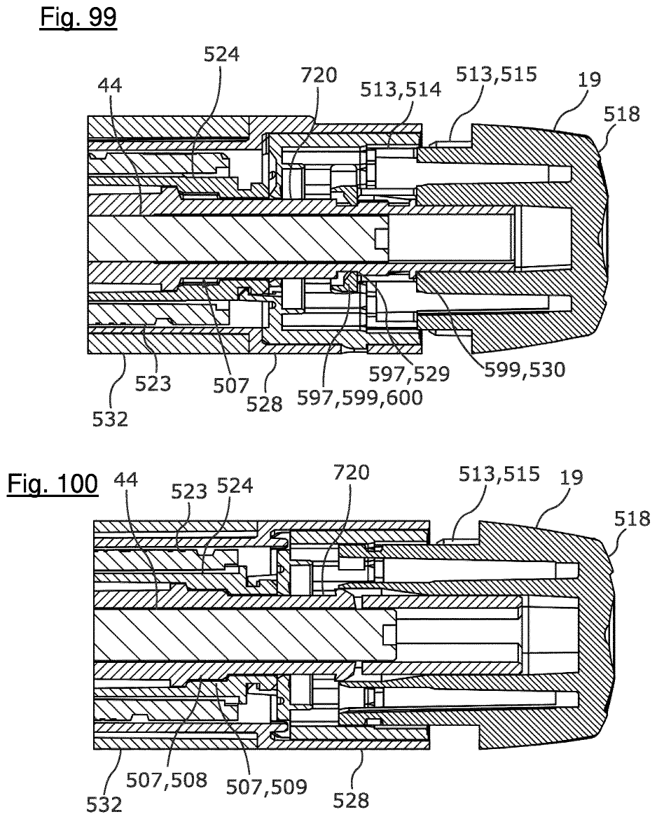

In the assembled state shown in FIGS. 99 to 102 and described below, the adjusting element 518 is in a proximal position or second axial position with respect to the coupling element 720. The proximal position is the assembled position of the adjusting element 518.

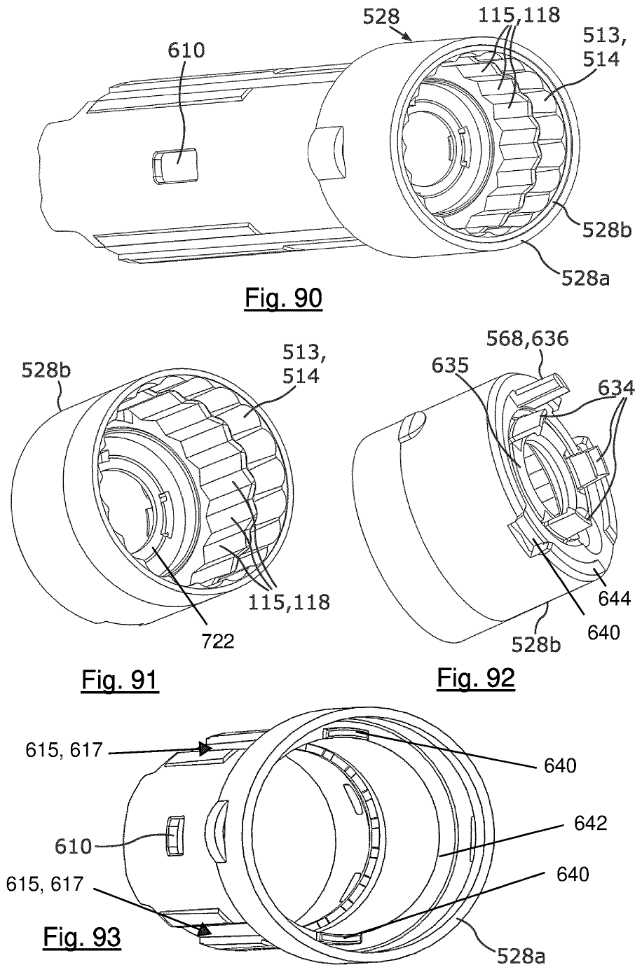

FIGS. 90 to 93 show a sleeve 528 of the dose delivery mechanism 754 that is rotationally fixed and axially movable with respect to the housing 532. The sleeve 528 comprises longitudinal recesses on its outer surface that engage with corresponding longitudinal ridges on an inside surface of the housing 532 to rotationally fix the sleeve 528 to the housing 532. The sleeve 528 comprises an outer part 528a and insert 528b that is rotationally and axially fixed within the outer part 528a at a distal end of the outer part 528a.

A dose definition mechanism 115 of the dose delivery mechanism 754 acts between the adjusting element 518 and the sleeve 528. The dose definition mechanism 115 comprises engagement features 116 that are configured as flexible hooks and provided at a proximal end of the adjusting element 518. The engagement features 116 interact with dose stops 118 provided in a proximal part of an inside surface of the insert 528b of the sleeve 528.

Furthermore, the dose delivery mechanism 754 comprises a clutch mechanism 513 that acts between the adjusting element 518 and the sleeve 528. The clutch mechanism 513 comprises teeth 515 that are located at a proximal outer surface of the adjusting element 518. When closing the clutch mechanism 513, the teeth 515 engage with corresponding teeth 514 provided in a distal part of the inside surface of the insert 528b. The inside surface thereby is a side surface of a cavity formed at the distal end of the insert 528b and the sleeve 528.

The adjusting element 518 forms a first clutch member of the clutch mechanism 513 and the sleeve 528 forms a second clutch member of the clutch mechanism 513. The housing 532 of the dose delivery mechanism 754 forms an additional member of the dose delivery mechanism 754 to which the adjusting element 518 is rotationally coupled during dose setting in the assembled state and from which the adjusting element 518 is rotationally decoupled during dose delivery in the assembled state. Furthermore, the clutch mechanism 513 forms a locking mechanism that rotationally locks the adjusting element 718 to the housing 532 during dose delivery in the assembled state of the dose delivery mechanism 754.

FIGS. 94 to 96 show a coupling member 524 of the dose delivery mechanism 754. The coupling member 524 is axially fixed to the sleeve 528 by a rim 592 provided at a distal end of the coupling member 524. The rim 592 is held between proximal blocking elements 634 and a distal blocking element 635 provided at a proximal end of the insert 528b. The proximal blocking elements 634 are configured as flexible hooks and the distal blocking element 635 is formed by a radial surface of the insert 528b.

A clutch mechanism 507 acts between the coupling member 524 and the coupling element 720, which is received within the coupling member 524. The clutch mechanism 507 comprises a first clutch part 508 that is located on an outside surface of the coupling element 720 and that comprises longitudinal teeth. The clutch mechanism 507 further comprises a second clutch part 509, which is located on an inside surface of the coupling member 524. The second clutch part 509 is configured as longitudinal teeth that mesh with the longitudinal teeth of the first clutch part 508 in the closed state of the clutch.

The coupling element 520 forms a first clutch member of the clutch mechanism 507 and the coupling member 524 forms a second clutch member of the clutch mechanism 507. The dosing member 523 of the dose delivery mechanism 754 forms a further member of the dose delivery mechanism 754 to which the adjusting element 518 is rotationally coupled during dose setting in the assembled state and from which the adjusting element 518 is rotationally decoupled during dose delivery in the assembled state.

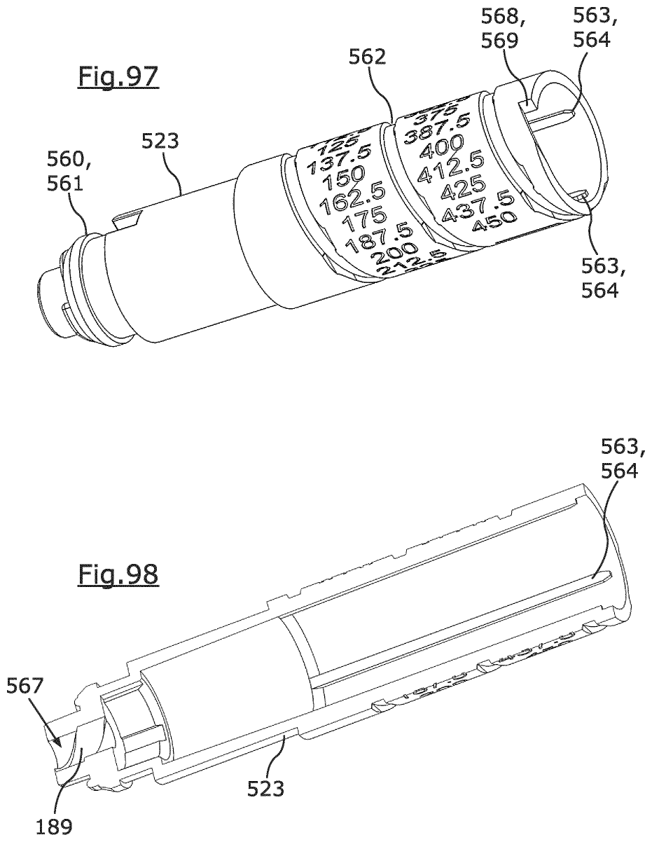

FIGS. 97 and 98 show a dosing member 523 of the dose delivery mechanism 754. A radial stop 568 is provided between the dosing member 523 and the insert 528b of the sleeve 528. The radial stop 568 comprises a stop surface 636 at a proximal extension of the insert 528b and a corresponding stop surface 569 at the distal end of the dosing member 523.

As can be seen from FIGS. 99 and 100, which show the dose delivery mechanism 754 in the assembled state during dose setting with no dose set, the clutch mechanism 507 between the coupling element 720 and the coupling member 524 is closed and the clutch mechanism 513 between the adjusting element 518 and the sleeve 528 is opened during dose setting. Dose setting is then effected by rotating the adjusting element 720 in the same way as it is described for the third injection pen 500.

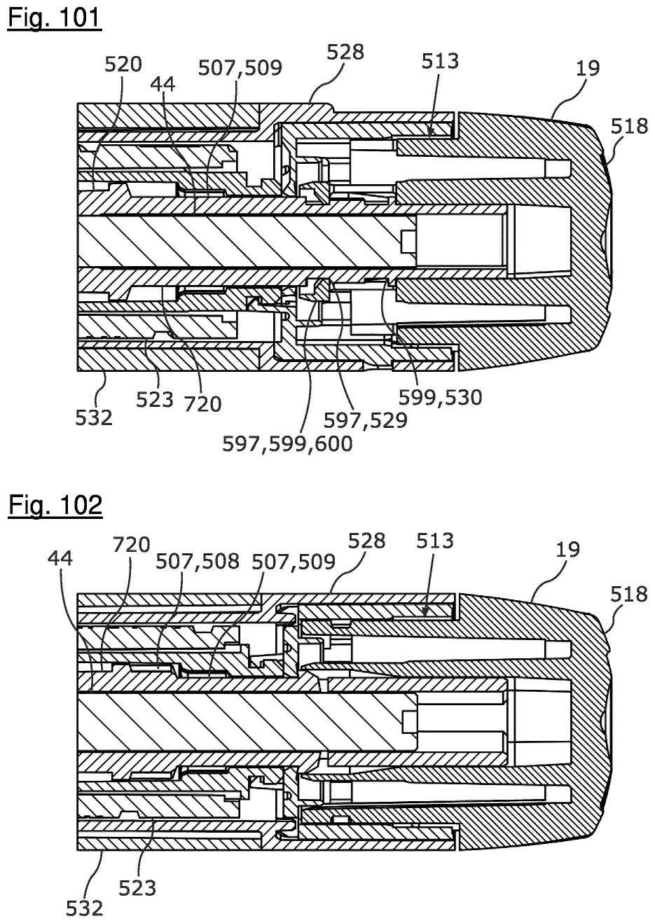

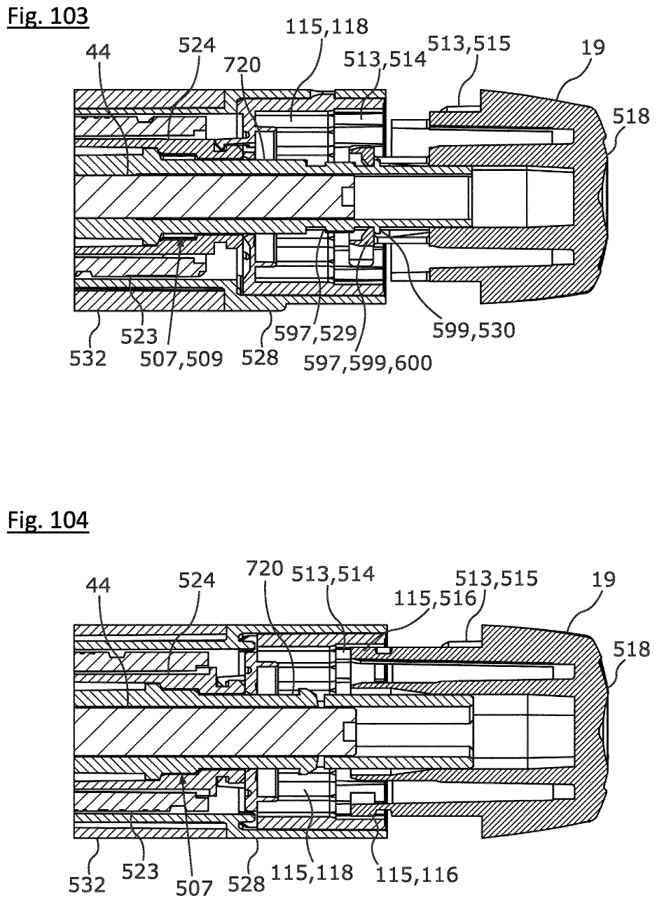

FIGS. 101 and 102 depict the dose delivery mechanism 754 in the assembled state during dose delivery when a set dose has been completely expelled and the user still pushes the adjusting element 518 in the proximal direction 1. During dose delivery, the clutch mechanism 507 is opened, thus allowing rotation between the piston rod 44 and the dosing member 523, and the clutch mechanism 513 is closed, thus rotationally locking the piston rod 44 to the housing 532.

FIGS. 103 and 104 depict the dose delivery mechanism 754 in the preassembled state. Compared to the assembled state, the adjusting element 518 is shifted in the distal direction with respect to the coupling element 720 and the housing 532. The latch part 600 of the adjusting element 518 then engages with the further latch counterpart 530 provided distally from the latch counterpart 529 at the coupling element 720. In the state depicted in FIGS. 103 and 104, the adjusting element 518 is positioned in a preassembled position with respect to the housing 532. The spring acting between the adjusting element 518 and the sleeve 528 biases the adjusting element 518 into the preassembled position. The preassembled position forms a rest position of the adjusting element 518 when the dose delivery mechanism 754 is in the preassembled state. The adjusting element 518 takes up the preassembled position or rest position when none of the components of the dose delivery mechanism 754 is actuated.

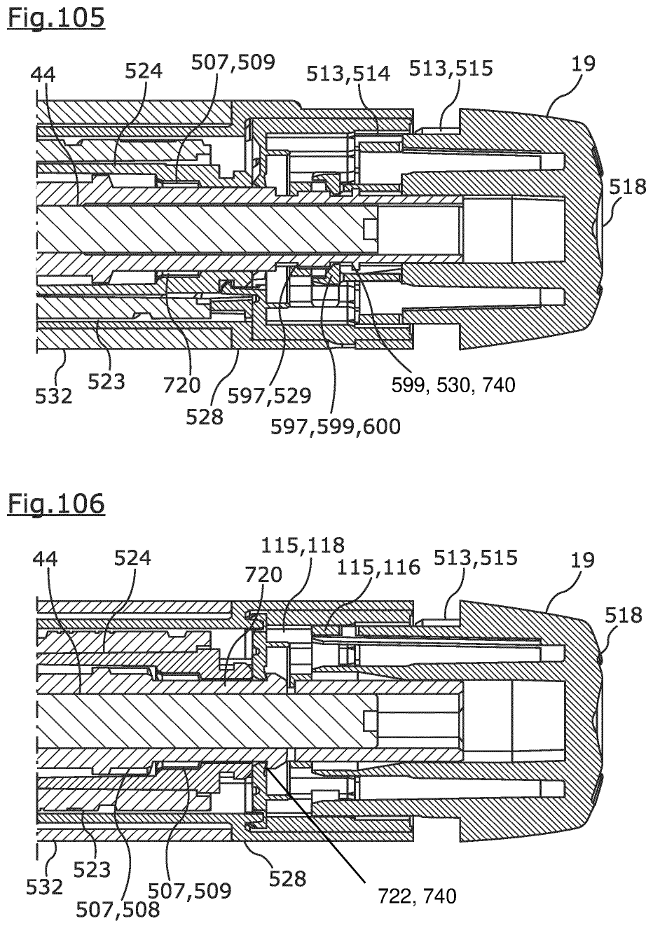

To adjust the position of the piston rod 44 in the preassembled state, the adjusting element 518 is pushed in the proximal direction 1 from the preassembled position into an adjusting position against the biasing force of the spring acting between the adjusting element 518 and the sleeve 528, as it is depicted in FIGS. 105 and 106. Proximal movement of the adjusting element 518 results in proximal movement of the coupling element 720 and in opening of the clutch mechanism 507. At the same time, the clutch mechanism 513 remains opened and the teeth 515 of the adjusting element 518 are prevented from engaging with the teeth 514 of the sleeve 528. This is due to an axial offset between the teeth 513 and the teeth 514.

When an assembler of the fourth injection pen 700 rotates the adjusting element 518 in the adjusting position depicted in FIGS. 105 and 106, the piston rod 44 rotates together with the adjusting element 518 and the dosing member 523 does not rotate due to the opened clutch mechanism 507. Rotation of the piston rod 44 with respect to the dosing member 523 then axially moves the piston rod 44 with respect to the housing 532 via the threaded connection 189.

With the adjusting element 518 in the adjusting position, the dose definition mechanism 115 is active. The engagement features 116 of the adjusting element 518 thereby engage with the dose stops 118. Rotation of the adjusting element 518 in the adjusting position therefore causes the dose definition mechanism 115 to provide a feedback to the user when the engagement features 116 ride over the dose stops 118.

The adjusting element 518 is configured to be moved from the adjusting position shown in FIGS. 103 and 104 in the proximal direction 1. During this movement, the coupling element 720 remains axially stationary since an axial stop 722 acting between the coupling element 720 and the sleeve 528b blocks proximal movement of the adjusting element 518. Movement of the adjusting element 518 then disengages the further latching mechanism 599 and the adjusting element 518 moves in the proximal direction 1 until the latching mechanism 597 engages. This irreversibly fixes the adjusting element 518 in its proximal position or second axial position with respect to the coupling element 720.

When the adjusting element 518 is released after positioning it in its proximal position, the adjusting element 518 is biased in the distal direction with respect to the housing 532 and with respect to the sleeve 528 until it reaches its most distal position with respect to the sleeve 528 in the assembled state. The adjusting element 518 then is in its dose setting position.

The axial stop 722 that limits proximal movement of the coupling element 720 acts between the coupling element 720 and the insert 528b. The coupling element 720 engages the insert 528b via the axial stop 722. As it is shown in FIG. 82, the axial stop 722 comprises a stop surface at the coupling element 720. The stop surface is exemplarily provided at a protrusion on the outside surface of the coupling element 720. The axial stop 722 further comprises a further stop surface provided at the insert 528b, as shown in FIG. 91. The further stop surface is exemplarily provided at an inside surface of the insert 528b. It is configured as an annular surface surrounding an opening that receives the coupling element 720.

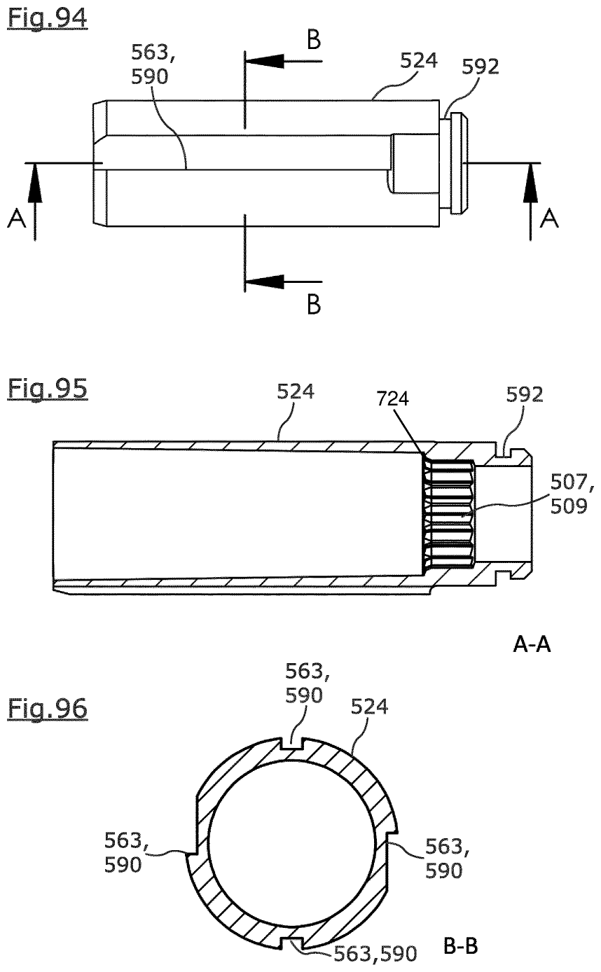

A further axial stop 724 acts between the coupling element 720 and the coupling member 524. The coupling element 720 engages the coupling member 524 via the further axial stop 724. As it is shown in FIG. 82, the further axial stop 724 comprises a stop surface at the coupling element 720. The stop surface is exemplarily provided at a protrusion on the outside surface of the coupling element 720, namely on an annular outer rim. The further axial stop 724 further comprises a further stop surface provided at the coupling member 524, as shown in FIG. 95. The further stop surface is exemplarily provided at an inside surface of the coupling member 524. It is configured as an annular surface surrounding an opening that receives the coupling element 720. The further stop surface is provided by an axial end surface of the clutch mechanism 507.

The axial stop 722 is configured as a latch stop. One of the stop surface and the further stop surface, exemplarily the stop surface, is provided at a flexible element that is configured to radially bend. This allows the other one of the stop surface and the further stop surface, exemplarily the further stop surface, to pass the one of the stop surface and the further stop surface in one direction, exemplarily in the proximal direction 1, during assembly, whereby movement in the opposite direction, exemplarily in the distal direction, is blocked after assembly.

In the preassembled state shown in FIGS. 103 and 104, the adjusting element 518 is in a first axial position with respect to the retaining member formed by the coupling element 720. Furthermore, in the assembled state shown in FIGS. 99 to 102, the adjusting element 518 is in a second axial position with respect to the retaining member 720. The first axial position thereby is a more distal axial position than the second axial position. In the assembled state, the adjusting element 518 is in the second axial position with respect to the retaining member 720 at least during dose setting, namely during dose setting and dose delivery.

As can be seen from FIGS. 105 and 106, a stop 740 is formed between the adjusting element 518 and the housing 532, namely between the adjusting element 518 and the sleeve 528. The stop 740 resists against further proximal movement of the adjusting element 518 in the adjusting position shown in FIGS. 105 and 106. It thereby exerts a resistance against the movement of the adjusting element 518 into the proximal direction 1. Exemplarily, the stop 740 is formed by the axial stop 722 and the further latching mechanism 599. Thereby, the coupling element 720 exemplarily forms part of the stop 740.

The resistance exerted by the stop 740 can be overcome by pushing the adjusting element 518 in the proximal direction with a force that is larger than the resistance exerted by the stop 740. Thereby, the resistance is determined by the resistance exerted by the further latching mechanism 599. When the resistance is overcome, the adjusting element 518 further moves in the proximal position until it reaches the second axial position with respect to the coupling element 720 shown in FIGS. 99 to 102.

With the dose delivery mechanisms 554, 754, the adjusting element 518 forms a knob. The knob is placed at the distal end of the dose delivery mechanisms 554, 754 and forms the distal end of the dose delivery mechanisms 554, 754. The knob is configured to be gripped and rotated by a user of the dose delivery mechanisms 554, 754 to set a dose in the assembled state.

With the fourth injection pen 700, the piston rod 44 can be configured to advance by a first distance into the proximal direction 1 upon proximal movement of the actuation member 518 by a second distance, wherein the second distance is less than 1.5 times the first distance. With the fourth injection pen 700, the ratio between the second distance and the first distance is given by the ratio of the pitches of the threaded connection 189 between the piston rod 44 and the dosing member 523 and of the further threaded connection 562 between the sleeve 528 and the dosing member 523.

Additionally or alternatively, the fourth injection pen 700 can also comprise the drug reconstitution unit 56 of the first injection pen 10. Such a fourth injection pen 700 then can be configured to receive the double chambered cartridge 48 and to perform reconstitution of a lyophilized drug prior to drug delivery.

Embodiments of the fourth injection pen 700 can be configured, like the first injection pen 10, to restrain a user from prematurely activating the fourth injection pen 700. Embodiments of the fourth injection pen 700 can be configured, like the first injection pen 10, to axially lock the actuation member 520 with respect to the housing 532 prior to setting and delivering a first dose. Like the first injection pen 10, the fourth injection pen 700 can comprise the knob cover 16 and/or the knob key 30.

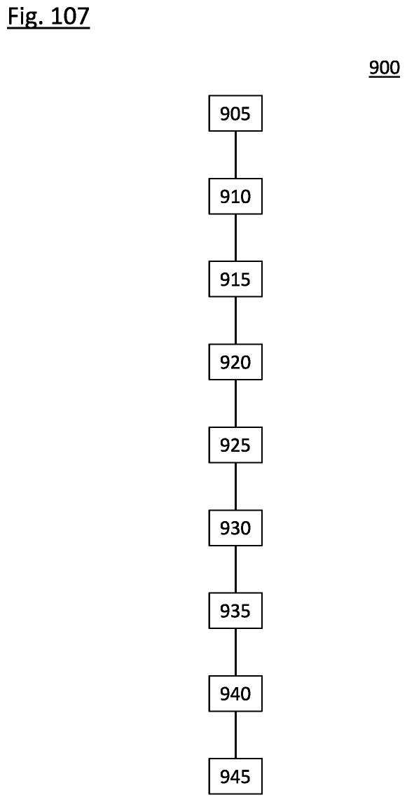

FIG. 107 depicts an exemplary method 900 for at least partly assembling the dose delivery mechanism 754.

The method 900 comprises as a first step 905 providing the housing 532. For example, the housing 532 can be provided in an assembly jig.

As a second step 910, the method 900 comprises inserting the outer part 528a of the sleeve 528 into the housing 532. The outer part 528a thereby is inserted from the distal side into the housing 532. Furthermore, the outer part 528a is positioned axially in its zero dose position, which is the most proximal position of the outer part 528a with respect to the housing 532. In the inserted position, the outer part 528a rests against the housing 532, which prevents further movement of the outer part 528a in the proximal direction 1.

Prior to inserting the outer part 528a, the method 900 comprises rotationally aligning the outer part 528a with respect to the housing 532 so that components of a rotational lock 615 that rotationally fixes the outer part 528a to the housing 532 are aligned with each other along the longitudinal axis. As can be seen from FIGS. 80 and 93, the rotational lock 615 comprises mating components provided at an outside surface of the outer part 528a and an inner surface of the housing 532. The mating components form a splined connection. Exemplarily, the mating components comprise longitudinal ridges 536 at the housing 532 and longitudinal grooves 617 at the outer part 528a. with other embodiments, the ridges can be provided at the outer part 528a and grooves can be provided at the housing 532.

As a third step 915, the method 900 comprises placing the dosing member 523 within the outer part 528a. The dosing member 523 is inserted into the outer part 528a from the distal side and in the proximal direction. The dosing member 523 is thereby engaged with the outer part 528a via the thread 562 and screwed into the outer part 528a. The dosing member 523 is positioned axially in its zero dose position with respect to the housing 532, whereby the zero dose position is an axial position in which the dosing member 523 has reached the rotatable fixation 560 and is axially fixed with respect to the housing 532. Furthermore, the dosing member 532is in a rotational position with respect to the housing 532, in which the thread 562 that couples the dosing member 523 to the outer part 528a is orientated in a rotational position in which the outer part 528a is positioned in the zero dose position and in its most proximal position with respect to the housing 532.

As a fourth step 920, the method 900 comprises providing a subassembly. The subassembly comprises the coupling element 720, the coupling member 524 and the insert 528b of the sleeve 528. The coupling member 524 thereby is connected to the insert 528b by an axially fixed and rotationally movable connection. This connection is formed by the annular rim 592 of the coupling member 524, see, for example, FIGS. 94 and 95, being held in between the at least one proximal blocking element 634 and the distal blocking element 635 provided at the insert 528b, namely at the proximal end of the insert 528b, see FIG. 92.

Furthermore, the coupling element 720 of the subassembly is axially restrained with respect to the insert 528b in the subassembly. It thereby can move between a proximal position and a distal position along the longitudinal axis. Further distal movement from the distal position is blocked by the axial stop 722 and further proximal movement from the proximal position is blocked by the further axial stop 724.

With the subassembly, the coupling member 524 and the insert 528b are axially connected to each other and the coupling element 720 is received within the coupling member 524 and the insert 528b.

The subassembly is provided by first providing the coupling element 720 and by then connecting the coupling member 524 to the coupling element 720. The coupling member 524 is thereby connected to the coupling element 720 from the distal side. The coupling member 524 is moved axially with respect to the coupling element 720 in the proximal direction 1, until further proximal movement of the coupling member 524 with respect to the coupling element 720 is blocked, exemplarily by the further axial stop 724.

During axial movement of the coupling member 524 with respect to the coupling element 720, the stop surface of the axial stop 722 provided at the coupling element 720 is deflected radially inward in the radial direction to allow for passage of the further stop surface of the axial stop 722 provided at the coupling member 524.

The insert 528b is then connected to the coupling member 524. The insert 528b thereby is connected from the distal side of the coupling member 524. It is moved proximally with respect to the coupling member 524 until further axial movement is stopped, exemplarily by the axially fixed and rotationally movable connection 592, 634, 635 between the coupling member 524 and the insert 528b.

As a fifth step 925, the method 900 comprises attaching the subassembly to the housing 532. Thereby, the coupling element 720 and the coupling member 524 are inserted into the housing 532. Furthermore, the coupling element 720, the coupling member 524 and the insert 528b are received within the outer part 528a of the sleeve 528. In addition, the coupling member 524 is inserted into the dosing member 523.

Prior to attaching the subassembly to the housing 532, the coupling member 524 is rotationally aligned with respect to the housing 532 and the dosing member 523 within the housing 532. This brings the components of the rotation fixation 563 between the dosing member 523 and the coupling member 524, namely the recesses 590 and the longitudinal ridges 564, into rotational alignment.

Furthermore, the insert 528b is rotationally aligned with respect to the housing 532 and the outer part 528a of the sleeve 528. This aligns a rotational lock 640 which is provided between the insert 528b and the outer part 528a of the sleeve 528, see FIGS. 92 and 93.

The subassembly is attached to the housing 532 from the distal side of the housing 532. The subassembly thereby is moved in the proximal direction 1 until further proximal movement is stopped by engagement between the insert 528b and the outer part 528a. Thereby, a proximal facing surface 644 of the insert 528b engages with a distally facing surface 642 of the outer part 528a, see FIGS. 92 and 93.

Attachment of the subassembly to the housing 532 positions the coupling member 524 and the insert 528b axially into their respective zero dose position with respect to the housing 532. The zero dose position of the coupling member 524 is its most proximal position with respect to the housing 532 in the assembled state. Furthermore, the zero dose position of the insert 528b is also its most proximal position with respect to the housing 532 in the assembled state.

With alternative embodiments, the subassembly can also comprise only the coupling member 524 and the coupling element 720. The coupling member 524 and the coupling element 720 thereby can have been assembled as described before. For example, the coupling member 524 is axially restrained with respect to the coupling element 720 via the axial stop 722 and the further axial stop 724. Thereby, the axial stop 722 acts between the coupling element 720 and the coupling member 524 by the stop surface of the further axial stop 722 provided at the coupling element 722 engaging with the distal facing end surface of the coupling member 524.

The method 900 then can comprise a step of attaching the subassembly consisting of the coupling member 524 and the coupling element 720 to the housing 532, analogously to the step described before. After having attached the subassembly to the housing 532, the method 900 then can comprise a step of attaching the insert 528b to the housing 532, as it is described before. The method 900 then can comprise an additional step of pulling the coupling element 720 together with the coupling member 524 in the distal direction to attach the coupling member 524 to the insert 528b via the axially fixed and rotationally movable connection 592, 634, 635.

As a sixth step 930, the method 900 comprises attaching the piston rod 44 to the housing 532. Thereby, the piston rod 44 is inserted from the distal end of the housing 532 into the housing 532 in the proximal direction 1. Furthermore, the piston rod 44 is inserted into the coupling element 720 at the distal end of the coupling element 720. Prior to or upon insertion, the piston rod 44 is rotationally aligned with respect to the coupling element 720. This aligns the components of the rotational lock 726 between the piston rod 44 and the coupling element 720 with respect to each other. The piston rod 44 can be moved in the proximal direction until it reaches a predetermined axial position with respect to the housing 532. This position can, for example, be given by a stop surface of an assembly jig holding the housing 532.

During attachment, the piston rod 44 is screwed into the dosing member 523 via the threaded connection 189.

As a seventh step 935, the method 900 comprises positioning the spring at the sleeve 528, which spring acts between the adjusting element 518 and the sleeve 528 in the assembled state. The spring thereby is inserted into the distal opening of the sleeve 528, which opening is provided at the insert 528b.

As an eighth step 940, the method 900 comprises attaching the adjusting element 518 to the housing 532. The adjusting element 518 thereby is being attached to the coupling element 720. It is being attached in the proximal direction from the distal end of the housing 532 and the coupling element 720. Prior to or upon attachment, the adjusting element 518 is rotationally aligned with respect to the housing 532. Furthermore, the adjusting element 518 is rotationally aligned with respect to the coupling element 720. This aligns the rotational lock 730 between the coupling element 720 and the adjusting element 518.

The adjusting element 518 is attached to the coupling element 720 in the preassembled position with respect to the coupling element 720. Furthermore, the adjusting element 518 is attached to the coupling element 720 in the preassembled position with respect to the housing 532. Thereby, the latch part 600 of the adjusting element 518 engages with the further latch counterpart 530 of the further latching mechanism 599 between the coupling element 720 and the adjusting element 518.

As a ninth step 945, the method 900 comprises attaching the bearing 46 to the piston rod 44.

All remaining components except for the adjusting element 518, the piston rod 44 and components axially fixed to the piston rod 44, such as the bearing 46, are then axially positioned in their respective zero dose position with respect to the housing 532.

With the method 900, the first step 905 up to the ninths step 945 are performed in ascending order starting from the first step 905 and ending with the ninth step 945.

With alternative embodiments, the bearing 46 can also be attached to the piston rod 44 upon attaching of the piston rod 44 to the housing 532. For example, the bearing 46 can be held together with the housing 532 in a fixed axial position with respect to the housing 532. The housing 532 and the bearing 46 thereby can be placed together in an assembly jig. The method 900 then can comprise a step of providing the bearing 46. This step can, for example, be the first step of the method 900. The bearing 46 thereby can be placed into the assembly jig. As a second step, the method 900 then can comprise the step of providing the housing 532. Exemplarily, the housing 532 can be provided by placing it into the assembly jig.

With other embodiments, the bearing 46 can be attached to the piston rod 44 at any time after having attached the piston rod 44 to the housing 532. Upon attachment of the piston rod 44 to the housing 532, the piston rod 44 is moved through the proximal opening within the housing 532.

Additionally or alternatively, the spring that acts between the adjusting element 518 and the sleeve 528 can also first be positioned at the adjusting element 518 and the spring and the adjusting element 518 then can be attached together to the housing 532. The spring thereby can be placed around an inner cylindrical section of the adjusting element 518 that has the engagement feature 116 of the dose definition mechanism 115 and the latch part 600 of the latching mechanisms 597, 599 at its proximal end, see FIG. 87.

The mechanism can comprise a dose definition mechanism that allows a user of the device to set at least one dose of medicament for delivery. For example, the dose definition mechanism can be configured to allow only a single predetermined dose to be set. Alternatively, the dose definition mechanism can also be configured to allow a multitude of differing predetermined doses to be set by the user, such as two or more differing doses.

With the injection pens 10, 300, 500, 700, the dose delivery mechanisms 54, 354, 554, 754 each comprises a dose definition mechanism 115, wherein the respective dose definition mechanism 115 acts between the respective dose setting element 18, 318, 518, and the housing 32, 332, 532 during dose setting. The dose definition mechanism 115 thereby has at least one dose stop 118 and a counter element 116, wherein the counter element 116 is configured to rotate with respect to the dose stop 118 when the dose setting element 38, 318, 518 rotates during dose setting and wherein the counter element 116 is configured to engage the dose stop 118 when the dose has been set. The counter elements 116 are formed by the respective engagement features 116 of the dose delivery mechanisms 54, 354, 554, 754.

The dose definition mechanism 115 can define the rotational positions of the dose setting element 22, 518 with respect to the housing 32, 332, 532 that correspond to settable doses. For each settable dose, the dose definition mechanism 115 can comprise a separate dose stop 118. The dose setting element 22, 518 then can be configured to only perform less than a full rotation during dose setting. With the third and fourth injection pen 500, 700, the dose definition mechanisms 115 can also comprise individual dose stops 118 that define more than a single dose, for example, the individual dose stops 118 can be configured to engage with the counter element 116 once upon each full rotation of the counter element 116 with respect to the dose stops 118.

The counter element 116 can be configured as a flexible element that snaps over the dose stop 118 upon setting the dose. For example, the counter element 116 can be configured as a flexible protrusion at a component of the respective dose delivery mechanisms 54, 354, 554, 754. The counter element 116 can, for example, be integrally formed with the component of the respective dose delivery mechanisms 54, 354, 554, 754 it is fixed to. With the first and second injection pen 10, 300, said component is formed by the dosing member 23, 323, with the third and fourth injection pen 500, 700, it is formed by the dose setting element 518.

With the first and second injection pen 10, 300, engagement of the counter element 116 with the dose stop 118 prevents the spring 40 from releasing the energy stored upon rotation of the dose setting element 22 during dose setting. The dose definition mechanism 115 thereby can provide a latching function that keeps the spring 40 in a tensioned state until the dose is delivered by transferring the respective dose delivery mechanism 54, 354 from the dose setting state to the dose delivery state.

With the first and second injection pen 10, 300, the counter element 116 is configured to disengage from the dose stop 118 upon transfer of the respective dose delivery mechanism 54, 354 from the dose setting state into the dose delivery state. This prevents the dose definition mechanism 115 from interfering with the delivery of the set dose. Furthermore, it can allow the spring 40 to release the energy stored upon rotation during dose setting. With embodiments having more than a single dose stop 118, the counter element 116 can be configured to disengage from all dose stops 118 upon transfer of the respective dose delivery mechanisms 54, 354 into the dose delivery state. This can allow the counter element 116 to rotate back to its initial position during dose delivery without interfering with the dose stops 118. The initial position can correspond to a zero dose position in which no dose has been set.

For example, the counter element 116 can be configured to disengage from the dose stop 118 by axially moving with respect to the dose stop 118.

With the injection pens 10, 300, 500, 700, one of the dose stop 118 and the counter element 116, such as the dose stop 118, is rotationally fixed with respect to the housing 32, 332, 532. The one of the dose stop 118 and the counter element 116 then can be axially movable with respect to the dose setting element 22, 518.

With the first and second injection pen 10, 300, the one of the dose stop 118 and the counter element 116, such as the dose stop 118, is axially fixed with respect to the button 18, 318. This allows to move the one of the dose stop 118 and the counter element 116 together with the button 18, 318 upon transfer of the respective dose delivery mechanism 54, 354 from the dose setting state into the dose delivery state. The one of the dose stop 118 and the counter element 116 than can disengage from the other one of the dose stop 118 and the counter element 116 by this movement. For example, the one of the dose stop 118 and the counter element 116 can be linearly guided at the housing 32, 332.

With some embodiments, the one of the dose stop 118 and the counter element 116 is fixed to an outer housing part of the respective dose delivery mechanism 54, 354, 554, 754. The outer housing part can be fixed to a connection for coupling a medicament container 48, 348 to the respective dose delivery mechanism 54, 354, 554, 754. Alternatively, the outer housing part can also be movable with respect to the connection, such as axially movable. Additionally, the outer housing part can be rotationally fixed with respect to the connection. For example, the outer housing part can be a housing connector, such as a housing connector that engages with the housing 32, 332, 532 via an axially movable and rotationally fixed connection. With the first and second injection pen 10, 300, the outer housing part is formed by the dose selector 28, with the third and fourth injection pen 500, 700, it is formed by the sleeve 528.

With the first and second injection pen 10, 300, the dose definition mechanism 115 acts between the dosing member 23, 332 and the housing 32, 332, 532. It then defines rotational positions of the dosing member 23, 332 with respect to the housing 32, 332 that correspond to settable doses.

With the first and second injection pen 10, 300, the other one of the dose stop 118 and the counter element 116, such as the counter element 116, is rotationally fixed with respect to the dosing member 23, 332. Exemplarily, the other one of the dose stop 118 and the counter element 116 is permanently rotationally fixed with respect to the dosing member 23, 332. The other one of the dose stop 118 and the counter element 116 is exemplarily additionally fixed to the dosing member 23, 332, such as to the first part of the dosing member 23, 332 that is movable with respect to the housing 32, 332, 532.

With the first, second, third and fourth injection pen 10, 300, 500, 700, the other one of the dose stop 118 and the counter element 116, such as the counter element 116, is axially movable with respect to the button 18, 318, 518. The one of the dose stop 118 and the counter element 116, such as the dose stop 118, is axially fixed with respect to the button 18, 318, 518. This allows to disengage the dose stop 118 from the counter element 116 by moving the button with respect to the housing 32, 332, 532, as it is the case for the first and second injection pen 10, 300.

With the second injection pen 300, the dose delivery mechanisms 354 comprises a blocking mechanism having a first element and a second element, wherein the first element engages the second element upon release of the button 318 during dose delivery to prevent a transfer of the respective dose delivery mechanism 354 from the dose delivery state to the dose setting state. This keeps the respective dose delivery mechanism 354 in the dose delivery state and prevents a change of the dose setting upon an interruption of dose delivery due to the torque provided by the spring 40. The blocking mechanism can block distal movement of the button 318 against a biasing force biasing the button 318 in the distal direction. The biasing force can be provided by the biasing element 250.

The blocking mechanism is configured to disengage the first element from the second element at a zero dose position at which a set dose has been fully delivered. This allows the dose delivery mechanisms 354 to return to the dose setting state so that a subsequent dose can be set after having completed a previous medicament delivery.

Exemplarily, the first element rotates with respect to the second element in a first direction during dose setting and rotates in a second direction opposite the first direction during dose delivery. Relative movement between the first and second element can bring the first and second element in relative positions that prevent mutual engagement at the end of dose delivery and/or when a dose has been set.

With the second injection pen 300, the first element is configured as the circumferential rib 156 that longitudinally extends around an axis of the housing 332 and the second element is configured as a stop or counter element formed by the engagement feature 116 of the snap element 24 that travels along the circumferential rib 124 during dose delivery.

Exemplarily, the second element passes the first element upon release of the button 318 at the end of dose delivery. For example, the second element can pass through an opening within the first element. The second element can rotate into alignment with the opening at the end of dose delivery.

Additionally or alternatively, the blocking mechanism can be configured to prevent transfer of the dose delivery mechanism 354 from the dose setting state into the dose delivery state unless a dose has been set. Such a blocking mechanism is exemplarily also implemented in the dose delivery mechanism 54 of the first injection pen 10. Exemplarily, the second element passes the first element upon transfer of the dose delivery mechanism 54, 354 from the dose setting state into the dose delivery state. Exemplarily, the second element passes through one of the openings or cut-outs 158 within the first element. The second element exemplarily rotates into alignment with the opening 158 when a dose has been set.

Exemplarily, the second element passes through one of the openings or cut-outs 158 within the first element upon transfer of the mechanism from the dose setting state into the dose delivery state. Such openings 158 prevent blocking and thus allow axial movement of the button 18, 318 to initiate dose delivery.

Exemplarily, the first element of the blocking mechanism and one of the dose stop 118 and the counter element 116, such as the dose stop 118, are fixed to the same member of the dose delivery mechanism 54, 354. Furthermore, the second element of the blocking mechanism and the other one of the dose stop 118 and the counter element 116, such as the counter element 116, are fixed to the same further member of the dose delivery mechanism 54, 354. This allows to precisely align the elements of the blocking mechanism and the elements of the dose definition mechanism 115 and enhances reliability of the dose delivery mechanism 54, 354.

The member of the mechanism that comprises the first element of the blocking mechanism and the one of the dose stop 118 and the counter element 116 exemplarily is the dose selector 28 of the dose delivery mechanism 54, 354. The further member of the dose delivery mechanism 54, 354 that comprises the second element of the blocking mechanism and the other one of the dose stop 118 and the counter element 116 is exemplarily a carrier that is rotationally movable with respect to the dose selector 28. The carrier exemplarily is a part of the dosing member 23, 332, namely by the snap element 24 of the dosing member 23, 323.

The dose selector 28 exemplarily is at least partly located within an outer housing of the dose delivery mechanism 54, 354, namely the housing 32, 332. The dose selector 28 exemplarily is configured to protrude from the outer housing.

Exemplarily, one of the first element and second element of the blocking mechanism and one of the dose stop 118 and the counter element 118 are formed by a single element. As an example, the second element of the blocking mechanism and the counter element 116 of the dose definition mechanism 115 are formed by the single element. This facilitates alignment of the components of the blocking mechanism with respect to the components of the dose definition mechanism 115.

The single element exemplarily is a flexible element that is configured to snap over the dose stop 118 upon rotation with respect to the dose stop 118.

With the first and second injection pen 10, 300, the dose delivery mechanisms 54, 354 comprise a maximum dose mechanism that restrains further rotation of the dose setting element 22 upon dialing past a maximum dose setting, wherein the maximum dose mechanism comprises a maximum dose stop 126 that is exemplarily formed by the hard stop 128 and a blocking part 124 that is exemplarily formed by the hard stop 124 and wherein the blocking part 124 is configured to engage the maximum dose stop 126 128 upon dialing past the maximum dose setting. This provides a well-defined rotational end position of the dose setting element 22. The maximum dose stop 126 128 can also absorb a torque provided by a user and direct the torque to the housing 32, 332 of the dose delivery mechanism 54, 354.

The blocking part 124 exemplarily engages the maximum dose stop 126 128 right at the maximum dose setting. With other embodiments, the blocking part 124 can only engage the maximum dose stop 126 128 after having dialed past the maximum dose setting by a predefined amount.

The blocking part 124 exemplarily is configured as a hard stop that is rigidly connected, such as integrally formed, with a component of the mechanism. Likewise, the maximum dose stop 126 126 exemplarily is configured as such a hard stop.

Exemplarily, the maximum dose stop 126 and the blocking part 124 are configured to rotate with respect to each other during dose setting. As an example, one of the maximum dose stop 126 126 and the blocking part 124, namely the maximum dose stop 126 126, can be rotationally fixed with respect to the housing 32, 332 during dose setting and the other one of the maximum dose stop 126 126 and the blocking part 124, such as the blocking part 124, can be rotationally fixed with respect to the dose setting element 22 during dose setting. The other one of the maximum dose stop 126 126 and the blocking part 124 can be rotationally movable with respect to the dose setting element 22 during dose delivery.

Exemplarily, the maximum dose stop 126 is configured as a radial stop and the blocking part 124 is configured to rotate against the maximum dose stop 126 upon dialing past the maximum dose. Such a radial stop provides a well-defined rotational position in which the blocking part 124 and the maximum dose stop 126 get into engagement.

The maximum dose stop 126 and the blocking part 124 exemplarily comprise engagement surfaces that engage with each other. The engagement surfaces exemplarily are orientated essentially perpendicular, namely perpendicular, to a circumferential direction around the longitudinal axis of the dose delivery mechanism 54, 354.

Exemplarily, one of the maximum dose stop 126 and the blocking part 124, such as the maximum dose stop 126, is rotationally fixed with respect to the housing 32, 332. The one of the maximum dose stop 126 and the blocking part 124 exemplarily are permanently rotationally fixed with respect to the housing 32, 332, both during dose setting and dose de-livery.

Exemplarily, the one of the maximum dose stop 126 and the blocking part 124 is fixed to an outer housing part of the dose delivery mechanism 54, 354. The outer housing part is exemplarily configured as a housing connector that is located between the dose setting element 22 and the housing 32, 332 of the injection pen 10, 300. The outer housing part is configured as the dose selector 28.

Exemplarily, the other one of the maximum dose stop 126 and the blocking part 124, such as the blocking part 124, is rotationally fixed with respect to the dosing member 23, 332. The other one of the maximum dose stop 126 and the blocking part 124 then rotates with respect to the housing 332, 32 during both dose setting and dose delivery. This allows to reset the maximum dose mechanism during dose delivery. With embodiments, in which a rotational position of the dosing member 23, 332 defines the dose that has been set, rotationally fixing the one of the maximum dose stop 126 and the blocking part 124 to the dosing member 23, 332 precisely defines a maximum dose position in which the maximum dose stop 126 and the blocking part 124 engage with each other.

Exemplarily, the other one of the maximum dose stop 126 and the blocking part 124 is fixed to a coupling member that rotationally couples the dosing member 23, 332 to the dose setting element during dose setting. The coupling member is formed by the snap element 24.

Exemplarily, one of the dose stop 118 and the counter element 116, such as the dose stop 118, and one of the maximum dose stop 126 and the blocking part 124, such as the maximum dose stop 126, are fixed to the same member of the dose delivery mechanism 54, 354. This allows to precisely define the relative positions of the components of the dose definition mechanism 115 with respect to the components of the maximum dose mechanism. The member of the dose delivery mechanism 54, 354 exemplarily is the dose selector 28.

Exemplarily, the other one of the dose stop 118 and the counter element 116, such as the counter element 116, and the other one of the maximum dose stop 126 and the blocking part 124, such as the blocking part 124, are fixed to the same further member of the dose delivery mechanism 54, 354. This also allows to precisely define the relative positions of the components of the dose definition mechanism 115 with respect to the components of the maximum dose mechanism. The further member can, for example, be the carrier that is rotationally movable with respect to the dose selector 28 and that is formed by the snap element 24.

Exemplarily, the dose delivery mechanisms 54, 354 of the first and second injection pen 10, 300 comprise a zero dose mechanism that prevents further axial movement of the nut 38 at the end of dose delivery, wherein the zero dose mechanism comprises a zero dose stop and a further blocking part and wherein the further blocking part is configured to engage the zero dose stop at the end of dose delivery. This provides a well-defined end position for the piston rod 44 at the end of dose delivery and thus contributes to precisely define the amount of medicament delivered.

With the first injection pen 10, the zero dose stop is formed by the end stop 176 at the piston rod 44 guide 42 and the further blocking part is formed by the end stop 174 at the driver 36. With the second injection pen 300, the zero dose stop is formed as a protrusion on the inside surface of the dose selector 28 and the further blocking part is formed by the hard stop 124 at the snap element 24.

The further blocking part 124, 174 is exemplarily configured as a hard stop that is rigidly connected, such as integrally formed, with a component of the dose delivery mechanisms 54, 354. Likewise, the zero dose stop 176 exemplarily is configured as such a hard stop.

Exemplarily, the zero dose stop 176 and the further blocking part 124, 174 are configured to rotate with respect to each other during dose delivery. For example, one of the zero dose stop 176 and the further blocking part 124, 174, such as the zero dose stop 176, is rotationally fixed with respect to the housing 32, 332 during dose setting and the other one of the zero dose stop 176 and the further blocking part 124, 174, such as the further blocking part 124, 174, is rotationally fixed with respect to the dose setting element 22 during dose setting. The other one of the zero dose stop 176 and the further blocking part 124, 174 exemplarily is rotationally movable with respect to the dose setting element 22 during dose delivery.

Exemplarily, the zero dose stop 176 is configured as a radial stop and the further blocking part 124, 174 is configured to rotate against the zero dose stop 176 at the end of dose delivery. Such a radial stop provides a well-defined rotational position in which the further blocking part 124, 174 and the zero dose stop 176 become engaged with each other.

The zero dose stop 176 and the further blocking part 124, 174 exemplarily comprise engagement surfaces that engage with each other. The engagement surfaces can be orientated essentially perpendicular, such as perpendicular, to a circumferential direction around the longitudinal axis of the dose delivery mechanism 54, 354.

Exemplarily, one of the zero dose stop 176 and the further blocking part 124, 174, such as the zero dose stop 176, is rotationally fixed with respect to the housing 32, 332. The one of the zero dose stop 176 and the further blocking part 124, 174 exemplarily is permanently rotationally fixed with respect to the housing32, 332, both during dose setting and dose delivery.

Exemplarily, the one of the zero dose stop 176 and the further blocking part 124, 174 is fixed to an outer housing part of the dose delivery mechanism 54, 354. With the second injection pen 300, the outer housing part exemplarily is configured as a housing connector that is located between the dose setting element 22 and the housing 332 of the second injection pen 300. Also the first injection pen 10 can alternatively comprise such a zero dose stop 176.

Exemplarily, the other one of the zero dose stop 176 and the further blocking part 124, 174 is rotationally fixed with respect to the dosing member 23, 332. The other one of the zero dose stop 176 and the further blocking part 124 then rotates with respect to the housing 32, 332 during both dose setting and dose delivery. This allows to reset the zero dose mechanism during dose setting. With embodiments, in which a rotational position of the dosing member 23, 332 defines the dose that has been set, rotationally fixing the one of the zero dose stop 176 and the further blocking part 124, 174 to the dosing member 23, 332 precisely defines a zero dose position in which the zero dose stop and the further blocking part 124 engage with each other.

With the second injection pen 200, the other one of the zero dose stop 176 and the further blocking part 124, 174 is fixed to a coupling member that rotationally couples the dosing member 23, 332 to the dose setting member 22 during dose setting. The coupling member is formed by the snap element 24 of the dosing member 323. Also the first injection pen 10 can comprise such a configuration.

Exemplarily, one of the dose stop 118 and the counter element 116 of the second injection pen 300, such as the dose stop 118, and one of the zero dose stop and the further blocking part 124 of the second injection pen 300, such as the zero dose stop, are fixed to the same member of the dose delivery mechanism 354. This allows to precisely define the relative positions of the components of the dose definition mechanism 115 with respect to the components of the zero dose mechanism. The member of the dose delivery mechanism 354 exemplarily is the dose selector 28. Also the first injection pen 10 can have such a configuration.

Exemplarily, the other one of the dose stop 118 and the counter element 116 of the second injection pen 300, such as the counter element 116, and the other one of the zero dose stop and the further blocking part 124 of the second injection pen 300, such as the further blocking part 124, are fixed to the same further member of the dose delivery mechanism 354. This also allows to precisely define the relative positions of the components of the dose definition mechanism 115 with respect to the components of the zero dose mechanism. The further member can, for example, be the carrier that is rotationally movable with respect to the dose selector 28. Also the first injection pen 10 can have such a configuration.

Exemplarily, the second injection pen 300, one of the maximum dose stop 126 and the blocking part 124 of the maximum dose mechanism and one of the zero dose stop and the further blocking part 124 of the zero dose mechanism, such as the maximum dose stop 126 and the zero dose stop, are fixed to the same member of the mechanism. Furthermore, the other one of the maximum dose stop 126 and the blocking part 124 of the maximum dose mechanism and the other one of the zero dose stop and the further blocking part 124 of the zero dose mechanism, such as the blocking part 124 and the further blocking part 124, are fixed to the same further member of the mechanism. This provides for a precise alignment between the components of the maximum dose mechanism and the components of the zero dose mechanism. Also the first injection pen 10 can have such a configuration.

Irrespective of whether they are fixed to the same member of the mechanism or not, the maximum dose stop 126 and/or the minimum dose stop can be integrally formed with the member they are fixed to. Analogously, the blocking part 124 and/or the further blocking part 124 can be integrally formed with the member they are fixed to.

Exemplarily, with the second injection pen 300, the blocking part 124 of the maximum dose mechanism forms the further blocking part 124 of the minimum dose mechanism. This allows to precisely define the distance between the maximum dose position and the zero dose position. Also the first injection pen 10 can have such a configuration.

In general, the dose delivery mechanisms 54, 354, 554, 754 can comprise a clutch mechanism 113 having a first engaging part and a second engaging part, wherein the clutch mechanism 113 is closed during one of dose setting and dose delivery and opened during the other one of dose setting and dose delivery. The clutch mechanism 113 is in the opened state when the first engaging part and the second engaging part do not engage with each other and the clutch mechanism 113 is in the closed state when the first engaging part and the second engaging part engage with each other.

Exemplarily, the dose delivery mechanisms 54, 354 of the first and second injection pen comprise a clutch mechanism 113, wherein the clutch mechanism 113 rotationally locks the nut 38 to the piston rod 44 during dose delivery and rotationally releases the nut 38 from the piston rod 44 during dose setting. By rotationally locking the nut 38 to the piston rod 44, the clutch mechanism 113 forces simultaneous proximal movement of the nut 38 together with the piston rod 44. Exemplarily, the clutch mechanism 113 rotationally locks the threaded connection 189 that couples the nut 38 to the piston rod 44. The clutch mechanism 113 locks the nut 38 to the piston rod 44 in a closed state and rotationally release the nut 38 from the piston rod 44 in an opened state.

The clutch mechanism 113 exemplarily comprises a first engaging part and a second engaging part and the first and second engaging parts are configured to engage with each other to rotationally lock the nut 38 to the piston rod 44. The first engaging part and the second engaging part exemplarily are configured to become disengaged from each other by relative axial movement with respect to each other. The first engaging part is exemplarily formed by the teeth 114 of the connector 26 and the second engaging part is exemplarily formed by the teeth 108 of the dose setting element 22.

The clutch mechanism 113 exemplarily is configured to be transferred from the opened state into the closed state upon movement of the button 18, 318 and transfer of the dose delivery mechanisms 54, 354 from the dose setting state to the dose delivery state. One of the first engaging part and the second engaging part, such as the second engaging part, is axially fixed with respect to the button 18, 318 and the other one of the first engaging part and the second engaging part, such as the first engaging part, is axially fixed with respect to the housing 32, 332. Additionally or alternatively, the first engaging part is axially fixed with respect to the dosing member 23, 332.

Exemplarily, the first engaging part is rotationally fixed to the housing 32, 332 and the second engaging part is rotationally fixed to the nut 38.

The second engaging part exemplarily is rotationally fixed to the button 18, 318 and/or the dose setting element 22.

Exemplarily, the clutch mechanism 113 rotationally fixes the nut 38 to the piston rod 44 during dose delivery via the housing 32, 332 and, for example, via the dose setting element 22 and/or the button 18, 318.

Exemplarily, the clutch mechanism 113 acts between the button 1, 318 and the housing 32, 332 and/or between the dose setting element 22 and the housing 32, 332.

Exemplarily the button 18, 318 is rotationally coupled, such as permanently rotationally coupled, to one of the first engaging part and the second engaging part. As an example, the button 18, 318 constitutes the one of the first engaging part and the second engaging part.

Exemplarily, the dose delivery mechanisms 54, 354 comprise a further clutch mechanism 107, wherein the further clutch mechanism 107 rotationally locks the dose setting element 22 to one end of the spring 40 during dose setting and decouples the dose setting element 22 from the one end of the spring 40 during dose delivery. Furthermore, the further clutch mechanism 107 has a further first engaging part and a further second engaging part, wherein the further first engaging part is configured to move into engagement with the further second engaging part to rotationally lock the dose setting element 22 to the one end of the spring 40. The further clutch mechanism 107 allows to tension the spring 40 during dose setting and at the same time prevents the dose setting element 22 from rotating during dose delivery when the spring 40 relaxes again. The further first engaging part is exemplarily formed by the teeth 110 at the snap element 23 and the further second engaging part is exemplarily formed by the teeth 108 at the button 22.

The further clutch mechanism 107 exemplarily is transferred from a closed state, in which the further first engaging part engages with the further second engaging part, into an opened state, in which the further first engaging part is disengaged from the further second engaging part, by movement of the button 18, 318. The movement of the button 18, 318 exemplarily is the movement that transfers the dose delivery mechanisms 54, 354 from the dose setting state into the dose delivery state.

Exemplarily, one of the further first engaging part and the further second engaging part, such as the further second engaging part, is rotationally and axially fixed to the dose setting element 22. This allows to open and close the further clutch mechanism 107 by relative movement of the dose setting element 22 with respect to the other one of the further first engaging part and the further second engaging part.

Exemplarily, the one of the further first engaging part and the further second engaging part is rotationally and axially fixed to the button 18, 318. The dose setting element 22 thereby is at least rotationally fixed to the button 18, 318.

Exemplarily, the further clutch mechanism 107 acts between the dosing member 23, 332 and the dose setting element 22. The dosing member 23, 332 then can rotationally couple the dose setting element 22 to the one end of the spring 40.

Exemplarily, one of the further first engaging part and the further second engaging part, such as the further first engaging part, is rotationally fixed to the dosing member 23, 332. Exemplarily, the one of the further first engaging part and the further second engaging part can be axially fixed to the dosing member 23, 332.

Exemplarily, the clutch mechanism 113 comprises the first engaging part that engages the second engaging part to rotationally fix the nut 38 to the piston rod 44 during dose delivery, wherein the further second engaging part of the further clutch mechanism 107 forms the first engaging part of the clutch mechanism 113. This provides a compact construction of the clutch mechanisms 107, 113.

The nut 38 exemplarily is rotationally fixed with respect to the button 18, 318 and/or the dose setting element 22. As an example, the nut 38 is rotationally fixed and axially movable with respect to the button 18, 318 and/or the dose setting element 22. It is coupled to the button 18, 318 and/or the dose setting element 22 by a rotation lock. The rotation lock is formed by the nut 38 and one of the button 18, 318 and the dose setting element 22, such as the button 18, 318.

The nut 38 is rotationally movable with respect to the piston rod 44 when the dose delivery mechanisms 54, 354 are in the dose setting state and the nut 38 is rotationally fixed with respect to the piston rod 44 when the dose delivery mechanisms 54, 354 are in the dose delivery state. Rotation of the nut 38 with respect to the piston rod 44 during dose setting leads to axial movement due to the threaded connection between the nut 38 and the piston rod 44. By rotationally locking the nut 38 to the piston rod 44 during dose delivery, the threaded connection between the nut 38 and the piston rod 44 is blocked and the nut 38 and the piston rod 44 become axially fixed with respect to each other.

The nut 38 is turned by the dose setting element 22 during dose setting and performs an axial movement due to the threaded connection to the piston rod 44. The rotation of the nut 38 causes the nut 38 to translate axially in the distal direction along the thread located on the piston rod 44 during dose setting and to translate in the proximal direction during dose cancellation. Axial movement of the nut 38 with respect to the piston rod 44 defines the axial movement of the piston rod 44 during dose delivery and thus the amount of medicament expelled during dose delivery.

Some general remarks concerning the disclosure:

The second threaded connection 170, 172 can be provided between the driver 36 and the piston rod guide 42. However, there are multiple other options where the second threaded connection 170, 172 could be provided. For example, the second threaded connection could be provided between the dosing element 34 and the housing 32. The second threaded connection 170, 172 preferably acts between any part that is rigidly connected to the driver 36 and any part that is rigidly connected to the housing 32.

Preferably, the actuation member 18, 20, 22 is coupled via the nut 38 to the piston rod 44 in a way that an axial movement of the actuation member 18, 20, 22 during dose de-livery causes an axial movement of the nut 38 and the piston rod 44. To apply an axial force from the nut 38 to the piston rod 44 to move the piston rod 44 in the axial direction via the nut 38 during dose delivery, the nut 38 forms a first threaded connection with the piston rod 44 via the outer thread 190 meshing with the inner thread 192. The nut 38 and the piston rod 44 are blocked from relative rotational movement during dose delivery so that an axial movement of the nut 38 causes an axial movement of the piston rod 44.

Regarding the optional feature that “all parts that are configured to rotate relative to the housing 32 during dose delivery, are connected to the housing 32 via exactly one threaded connection 170, 172”, it is pointed out that the dose delivery mechanism 54 can comprise a driver 36, a dose sleeve 34 and/or a snap element 24. One or more of these parts can be configured to rotate relative to the housing 32 during dose delivery. Furthermore, the delivery mechanism 54 can have one or more further parts that are configured to rotate relative to the housing 32 during dose delivery. All parts of the delivery mechanism 54 that are configured to rotate relative to the housing 32 during dose delivery can form a sub-assembly. This sub-assembly can form exactly one threaded connection 170, 172 with the housing 32 or any part rigidly connected to the housing 32.

The coupling means 100, 102 can be formed on the injection button and the snap element 24. However, the coupling means 100 can alternatively be formed on the snap ring 20 or the dose setting knob 22. The coupling means 102 could generally be formed on any part that is not axially movable relative to the housing. The person skilled in the art understands that there are multiple options where to place the coupling means 100, 102. If the coupling means 100, 102 are configured to permanently axially lock the dose setting knob 22 to the dose setting device or the housing 50, this can be done by multiple ways, e.g. by a permanent form fitted connection or by a permanent friction fitted connection.

Generally, the dose setting mechanism can comprise a clutch that connects a nut 38 to a piston rod 44, with the nut 38 being rotationally fixed to the piston rod 44 during dispensing and rotational relative to the piston rod 44 during dose setting. Preferably, rotating the dose setting knob effects rotation of the nut 38 relative to the piston rod 44 during dose setting. There are multiple ways how to design a connection between the dose setting knob and the nut 38.

Generally, the piston rod 44 can be rotationally fixed with respect to the housing during dose delivery/dispensing.

The proximal end 14 comprises a dispensing outlet. The distal end 12 is arranged at an opposite end of the proximal end 14. The dispensing outlet can comprise one of a needle, a cannula, and a point of connection for a needle or cannula.

The cover 16 can cover the part of the dose delivery activation mechanism configured to activate dose delivery. The dose delivery activation mechanism are the parts that are configured to permit a dose dispensing from the delivery device. The activation mechanism can comprise the injection button 18 forming a distal end face of the injection pen. The cover 16 can also cover the dose setting knob 22. Thereby, the cover 16 prevents setting the dose and/or activating the injection before a mixing of the two components is done.

The as-delivered condition is a condition in which all parts of the dose delivery mechanism 54 are arranged at the same relative position with respect to one another as they are following manufacture of the dose delivery mechanism 54. In other words, the as-delivered condition is a condition in which the user has not rotated or otherwise changed the position of the dose setting member 34 relative to the housing. Hence, when a dose has been set by the user, the dose delivery mechanism 54 is not in an “as-delivered condition” anymore but instead in a “set state”. According to an embodiment, in the “as-delivered condition”, the dose setting knob 22 is not rotatable towards a zero-dose position. According to an embodiment, the actuation is blocked in the “as-delivered condition” to prevent accidental activation.

The dose delivery mechanism can comprise the activation member 18, the dose setting knob 22, the snap element 24, the dose selector 28 and/or the clip 30. The activation member 18 can be configured to be pushed by the user to start the injection process. The snap element 24 is preferably configured to engage with the dose selector 28, preferably in different rotational positions, to define a settable dose. The clip 30 can be used in combination with the knob cover 16 to avoid movement of the activation member 18 relative to the housing 32 when the pen drops onto its proximal end 14.

The present disclosure also relates to the following embodiments:
1. A dose delivery mechanism (54, 354, 554, 754) for a medicament delivery device (10, 300, 500, 700) comprising:
a housing (32, 332, 532);
a piston rod (44); and
an adjusting element (18, 318, 518),
wherein the housing (32, 332, 532) is configured to connect to a medicament container (48, 348) sealed by a plunger (210),
wherein the dose delivery mechanism (54, 354, 554, 754) has a preassembled state and an assembled state,
wherein, in the assembled state, the dose delivery mechanism (54, 354, 554, 754) is configured to move the piston rod (44) axially in a proximal direction (1) with respect to the housing (32, 332, 532) during dose delivery such that the piston rod (44) exerts an axial force in the proximal direction (1) on the plunger (210) of the medicament container (48, 348) to expel a medicament from the medicament container (48, 348), and
wherein, in the preassembled state, the adjusting element (18, 318, 518) is configured to perform a rotation with respect to the housing (32, 332, 532),
wherein the rotation of the adjusting element (18, 318, 518) causes an axial movement of the piston rod (44) for adjusting an axial position of the piston rod (44) with respect to the housing (32, 332, 532) prior to transfer of the dose delivery mechanism (54, 354, 554, 754) from the preassembled state into the assembled state.
2. The dose delivery mechanism (54, 354, 554, 754) of embodiment 1,
wherein the dose delivery mechanism (54, 354, 554, 754) is assembled from the same components in the preassembled state and in the assembled state.
3. The dose delivery mechanism (54, 354, 554, 754) of at least one of the preceding embodiments,
wherein the transfer of the dose delivery mechanism (54, 354, 554, 754) from the preassembled state into the assembled state does not comprise an addition and/or removal of a component of the dose delivery mechanism (54, 354, 554, 754).
4. The dose delivery mechanism (54, 354, 554, 754) of at least one of the preceding claims,
wherein all movable components of the dose delivery mechanism (54, 354, 554, 754) except for the adjusting element (18, 218, 518), the piston rod (44) and components axially fixed to the piston rod (44) are positioned in a respective zero dose position with respect to the housing (32, 332, 532) in the preassembled state,
wherein the movable components are configured to reach the respective zero dose position at the end of dose delivery and/or prior to setting a dose with the dose delivery mechanism (54, 354, 554, 754) in the assembled state,
wherein, for example, each zero dose position is an axial position with respect to the housing.
5. The dose delivery mechanism (54, 354, 554, 754) of at least one of the preceding claims,
wherein the adjusting element (18, 218, 518)
6. The dose delivery mechanism (54, 354, 554, 754) of at least one of the preceding embodiments,
wherein the adjusting element (18, 318, 518) is configured to be rotated until a bearing (46) located at the piston rod (44) contacts the plunger (210) of the medicament container (48, 348).
7. The dose delivery mechanism (54, 354, 554, 754) of at least one of the preceding embodiments,
wherein an outer rim (19) of the adjusting element (18, 318, 518) is accessible to an assembler of the device in the preassembled state to effect rotation of the adjusting element (18, 318, 518) and axial movement of the piston rod (44),
wherein, for example, the adjusting element (18, 318, 518) is configured to cause proximal movement of the piston rod (44) when being turned by the assembler.
8. The dose delivery mechanism (54, 354, 554, 754) of at least one of the preceding embodiments,
wherein an outer rim (19) of the adjusting element (18, 318, 518) is located at a distal end of the dose delivery mechanism (54, 354, 554, 754).
9. The dose delivery mechanism (54, 354, 554, 754) of at least one of the preceding embodiments,
wherein an outer rim (19) of the adjusting element (18, 318, 518) is laterally accessible in a radial direction perpendicular to a longitudinal axis of the dose delivery mechanism (54, 354, 554, 754) and/or
wherein the outer rim (19) is not covered by other members of the dose delivery mechanism, such as the housing (32, 332, 532), in the radial direction, such as in all radial directions.
10. The dose delivery mechanism (54, 354, 554, 754) of at least one of the preceding embodiments,
wherein the adjusting element (18, 318, 518) protrudes in a distal direction from the housing (32, 332, 532) in the preassembled state,
wherein, for example, the adjusting element (18, 318, 518) protrudes in a distal direction from the remaining members of the dose delivery mechanism (54, 354, 554, 754) in the preassembled state.
11. The dose delivery mechanism (54, 354, 554, 754) of at least one of the preceding embodiments,
wherein the adjusting element (18, 318, 518) is in a preassembled position with respect to the housing (32, 332, 532) in the preassembled state,
wherein the adjusting element (18, 318, 518) is configured to be transferred from the preassembled position into an assembled position with respect to the housing (32, 332, 532) when transferring the dose delivery mechanism (54, 354, 554, 754) from the preassembled state into the assembled state,
wherein, for example, the transfer of the adjusting element (18, 318, 518) causes a transfer of the dose delivery mechanism (54, 354, 554, 754) from the preassembled state into the assembled state.
12. The dose delivery mechanism (54, 354, 554, 754) of embodiment 11,
wherein the adjusting element (18, 318, 518) is configured to perform the rotation when being located in the preassembled position.
13. The dose delivery mechanism (754) of embodiment 11,
wherein the adjusting element (518) is configured to perform the rotation when being located in an adjusting position that deviates from the preassembled position,
wherein, for example, the adjusting element (518) is configured to be pushed in the proximal direction (1) from the preassembled position into the adjusting position.
14. The dose delivery mechanism (54, 354, 554, 754) of at least one of embodiments 11 to 13,
wherein the dose delivery mechanism (54, 354, 554, 754) is configured to hold the adjusting element (18, 318, 518) in the preassembled position with respect to the housing (32, 332, 532) in the preassembled state.
15. The dose delivery mechanism (354, 554, 754) of at least one of embodiments 11 to 14,
wherein the dose delivery mechanism (354, 554, 754) comprises a biasing element (250) that biases the adjusting element (318, 518) into the preassembled position with respect to the housing (332, 532) in the preassembled state.
16. The dose delivery mechanism (54, 354, 554, 754) of at least one of embodiments 11 to 15,
wherein the adjusting element (18, 318, 518) is configured to move axially, for example in the proximal direction (1), from the preassembled position into the assembled position upon transferring the dose delivery mechanism (54, 354, 554, 754) from the preassembled state into the assembled state.
17. The dose delivery mechanism (54, 354, 554, 754) of at least one of embodiments 11 to 16,
wherein the adjusting element (18, 318, 518) is blocked, such as irreversibly blocked, in the assembled position from returning into the preassembled position with respect to the housing (32, 332, 532).
18. The dose delivery mechanism (54, 354, 554, 754) of embodiment 17,
wherein the dose delivery mechanism (54, 354, 554, 754) comprises a latching mechanism (97, 597) that is configured to prevent the adjusting element (18, 318, 518) from moving from the assembled position into the preassembled position.
19. The dose delivery mechanism (54, 354, 554, 754) of embodiment 18,
wherein the dose delivery mechanism (54, 354, 554, 754) comprises a counter member (23, 323, 528, 720),
wherein the latching mechanism (97, 597) comprises a latch part (98a, 600) of the adjusting element (18, 318, 518) and a latch counterpart (102, 529) of the counter member (23, 323, 528, 720),
wherein the latch part (98a, 600) and the latch counterpart (102, 529) are configured to directly engage with each other in the assembled state to block movement of the adjusting element (18, 318, 518) with respect to the housing (32, 332, 532).
20. The dose delivery mechanism (54, 354, 554, 754) of at least one of the preceding embodiments,
wherein the dose delivery mechanism (54, 354, 554, 754) comprises a further latching mechanism (99, 599) that is configured to prevent detachment of the adjusting element (18, 318, 518) from the housing (32, 332, 532) in the preassembled state.
21. The dose delivery mechanism (54, 354, 554, 754) of embodiment 20,
wherein the further latching mechanism (99, 599) is configured to hold the adjusting element (18, 318, 518) in a preassembled position, such as a preassembled axial position, with respect to a counter member (720) during axial movement of the adjusting element (18, 318, 518) in the preassembled state, for example, during axial movement of the adjusting element (18, 318, 518) from a preassembled position into an adjusting position in the preassembled state.
22. The dose delivery mechanism (54, 354, 554, 754) of at least one of embodiments 20 and 21,
wherein the further latching mechanism (99, 599) comprises a further latch part (98b, 600) of the adjusting element (18, 318, 518) and a further latch counterpart (102, 530) of a further counter member (23, 323, 528, 720),
wherein the further latch part (98b, 600) and the further latch counterpart (102, 530) are configured to directly engage with each other in the preassembled state to block movement of the adjusting element (18, 318, 518) with respect to the housing (32, 332, 532).
23. The dose delivery mechanism (54, 354, 554, 754) of at least one of embodiments 18 and 19 and of at least one of embodiments 20 and 21,
wherein the latching mechanism (97, 597) and the further latching mechanism (99, 599) share a single latch part (98a, 98b, 600) or a single latch counterpart (102, 529, 530).
24. The dose delivery mechanism (54, 354, 554, 754) of at least one of the preceding embodiments,
wherein the dose delivery mechanism (54, 354, 554, 754) is configured to transfer the rotation of the adjusting element (18, 318, 518) into the axial movement of the piston rod (44) via a single threaded connection (189) in the preassembled state,
wherein the threaded connection (189) comprises a first threaded element (44) that is threadedly engaged with a second threaded element (38, 523),
wherein, for example, a pitch of the single threaded connection (189) defines a ratio of an axial distance to a circumferential distance and the piston rod (44) travels the axial distance with respect to the housing (32, 332, 532) upon rotation of the adjusting element (18, 318, 518) by the circumferential distance.
25. The dose delivery mechanism (54, 354, 554, 754) of embodiment 24,
wherein the first and second threaded elements (38, 44, 523) rotate with respect to each other during one of dose setting and dose delivery in the assembled state,
wherein the first and second threaded elements (38, 44, 523) do not rotate with respect to each other during the other one of dose setting and dose delivery in the assembled state.
26. The dose delivery mechanism (54, 354, 554, 754) of at least one of embodiments 24 and 25,
wherein, during dose delivery in the assembled state, the first threaded element (44) is rotationally stationary with respect to a third element (32, 332, 532) of the dose delivery mechanism (54, 354, 554, 754) and the second threaded element (38, 523) is rotated with respect to the third element (32, 332, 532),
wherein, during adjustment of the piston rod (44) in the preassembled state, the first threaded element (44) is rotated with respect to the third element (32, 332, 532) of the dose delivery mechanism (54, 354, 554, 754) and the second threaded element (38, 523) is rotationally stationary with respect to the third element (32, 332, 532).
27. The dose delivery mechanism (54, 354, 554, 754) of embodiment 26,
wherein the third element (32, 332, 532) is the housing (32, 332, 532).
28. The dose delivery mechanism (54, 354, 554, 754) of at least one of embodiments 24 to 27,
wherein the first threaded element (44) is the piston rod (44).
29. The dose delivery mechanism (54, 354, 554, 754) of at least one of embodiments 24 to 28,
wherein one of the first threaded element (44) and the second threaded element (38, 523) retains its axial position with respect to the housing (32, 332, 532) during the axial movement of the piston rod (44) in the preassembled state.
30. The dose delivery mechanism (54, 354, 554, 754) of at least one of embodiments 24 to 29,
wherein one of the first threaded element (44) and the second threaded element (38, 523) are configured to not rotate during the rotation of the adjusting element (18, 318, 518) in the preassembled state.
31. The dose delivery mechanism (554, 754, 754) of at least one of embodiments 24 to 30,
wherein the adjusting element (518) is coupled to the threaded connection (189) via the piston rod (44).
32. The dose delivery mechanism (554, 754, 754) of at least one of embodiments 24 to 31,
wherein the threaded connection (189) acts between the piston rod (44) and the housing (532).
33. The dose delivery mechanism (554, 754, 754) of at least one of embodiments 24 to 32,
wherein the second threaded element (523) is a dosing member (523) of the dose delivery mechanism (554, 754, 754),
wherein the dosing member (523) is rotationally stationary with respect to the housing (532) during the rotation of the adjusting element (518) in the preassembled state.
34. The dose delivery mechanism (54, 354) of at least one of embodiments 24 to 30,
wherein the threaded connection (189) acts between the adjusting element (18, 318) and the piston rod (44).
35. The dose delivery mechanism (554, 754, 754) of at least one of the preceding embodiments,
wherein the piston rod (44) is threadedly coupled to, for example threadedly engaged with, a dosing member (523) of the dose delivery mechanism (554, 754, 754),
wherein the dosing member (523) is rotationally stationary with respect to the housing (532) during the rotation of the adjusting element (518) in the preassembled state.
36. The dose delivery mechanism (554, 754, 754) of at least one of the preceding embodiments,
wherein the adjusting element (518) is rotationally fixed to the piston rod (44),
wherein, in the preassembled state, the rotation of the adjusting element (18, 318) causes rotation of the piston rod (44).
37. The dose delivery mechanism (554, 754, 754) of at least one of the preceding embodiments,
wherein the piston rod (44) is threadedly coupled, such as threadedly engaged with, a dosing member (523) of the dose delivery mechanism (554, 754, 754),
wherein the dosing member (523) is rotationally movable and axially fixed with respect to the housing (532),
wherein, during dose setting in the assembled state, the piston rod (44) is rotationally fixed to the dosing member (523),
wherein, during dose delivery in the assembled state, the piston rod (44) is rotationally fixed to the housing (532) and rotatable with respect to the dosing member (532).
38. The dose delivery mechanism (554, 754, 754) of at least embodiment 37,
wherein the dosing member (523) is threadedly coupled to the housing (532).
39. The dose delivery mechanism (554, 754, 754) of at least one of embodiments 37 and 38,
wherein rotation of the adjusting element (518) during dose setting in the assembled state causes the dosing member (523) to rotate with respect to the housing (532).
40. The dose delivery mechanism (554, 754, 754) of at least one of embodiments 37 to 39,
wherein axial movement of the adjusting element (518) during dose delivery in the assembled state causes the dosing member (523) to rotate with respect to the housing (532).
41. The dose delivery mechanism (554, 754, 754) of at least one of the preceding embodiments,
wherein the dose delivery mechanism (54, 354) comprises a coupling element (520, 720) that is rotationally fixed to, for example rotationally fixedly engaged with, the piston rod (44),
wherein, in the preassembled state, the rotation of the adjusting element (18, 318) causes rotation of the coupling element (520, 720).
42. The dose delivery mechanism (554, 754, 754) of at least embodiment 41,
wherein the coupling element (520, 720) is axially fixed to the adjusting element (18, 318) and/or axially movable with respect to the piston rod (44).
43. The dose delivery mechanism (54, 354) of at least one of the preceding embodiments,
wherein the dose delivery mechanism (54, 354) comprises a nut (38) that is threadedly connected to, for example threadedly engaged with, the piston rod (44),
wherein, in the preassembled state, the rotation of the adjusting element (18, 318) causes rotation of the nut (38) to cause the piston rod (44) to move axially relative to the housing (32, 332).
44. The dose delivery mechanism (54, 354) of embodiment 35,
wherein the adjusting element (18, 318) is rotationally fixed to the nut (38) and axially slidable relative to the nut (38).
45. The dose delivery mechanism (54, 354) of at least one of embodiments 35 and 44,
wherein, in the assembled state, the nut (38) is turned by the adjusting element (18, 318) during dose setting and performs an axial movement due to the threaded connection (189) to the piston rod (44).
46. The dose delivery mechanism (54, 354) of at least one of embodiments 35 to 45,
wherein, in the assembled state, rotation of the nut (38) causes the nut (38) to translate axially in a distal direction along threads (190) located on the piston rod (44) during dose setting and to translate in the proximal direction (1) during dose cancellation.
47. The dose delivery mechanism (54, 354) of at least one of embodiments 35 to 46,
wherein, in the assembled state, the nut (38) does not rotate during dose delivery, moving only axially with the piston rod (44) a distance in the proximal direction, wherein the distance is directly proportional to a set dose.
48. The dose delivery mechanism (54, 354, 554, 754) of at least one of the preceding embodiments,
wherein the dose delivery mechanism (54, 354, 554, 754) comprises a further member (23, 323, 523),
wherein the adjusting element (18, 318, 518) is rotationally decoupled from the further member (23, 323, 523) during adjustment of the piston rod (44) in the preassembled state,
wherein the adjusting element (18, 318, 518) is rotationally coupled to the further member (23, 323, 523) during dose setting in the assembled state,
wherein the adjusting element (18, 318, 518) is rotationally decoupled from the further member (23, 323, 523) during dose delivery in the assembled state.
49. The dose delivery mechanism (54, 354, 554, 754) of embodiment 48,
wherein the further member (23, 323, 523) is threadedly connected to the housing (32, 332, 532).
50. The dose delivery mechanism (54, 354, 554, 754) of at least one of embodiments 48 and 49,
wherein the further member (23, 323, 523) is a dose indication member indicating a set dose.
51. The dose delivery mechanism (54, 354) of at least one of embodiments 48 to 50,
wherein the further member (23, 323) rotates with respect to the piston rod (44) during dose setting and/or
wherein the further member (23, 323) does not rotate with respect to the piston rod (44) during the adjustment of the position of the piston rod (44) in the preassembled state.
52. The dose delivery mechanism (54, 354, 554, 754) of at least one of embodiments 48 to 51,
wherein the further member (23, 323, 523) maintains its axial position with respect to the housing (32, 332, 532) of the dose delivery mechanism (54, 354, 554, 754) upon the rotation of the adjusting element (18, 318, 518) in the preassembled state.
53. The dose delivery mechanism (54, 354, 554, 754) of at least one of the preceding embodiments,
wherein the adjusting element (18, 318, 518) is rotationally decoupled from an additional member (32, 332, 532) of the dose delivery mechanism (54, 354, 554, 754) during adjustment of the piston rod (44) in the preassembled state,
wherein the adjusting element (18, 318, 518) is rotationally decoupled from the additional member (32, 332, 532) during dose setting in the assembled state,
wherein the adjusting element (18, 318, 518) is rotationally fixed to the additional member (32, 332, 532) during dose delivery in the assembled state.
54. The dose delivery mechanism (54, 354, 554, 754) of embodiment 53,
wherein the additional member (32, 332, 532) is the housing (32, 332, 532).
55. The dose delivery mechanism (54, 354) of at least one of the preceding embodiments,
wherein the adjusting element (18, 318) is rotatable with respect to a counter element (22) in the preassembled state and rotationally fixed, such as irreversibly rotationally fixed, to the counter element (22) in the assembled state.
56. The dose delivery mechanism (54, 354) of embodiment 55,
wherein the adjusting element (18, 318) is in a first axial position with respect to the counter element (22) of the dose delivery mechanism (54, 354) in the preassembled state,
wherein the adjusting element (18, 318) is configured to move axially from the first axial position into a second axial position with respect to the counter element (22) upon transfer of the dose delivery mechanism (54, 354) from the preassembled state into the assembled state,
wherein the adjusting element (18, 318) is axially fixed, such as irreversibly axially fixed, to the counter element (22) in the assembled state.
57. The dose delivery mechanism (54,354) of embodiment 56,
wherein the dose delivery mechanism (54, 354) comprises a latching mechanism that acts between the adjusting element (18, 318) and the counter element (22),
wherein the latching mechanism is configured to block movement of the adjusting element (18, 318) from the second position into the first position in the assembled state.
58. The dose delivery mechanism (54, 354) of at least one of embodiments 55 to 57,
wherein the counter element (22) is a dose setting element (22) of the dose delivery mechanism (54, 354),
wherein the dose setting element (22) is configured to be gripped by the user of the dose delivery mechanism (54, 354) in the assembled state to set a dose to be delivered.
59. The dose delivery mechanism (54, 354) of at least one of embodiments 55 to 58,
wherein the adjusting element (18,318) protrudes distally from the counter element (22) in the preassembled state.
60. The dose delivery mechanism (54, 354) of at least one of embodiments 55 to 59,
wherein the adjusting element (18, 318) does not protrude distally from the counter element (22) in the assembled state.
61. The dose delivery mechanism (54, 354) of embodiment 55 to 60,
wherein the dose delivery mechanism (54, 354) comprises a rotational lock (89), wherein the rotational lock (89) allows rotational movement between the adjusting element (18, 318) and the counter element (22) in the preassembled state of the dose delivery mechanism (54, 354),
wherein the adjusting element (18, 318) is rotationally fixed with respect to the counter element (22) in the assembled state via the rotational lock (89),
wherein the rotational lock (89) allows fixation of the adjusting element (18, 318) to the counter element (22) in a multitude of mutual relative rotational positions.
62. The dose delivery mechanism (54, 354) of embodiment 61,
wherein the rotational lock (89) comprises a toothed part (93) defining the multitude of rotational positions and an engaging part (90) that is configured to engage with the toothed part (93) upon transfer of the dose delivery mechanism (54, 354) from the preassembled state into the assembled state to rotationally lock the adjusting element (18, 318) to the counter element (22).
63. The dose delivery mechanism (54, 354) of at least one of embodiments 55 to 62,
wherein the dose delivery mechanism (54, 354) comprises an axial lock (81),
wherein the axial lock (81) allows axial movement between the adjusting element (18) and the counter element (22) in the preassembled state of the dose delivery mechanism (54, 354),
wherein axial lock (81) prevents axial movement between the adjusting element (18, 318) and the counter element (22) in the assembled state.
64. The dose delivery mechanism (54, 354) of embodiment 63,
wherein the axial lock (81) allows axial fixation of the adjusting element (18, 318) to the counter element (22) in a multitude of mutual relative rotational positions.
65. The dose delivery mechanism (54, 354) of at least one of embodiments 55 to 64,
wherein the dose delivery mechanism (54, 354) comprises a connector (20),
wherein the adjusting element (18) is rotationally and/or axially fixed to the counter element (22) in the assembled state via the connector (20).
66. The dose delivery mechanism (54, 354) of embodiment 65,
wherein the connector (20) is axially locked to the counter element (22) both in the preassembled state and in the assembled state.
67. The dose delivery mechanism (54, 354) of at least one of embodiments 65 and 66,
wherein the connector (20) is rotationally locked to the counter element (22) both in the preassembled state and in the assembled state.
68. The dose delivery mechanism (54, 354) of at least one of embodiments 61 and 62 and at least one of embodiments 65 to 67,
wherein the rotational lock (89) is located between the adjusting element (18, 318) and the connector (20).
69. The dose delivery mechanism (54, 354) of embodiments 62 and 68,
wherein the connector (20) comprises one of the toothed part (93) and the engaging part (90) and
wherein the adjusting element (18, 318) comprises the other one of the toothed part (93) and the engaging part (90).
70. The dose delivery mechanism (54, 354) of at least one of embodiments 63 and 64 and at least one of embodiments 65 to 69,
wherein the axial lock (81) is located between the adjusting element (18) and the connector (20).
71. The dose delivery mechanism (54, 354) of at least one of the preceding embodiments,
wherein the adjusting element (18, 318) is configured to rotate with respect to the piston rod (44) upon the rotation with respect to the housing (32) in the preassembled state.
72. The dose delivery mechanism (54, 354) of at least one of the preceding embodiments,
wherein the piston rod (44) is rotationally fixed with respect to the housing (32, 332) in the preassembled state.
73. The dose delivery mechanism (554, 754) of at least one of embodiments 1 to 70,
wherein the adjusting element (518) is rotationally fixed with respect to the piston rod (44) in the preassembled state.
74. The dose delivery mechanism (54, 354, 554, 754) of at least one of the preceding embodiments,
wherein the piston rod (44) is rotationally fixed with respect to the housing (32, 332, 532) in the assembled state during dose setting and/or during dose delivery.
75. The dose delivery mechanism (54) of at least one of the preceding embodiments,
wherein the dose delivery mechanism (54) comprises a clutch mechanism (107, 113) comprising a first clutch member (22) and a second clutch member (23, 26),
wherein, in the assembled state, the clutch mechanism (107, 113) is closed during one of dose setting and dose delivery and opened during the other one of dose setting and dose delivery,
wherein the adjusting element (18) is rotationally decoupled from both the first clutch member (22) and the second clutch member (23, 26) in the preassembled state.
76. The dose delivery mechanism (54) of embodiment 75,
wherein the adjusting element (18) is rotationally coupled, such as permanently rotationally coupled, to one of the first clutch member (22) and the second clutch member (23, 26) in the assembled state.
77. The dose delivery mechanism (54, 354, 554, 754) of at least one of the preceding embodiments,
wherein the adjusting element (18, 318, 518) is in a first axial position with respect to a retaining member (28, 528, 720) of the dose delivery mechanism (54, 354, 554, 754) in the preassembled state,
wherein the adjusting element (18, 318, 518) is configured to move axially from the first axial position into a second axial position with respect to the retaining member (28, 528, 720) upon transfer of the dose delivery mechanism (54, 354, 554, 754) from the preassembled state into the assembled state.
78. The dose delivery mechanism (754) of embodiment 77,
wherein the adjusting element (18, 318, 518) is rotationally fixed to the retaining member (28, 528, 720) during dose delivery in the assembled state.
79. The dose delivery mechanism (754) of at least one of embodiments 77 and 78,
wherein the adjusting element (518) is rotationally fixed with respect to the retaining member (720) in the preassembled state.
80. The dose delivery mechanism (754) of at least one of embodiments 77 to 79,
wherein the retaining member (720) is formed by a coupling element (720) that rotationally fixes the adjusting element (518) to the piston rod (44).
81. The dose delivery mechanism (754) of at least one of embodiments 77 to 80,
wherein the retaining member (720) is configured to be moved axially, for example in the proximal direction, together with the adjusting element (518) into an adjusting position with respect to the housing (32, 332, 532),
wherein the adjusting element (518) is configured to perform the rotation with respect to the housing (32, 332, 532) in the adjusting position to cause the axial movement of the piston rod (44).
82. The dose delivery mechanism (754) of at least embodiment 81,
wherein axial movement of the retaining member (720) opens a clutch mechanism (507) to rotationally decouple a further member (523) from the adjusting element (518) during the rotation with respect to the housing (32, 332, 532) in the adjusting position,
wherein, for example, the further member (523) is threadedly coupled to the piston rod (44) and/or axially fixed with respect to the housing (32, 332, 532),
wherein, for example, the further member (523) is formed by a dosing member (523) of the dose delivery mechanism (754).
83. The dose delivery mechanism (754) of at least embodiment 82,
wherein the adjusting element (518) is configured to be moved axially, for example in the proximal direction, with respect to the retaining member (720) in the adjusting position to transfer the dose delivery mechanism (754) from the preassembled state into the assembled state.
84. The dose delivery mechanism (754) of at least one of embodiments 77 to 83,
wherein the adjusting element (518) is axially coupled to the retaining member (720) via a latching mechanism (599).
85. The dose delivery mechanism (754) of at least one of embodiments 81 to 83 and embodiment 84,
wherein the latching mechanism (599) is configured to transfer axial movement of the adjusting element (518) into axial movement of the retaining member (720) during movement of the adjusting element (518) into the adjusting position.
86. The dose delivery mechanism (754) of at least embodiment 85,
wherein the latching mechanism (599) is configured to allow proximal movement of the adjusting element (518) with respect to the retaining member (720) in the adjusting position to transfer the dose delivery mechanism (754) from the preassembled state into the assembled state.
87. The dose delivery mechanism (54, 354, 554, 754) of embodiment 78,
wherein the adjusting element (18, 318, 518) is rotatable with respect to the retaining member (28, 528) in the preassembled state.
88. The dose delivery mechanism (54, 354, 554) of embodiment 87,
wherein the adjusting element (18, 318, 518) is rotatable with respect to the retaining member (28, 528) during dose setting in the assembled state.
89. The dose delivery mechanism (554) of at least one of embodiments 87 and 88,
wherein the adjusting element (518) is axially movable with respect to the retaining member (528) in the assembled state.
90. The dose delivery mechanism (554, 754) of at least one of the preceding embodiments,
wherein the dose delivery mechanism (554, 754) comprises a clutch mechanism (507) having a first clutch member (520, 720) and a second clutch member (524),
wherein the clutch mechanism (507) connects the adjusting element (518) to a further member (523),
wherein the clutch mechanism (507) is opened in the preassembled state during the rotation of the adjusting element (518) thus allowing a rotation of the adjusting element (518) with respect to the further member (523),
wherein the clutch mechanism (507) is opened in the assembled state during dose delivery thus allowing a rotation of the adjusting element (518) with respect to the further member (523).
91. The dose delivery mechanism (554, 754) of embodiment 90,
wherein the clutch mechanism (507) is closed in the assembled state during dose setting.
92. The dose delivery mechanism (554, 754) of at least one of embodiments 88 and 91,
wherein the clutch mechanism (507) is biased into a closed configuration in the preassembled state,
wherein the adjusting element (518) is configured to be axially moved with respect to the housing (532), for example in the proximal direction (1), in the preassembled state to open the clutch mechanism (507).
93. The dose delivery mechanism (554) of at least one of embodiments 90 and 91,
wherein the dose delivery mechanism (554) is configured to hold the clutch mechanism (507) in the opened state in the preassembled state.
94. The dose delivery mechanism (554) of at least one of embodiments 90 to 91 and 93,
wherein the clutch mechanism (507) is only allowed to close from the opened state in the preassembled state when transferring the dose delivery mechanism (554, 754) from the preassembled state into the assembled state.
95. The dose delivery mechanism (554, 754) of at least one of embodiments 90 to 91, 93 and 94,
wherein the clutch mechanism (507) comprises a first clutch part (508) and a second clutch part (509),
wherein the first clutch part (508) and the second clutch part (509) are engaged with each other in the closed state of the clutch mechanism (507) and disengaged from each other in the opened state of the clutch mechanism (507),
wherein the first clutch part (508) is located at a first axial side from the second clutch part (509) in the opened state in the preassembled state of the dose delivery mechanism (554),
wherein the first clutch part (508) is located at a second axial side from the second clutch part (509) in the opened state in the assembled state of the dose delivery mechanism (54),
wherein the second axial side is opposite the first axial side.
96. The dose delivery mechanism (354, 554, 754) of at least one of the preceding embodiments,
wherein the adjusting element (318, 518) is biased in a distal direction when the dose delivery mechanism (354, 554, 754) is in the preassembled state.
97. The dose delivery mechanism (54, 354, 554, 754) of at least one of the preceding embodiments,
wherein the adjusting element (18, 318, 518) is configured to take up a dose setting position in the assembled state,
wherein the adjusting element (18, 318, 518) is movable, for example axially movable, in the assembled state to leave the dose setting position.
98. The dose delivery mechanism (54, 354, 554, 754) of embodiment 97,
wherein the adjusting element (18, 318, 518) is biased into the dose setting position in the assembled state.
99. The dose delivery mechanism (54, 354, 554, 754) of at least one of embodiments 97 and 98,
wherein the adjusting element (18, 318, 518) is configured to move proximally from the dose setting position into a dose delivery position in the assembled state.
100. The dose delivery mechanism (54, 354, 554, 754) of embodiment 99,
wherein the dose delivery mechanism (54, 354, 554, 754) comprises a clutch mechanism (107, 507) and an actuation element (18, 318, 520),
wherein the clutch mechanism (107, 507) is transferred from a closed state into an opened state upon proximal movement of the adjusting element (18, 318, 518) from the dose setting position into the dose delivery position to effect proximal movement of the piston rod (44) upon proximal movement of the actuation element (18, 318, 520).
101. The dose delivery mechanism (554, 754) of at least one of the preceding embodiments,
wherein the adjusting element (518) is configured as a dose setting element of the dose delivery mechanism (554, 754),
wherein the dose setting element is configured to be gripped by the user of the dose delivery mechanism (554, 754) to set a dose to be delivered in the assembled state.
102. The dose delivery mechanism (554, 754) of at least embodiment 101,
wherein the adjusting element (518) comprises a gripping surface and/or a radially outermost surface that is accessible to the user for setting the dose to be delivered in the assembled state,
wherein the adjusting element (518) completely comprises the gripping surface and/or the radially outermost surface.
103. The dose delivery mechanism (54, 354, 554, 754) of at least one of the preceding claims,
wherein a distal end surface of the dose delivery mechanism (54, 354, 554, 754) forms part of the adjusting element (518) or is rigidly connected with the adjusting element (518). 104 The dose delivery mechanism (554, 754) of at least one of the preceding claims,
wherein the adjusting element (518) forms at least part of a knob that is placed at the distal end of the dose delivery mechanism (554, 754).
105. The dose delivery mechanism (54, 354, 554, 754) of at least one of the preceding embodiments,
wherein the adjusting element (18, 318, 518) is configured to rotate in the assembled state to set a dose of the medicament to be delivered by the dose delivery mechanism (54, 354, 554, 754).
106. The dose delivery mechanism (54, 354, 554, 754) of at least one of the preceding embodiments,
wherein the dose delivery mechanism (54, 354, 554, 754) comprises a locking mechanism (113, 513),
wherein the locking mechanism (113, 513) rotationally locks the adjusting element (18, 318, 518) to the housing (32, 332, 532) during dose delivery in the assembled state.
107. The dose delivery mechanism (54, 354, 554, 754) of at least one of the preceding embodiments,
wherein the dose delivery mechanism (54, 354, 554, 754) comprises a dose definition mechanism (115) that defines doses settable by a user in the assembled state,
wherein the dose definition mechanism (115) defines rotational positions of the adjusting element (18, 318, 518) with respect to the housing (32, 332, 532) that correspond to the doses settable by the user.
108. The dose delivery mechanism (54, 354, 554, 754) of embodiment 107,
wherein the dose definition mechanism (115) comprises at least one engagement feature (116) and at least one dose stop (118) that rotate with respect to each other upon rotation of the adjusting element (18, 318, 518) in the assembled state and that engage with each other upon setting a dose in the assembled state.
109. The dose delivery mechanism (754) of at least one of embodiments 107 and 108,
wherein the dose definition mechanism is active during the rotation of the adjusting element (518) in the preassembled state.
110. The dose delivery mechanism (54, 354, 554, 754) of at least one of embodiments 107 and 108,
wherein the dose definition mechanism is not active during the rotation of the adjusting element (18, 318, 518) in the preassembled state.
111. The dose delivery mechanism (54, 354) of at least embodiments 108 and 110,
wherein the engagement feature (116) and the at least one dose stop (118) do not rotate with respect to each other during the rotation of the adjusting element (18, 318, 518) in the preassembled state.
112. The dose delivery mechanism (554) of at least embodiments 108 and 110,
wherein the engagement feature (116) and the at least one dose stop (118) rotate with respect to each other during the rotation of the adjusting element (18, 318, 518) in the preassembled state,
wherein the engagement feature (116) and the at least one dose stop (118) do not engage with each other in the preassembled state,
wherein, for example, the engagement feature (116) and the at least one dose stop (118) are located axially offset from each other in the preassembled state.
113. The dose delivery mechanism (54) of at least one of the preceding embodiments,
wherein the housing (32) comprises a connector (43) for connecting the medicament container (48) to the housing (32),
wherein the connector (43) is configured to connect the medicament container (48) axially movable to the housing (32) so that the medicament container (48) is configured to perform an axial movement from a receiving position into an operating position after connection to the housing (32).
114. The dose delivery mechanism (54) of embodiment 113,
wherein the connector (43) is configured to bring the plunger (210) into contact with a bearing (46) located at the piston rod (44) upon the axial movement of the medicament container (48) from the receiving position into the operating position,
wherein, for example, the connector (43) is configured to bring the plunger (210) into contact with the bearing (46) before the medicament container (48) reaches the operating position.
115. A medicament delivery device (10, 300, 500, 700) having a dose delivery mechanism (54, 354, 554, 754) according to one of the preceding embodiments, and a medicament container (48, 348) attached to the dose delivery mechanism (54, 354, 554, 754), the medicament container (48, 348) comprising a plunger (210),
wherein a bearing (46) located at the piston rod (44) is positioned at a predetermined distance with respect to the plunger (210).
116. The medicament delivery device (10, 300, 500, 700) of embodiment 115,
wherein the predetermined distance is zero so that the bearing (46) contacts the plunger (210).
117. The medicament delivery device (10, 300, 500, 700) of embodiment 115,
wherein the predetermined distance is larger than zero.
118. The medicament delivery device (10, 300, 500, 700) of at least embodiments 114and 117,
wherein the predetermined distance is smaller than an axial distance the medicament container (48, 348) is travelling from the receiving position into the operating position.
119. A method for adjusting a position of a piston rod (44) of a dose delivery mechanism (54, 354, 554, 754) for a medicament delivery device (10, 300, 500, 700),
the dose delivery mechanism (54, 354, 554, 754) comprising:
a housing (32, 332, 532);
a piston rod (44); and
an adjusting element (18, 318, 518),
wherein the housing (32, 332, 532) is configured to connect to a medicament container (48, 348) sealed by a plunger (210),
the method comprising:
providing the dose delivery mechanism (54, 354, 554, 754) in a preassembled state;
adjusting an axial position of the piston rod (44) with respect to the housing (32, 332, 532) by rotating the adjusting element (18, 318, 518) in the preassembled state and thereby causing an axial movement of the piston rod (44) with respect to the housing (32, 332, 532); and
transferring the dose delivery mechanism (54, 354, 554, 754) from the preassembled state into an assembled state,
wherein, in the assembled state, the dose delivery mechanism (54, 354, 554, 754) is configured to move the piston rod (44) axially in a proximal direction (1) with respect to the housing (32, 332, 532) during dose delivery such that the piston rod (44) exerts an axial force in the proximal direction (1) on the plunger (210) of the medicament container (48, 348) to expel a medicament from the medicament container (48, 348).
120. The method of embodiment 119,
wherein the dose delivery mechanism (54, 354, 554, 754) is provided in the preassembled state with the medicament container (48, 348) attached.
121. The method of embodiment 120,
wherein the medicament container (48, 348) does not move with respect to the housing (32, 332, 532) upon adjusting the axial position of the piston rod (44).
122. The method of at least one of embodiments 119 to 121,
wherein the axial position of the piston rod (44) is adjusted to place a bearing (46) located at the piston rod (44) in contact with a reference surface.
123. The method of embodiment 122,
wherein the reference surface is provided by a surface of the plunger (210) of the medicament container (48, 348).
124. The method of embodiment 122,
wherein the dose delivery mechanism (54, 354, 554, 754) is provided in the preassembled state without the medicament container (48, 348) attached,
wherein the method further comprises:
placing the dose delivery mechanism (54, 354, 554, 754) in an assembly jig;
wherein the reference surface is provided by a surface of the assembly jig.
125. The method of at least one of embodiments 119 to 124,
wherein the piston rod (44) is axially moved during the adjusting of the axial position until the rotating of the adjusting element (18, 318, 518) requires a predetermined torque.
126. The method of at least one of embodiments 119 to 125,
wherein the method further comprises:
measuring a position of the piston rod (44) with a measurement device for determining an adjusted position of the piston rod (44),
wherein, for example, the measurement device is one of an optical measurement device and a mechanical measurement device, such as an assembly jig having a reference surface.

Furthermore, the present disclosure is directed at the following embodiments:
1. A dose delivery mechanism (54, 354, 554, 754) for a medicament delivery device (10, 300, 500, 700) comprising:
a housing (32, 332, 532);
a piston rod (44); and
an adjusting element (18, 318, 518),
wherein the housing (32, 332, 532) is configured to connect to a medicament container (48, 348) sealed by a plunger (210),
wherein the dose delivery mechanism (54, 354, 554, 754) has a preassembled state and an assembled state,
wherein, in the assembled state, the dose delivery mechanism (54, 354, 554, 754) is configured to move the piston rod (44) axially in a proximal direction (1) with respect to the housing (32, 332, 532) during dose delivery such that the piston rod (44) exerts an axial force in the proximal direction (1) on the plunger (210) of the medicament container (48, 348) to expel a medicament from the medicament container (48, 348), and
wherein, in the preassembled state, the adjusting element (18, 318, 518) is configured to perform a rotation with respect to the housing (32, 332, 532), the rotation causing an axial movement of the piston rod (44) for adjusting an axial position of the piston rod (44) with respect to the housing (32, 332, 532) prior to transfer of the dose delivery mechanism (54, 354, 554, 754) from the preassembled state into the assembled state,
wherein an outer rim (19) of the adjusting element (18, 318, 518) is accessible to an assembler of the device in the preassembled state to effect the rotation of the adjusting element (18, 318, 518) and the axial movement of the piston rod (44),
wherein the adjusting element (18, 318, 518) is configured to perform the rotation at least while being in a preassembled position with respect to the housing (32, 332, 532),
wherein the preassembled position is a most distal position of the adjusting element (18, 318, 518) with respect to the housing (32, 332, 532) in the preassembled state,
wherein the dose delivery mechanism (54, 354, 554, 754) is configured to transfer the rotation of the adjusting element (18, 318, 518) into the axial movement of the piston rod (44) via a single threaded connection (189) in the preassembled state,
wherein the threaded connection (189) comprises a first threaded element (44) that is threadedly engaged with a second threaded element (38, 523).
2. The dose delivery mechanism (354, 554, 754) of embodiment 1,
wherein the dose delivery mechanism (354, 554, 754) comprises a biasing element (250) that biases the adjusting element (318, 518) into the preassembled position with respect to the housing (332, 532) in the preassembled state.
3. The dose delivery mechanism (54, 354, 554, 754) of at least one of the preceding embodiments,
wherein the first and second threaded elements (38, 44, 523) rotate with respect to each other during one of dose setting and dose delivery in the assembled state,
wherein the first and second threaded elements (38, 44, 523) do not rotate with respect to each other during the other one of dose setting and dose delivery in the assembled state.
4. The dose delivery mechanism (554, 754) of embodiment 3,
wherein, during dose delivery in the assembled state, the first threaded element (44) is rotationally stationary with respect to a third element (532) of the dose delivery mechanism (554, 754) and the second threaded element (523) is rotated with respect to the third element (532),
wherein, during adjustment of the piston rod (44) in the preassembled state, the first threaded element (44) is rotated with respect to the third element (532) of the dose delivery mechanism (554, 754) and the second threaded element (523) is rotationally stationary with respect to the third element (532).
5. The dose delivery mechanism (54, 354) of embodiment 3,
wherein, during dose setting in the assembled state, the first threaded element (44) is axially stationary with respect to a third element (32, 332) of the dose delivery mechanism (54, 354) and the second threaded element (38) is axially moved with respect to the third element (32, 332),
wherein, during adjustment of the piston rod (44) in the preassembled state, the first threaded element (44) is axially moved with respect to the third element (32, 332) of the dose delivery mechanism (54, 354) and the second threaded element (38) is axially stationary with respect to the third element (32, 332).
6. The dose delivery mechanism (54, 354) of at least one of the preceding embodiments,
wherein the threaded connection (189) acts between the adjusting element (18, 318) and the piston rod (44).
7. The dose delivery mechanism (54, 354, 554, 754) of at least one of the preceding embodiments,
wherein the dose delivery mechanism (54, 354, 554, 754) comprises a further member (23, 323, 523),
wherein the adjusting element (18, 318, 518) is rotationally decoupled from the further member (23, 323, 523) during adjustment of the piston rod (44) in the preassembled state,
wherein the adjusting element (18, 318, 518) is rotationally coupled to the further member (23, 323, 523) during dose setting in the assembled state,
wherein the adjusting element (18, 318, 518) is rotationally decoupled from the further member (23, 323, 523) during dose delivery in the assembled state.
8. The dose delivery mechanism (54, 354, 554, 754) of embodiment 7,
wherein the dose delivery mechanism (54, 354, 554, 754) comprises a clutch mechanism (107, 507),
wherein the clutch mechanism (107, 507) comprises a first clutch member (22, 520) and a second clutch member (24, 524),
wherein the first clutch member (22, 250) and the second clutch member (24, 524) engage with each other to rotationally couple the adjusting element (18, 318, 518) to the further member (23, 323, 523) in a closed state of the clutch mechanism (107, 507) during dose setting in the assembled state,
wherein the first clutch member (22, 250) and the second clutch member (24, 524) disengage from each other to rotationally decouple the adjusting element (18, 318, 518) from the further member (23, 323, 523) in an opened state of the clutch mechanism (107,507) during dose delivery in the assembled state.
9. The dose delivery mechanism (54, 354) of embodiment 8,
wherein the adjusting element (18) is rotationally decoupled from both the first clutch member (22) and the second clutch member (24) in the preassembled state.
10. The dose delivery mechanism (554, 754) of embodiment 8,
wherein the adjusting element (518) is rotationally fixed with respect to one of the first clutch member (520) and the second clutch member (524) in the preassembled state,
wherein, during adjustment of the position of the piston rod (44) in the preassembled state, the clutch mechanism (107, 507) is in an opened state and the first clutch member (520) and the second clutch member (524) disengage from each other thus allowing a rotation of the adjusting element (18, 318, 518) with respect to the further member (23, 323, 523).
11. The dose delivery mechanism (554) of embodiment 10,
wherein the clutch mechanism (507) comprises a first clutch part and a second clutch part, a first clutch part (508) and a second clutch part (509),
wherein the first clutch part (508) and the second clutch part (509) are engaged with each other in the closed state of the clutch mechanism (507) and disengage from each other in the opened state of the clutch mechanism (507),
wherein the first clutch part (508) is located at a first axial side from the second clutch part (509) in the opened state of the clutch (507) in the preassembled state of the dose delivery mechanism (554),
wherein the first clutch part (508) is located at a second axial side from the second clutch part (509) in the opened state in the assembled state of the dose delivery mechanism (54),
wherein the second axial side is opposite the first axial side.
12. The dose delivery mechanism (54, 354) of at least one of the preceding embodiments,
wherein the adjusting element (18, 318) is rotatable with respect to a counter element (22) in the preassembled state and rotationally fixed, such as irreversibly rotationally fixed, to the counter element (22) in the assembled state.
13. The dose delivery mechanism (54, 354) of embodiment 12,
wherein the counter element (22) is a dose setting element (22) of the dose delivery mechanism (54, 354),
wherein the dose setting element (22) is configured to be gripped by a user of the dose delivery mechanism (54, 354) in the assembled state to set a dose to be delivered.
14. The dose delivery mechanism (54, 354) of at least one of embodiments 12 and 13,
wherein the adjusting element (18, 318) protrudes distally from the counter element (22) in the preassembled state.
15. The dose delivery mechanism (54, 354) of at least one of the preceding embodiments,
wherein the adjusting element (18, 318) is configured to rotate with respect to the piston rod (44) upon the rotation with respect to the housing (32, 332) in the preassembled state.
16. The dose delivery mechanism (554, 754) of at least one of embodiments 1 to 14,
wherein the adjusting element (518) is rotationally fixed with respect to the piston rod (44) in the preassembled state.
17. The dose delivery mechanism (54, 354, 554, 754) of at least one of the preceding embodiments,
wherein the adjusting element (18, 318, 518) is configured to rotate in the assembled state to set a dose of the medicament to be delivered by the dose delivery mechanism (54, 354, 554, 754).
18. The dose delivery mechanism (54, 354, 554, 754) of embodiment 17,
wherein the dose delivery mechanism (54, 354, 554, 754) comprises a dose definition mechanism (115) that defines doses settable by a user in the assembled state,
wherein the dose definition mechanism (115) defines rotational positions of the adjusting element (18, 318, 518) with respect to the housing (32, 332, 532) that correspond to the doses settable by the user,
wherein the dose definition mechanism (115) is not active during the rotation of the adjusting element (18, 318, 518) in the preassembled state.
19. A medicament delivery device (10, 300, 500, 700) having a dose delivery mechanism (54, 354, 554, 754) according to one of the preceding embodiments, and
a medicament container (48, 348) attached to the dose delivery mechanism (54, 354, 554, 754), the medicament container (48, 348) comprising a plunger (210),
wherein a bearing (46) located at the piston rod (44) is positioned at a predetermined distance with respect to the plunger (210).
20. A method for adjusting a position of a piston rod (44) of a dose delivery mechanism (54, 354, 554, 754) for a medicament delivery device (10, 300, 500, 700),
the dose delivery mechanism (54, 354, 554, 754) comprising:
a housing (32, 332, 532);
a piston rod (44); and
an adjusting element (18, 318, 518),
wherein the housing (32, 332, 532) is configured to connect to a medicament container (48, 348) sealed by a plunger (210),
the method comprising:
providing the dose delivery mechanism (54, 354, 554, 754) in a preassembled state,
wherein an outer rim (19) of the adjusting element (18, 318, 518) is accessible to an assembler of the device in the preassembled state to effect rotation of the adjusting element (18, 318, 518) and axial movement of the piston rod (44);
adjusting, in the preassembled state, an axial position of the piston rod (44) with respect to the housing (32, 332, 532) by rotating the adjusting element (18, 318, 518) in the preassembled state and thereby causing an axial movement of the piston rod (44) with respect to the housing (32, 332, 532),
wherein the rotation of the adjusting element (18, 318, 518) is transferred into the axial movement of the piston rod (44) via a single threaded connection (189) of the dose delivery mechanism (54, 354, 554); and
transferring the dose delivery mechanism (54, 354, 554, 754) from the preassembled state into an assembled state,
wherein, in the assembled state, the dose delivery mechanism (54, 354, 554, 754) is configured to move the piston rod (44) axially in a proximal direction (1) with respect to the housing (32, 332, 532) during dose delivery such that the piston rod (44) exerts an axial force in the proximal direction (1) on the plunger (210) of the medicament container (48, 348) to expel a medicament from the medicament container (48, 348).

1. A dose delivery mechanism for a medicament delivery device comprising:
a housing;
a piston rod; and
an adjusting element,
the housing configured to connect to a medicament container sealed by a plunger,
the dose delivery mechanism is in a preassembled state, and
in the preassembled state, the adjusting element is configured to adjust an axial position of the piston rod with respect to the housing upon a rotation of the adjusting element with respect to the housing,
the dose delivery mechanism is configured to transfer from the preassembled state into an assembled state,
in the assembled state, the dose delivery mechanism is configured to move the piston rod axially in a proximal direction with respect to the housing during dose delivery such that the piston rod exerts an axial force in the proximal direction on the plunger of the medicament container to expel a medicament from the medicament container,
the adjusting element comprising a radially outermost surface is configured to be gripped by a user to set a dose to be delivered in the assembled state, and
the adjusting element completely comprising the radially outermost surface.

2. The dose delivery mechanism of claim 1,
wherein the adjusting element is configured to perform the rotation in the preassembled state after having been displaced from a rest position into an adjusting position that deviates from the rest position, and
the adjusting element is configured to be pushed in a proximal direction from the rest position into the adjusting position.

3. The dose delivery mechanism of claim 1,
wherein the dose delivery mechanism is configured to transfer the rotation of the adjusting element into axial movement of the piston rod via a single threaded connection in the preassembled state,
the single threaded connection comprises a first threaded element that is threadedly engaged with a second threaded element, and
a pitch of the single threaded connection defines a ratio of an axial distance to a circumferential distance and the piston rod travels the axial distance with respect to the housing upon rotation of the adjusting element by the circumferential distance.

4. The dose delivery mechanism of claim 1,
wherein an outer rim of the adjusting element is not covered by the housing of the dose delivery mechanism, in a radial direction perpendicular to a longitudinal axis of the dose delivery mechanism.

5. The dose delivery mechanism of claim 1,
wherein, in the preassembled state, the adjusting element is configured to perform the rotation with respect to the housing, and
the rotation of the adjusting element causes an axial movement of the piston rod to adjust the axial position of the piston rod with respect to the housing prior to transfer of the dose delivery mechanism from the preassembled state into the assembled state.

6. The dose delivery mechanism of claim 1,
wherein the dose delivery mechanism is configured to be assembled from the same components in the preassembled state and in the assembled state.

7. The dose delivery mechanism of claim 1,
wherein the transfer of the dose delivery mechanism from the preassembled state into the assembled state does not comprise an addition or removal of a component of the dose delivery mechanism.

8. The dose delivery mechanism of claim 1,
wherein all remaining components of the dose delivery mechanism except the adjusting element, the piston rod and components axially fixed to the piston rod are axially positioned in a zero dose position with respect to the housing in the preassembled state, and
the remaining components are configured to take up the zero dose position prior to setting a dose with the dose delivery mechanism in the assembled state.

9. The dose delivery mechanism of claim 1,
wherein the adjusting element is configured to be transferred from a preassembled position into an assembled position with respect to the housing when transferring the dose delivery mechanism from the preassembled state into the assembled state, and
the transfer of the adjusting element from the preassembled position into the assembled position causes a transfer of the dose delivery mechanism from the preassembled state into the assembled state.

10. The dose delivery mechanism of claim 1,
wherein the adjusting element is in a first axial position with respect to a retaining member of the dose delivery mechanism in the preassembled state, and
the adjusting element is configured to move axially from the first axial position into a second axial position with respect to the retaining member upon transfer of the dose delivery mechanism from the preassembled state into the assembled state.

11. The dose delivery mechanism of claim 10,
wherein the retaining member is formed by a coupling element configured to rotationally fix the adjusting element to the piston rod.

12. The dose delivery mechanism of claim 10,
wherein the adjusting element is rotationally fixed with respect to the retaining member in the preassembled state.

13. The dose delivery mechanism of claim 10,
wherein the retaining member is configured to be moved axially together with the adjusting element into an adjusting position with respect to the housing, and
the adjusting element is configured to perform the rotation with respect to the housing in the adjusting position to cause axial movement of the piston rod.

14. The dose delivery mechanism of at least claim 13,
wherein the axial movement of the retaining member opens a clutch mechanism to rotationally decouple a further member from the adjusting element during the rotation with respect to the housing in the adjusting position,
the further member is configured to threadedly couple to the piston rod or be axially fixed with respect to the housing, and
the further member is formed by a dosing member of the dose delivery mechanism.

15. The dose delivery mechanism of claim 13,
wherein the adjusting element is configured to be moved axially into a proximal direction when being in the adjusting position, and
further proximal movement of the adjusting element from an adjusting position transfers the adjusting element from the first axial position into the second axial position with respect to the retaining member and transfers the dose delivery mechanism from the preassembled state into the assembled state.

16. The dose delivery mechanism of claim 10,
wherein the adjusting element is axially coupled to the retaining member via a latching mechanism.

17. The dose delivery mechanism of claim 16,
wherein the latching mechanism is configured to transfer axial movement of the adjusting element into axial movement of the retaining member during movement of the adjusting element.

18. The dose delivery mechanism of claim 16,
wherein the latching mechanism is configured to allow proximal movement of the adjusting element with respect to the retaining member in an adjusting position to transfer the dose delivery mechanism from the preassembled state into the assembled state.

19. The dose delivery mechanism of claim 1,
further comprising a clutch mechanism having a first clutch member and a second clutch member,
the clutch mechanism connecting the adjusting element to a further member,
the clutch mechanism is opened in the preassembled state during the rotation of the adjusting element thus allowing rotation of the adjusting element with respect to the further member, and
the clutch mechanism is opened in the assembled state during dose delivery thus allowing the rotation of the adjusting element with respect to the further member.

20. The dose delivery mechanism of claim 19,
wherein the clutch mechanism is closed in the assembled state during dose setting.

21. The dose delivery mechanism of claim 19,
wherein the clutch mechanism is biased into a closed configuration in the preassembled state, and
the adjusting element is configured to be axially moved with respect to the housing in the preassembled state to open the clutch mechanism.

22. The dose delivery mechanism of claim 1,
further comprising a dose definition mechanism defining doses settable by a user in the assembled state, and
the dose definition mechanism defines rotational positions of the adjusting element with respect to the housing that correspond to doses settable by the user.

23. The dose delivery mechanism of at least claim 22,
wherein the dose definition mechanism is actuated during the rotation of the adjusting element in the preassembled state.

24. The dose delivery mechanism of claim 1,
wherein a distal end surface of the dose delivery mechanism forms part of the adjusting element or is rigidly connected with the adjusting element.

25. The dose delivery mechanism of claim 1,
wherein the adjusting element is located at a distal end of the dose delivery mechanism.

26. The dose delivery mechanism of claim 1,
wherein the adjusting element forms at least part of a knob that is positioned at a distal end of the dose delivery mechanism.

27. The dose delivery mechanism of claim 1,
wherein the adjusting element protrudes in a distal direction from the housing in the preassembled state, and
the adjusting element protrudes in a distal direction from remaining members of the dose delivery mechanism in the preassembled state.

28. A medicament delivery device, comprising:
the dose delivery mechanism according to claim 1; and
a medicament container attached to the dose delivery mechanism, the medicament container comprising a plunger, and
a bearing located at the piston rod is positioned at a predetermined distance with respect to the plunger, and
the predetermined distance is larger than zero.

29. The medicament delivery device of claim 28,
wherein the medicament container comprises a medicament chamber that is sealed by the plunger, and
the medicament chamber comprises a medicament and air.

30. A method for adjusting a position of a piston rod of a dose delivery mechanism according to claim 1,
the method comprising:
providing the dose delivery mechanism in the preassembled state;
adjusting the axial position of the piston rod with respect to the housing by rotating the adjusting element in the preassembled state and thereby causing an axial movement of the piston rod with respect to the housing; and
transferring the dose delivery mechanism from the preassembled state into the assembled state.

31. The method of claim 30,
further comprising
moving the adjusting element from a rest position with respect to the housing into an adjusting position,
the rotating the adjusting element is performed with the adjusting element being in the adjusting position.

32. The method of claim 30,
wherein the adjusting element is moved axially in a proximal direction from the preassembled position into the adjusting position.

33. The method of claim 30,
wherein the dose delivery mechanism comprises a stop acting between the adjusting element and the housing, and
the stop exerts a resistance against the moving of the adjusting element when the adjusting element reaches the adjusting position.

34. The method of at least claim 33, further comprising:
overcoming the resistance exerted by the stop to further move the adjusting element to transfer the dose delivery mechanism from the preassembled state into the assembled state.

35. The method of claim 31,
wherein the adjusting element is biased into the preassembled position by a biasing force,
the method further comprises: comprising
holding the adjusting element in the adjusting position against the biasing force during the rotating of the adjusting element.

36. The method of claim 30,
wherein the dose delivery mechanism comprises a clutch mechanism,
clutch mechanism is closed when the adjusting element is in the preassembled state, and
the moving of the adjusting element from the preassembled position into the adjusting position opens the clutch mechanism.

37. The method of claim 1,
wherein the dose delivery mechanism includes the adjusting element in a first axial position with respect to a retaining member of the dose delivery mechanism, and
the moving of the adjusting element from the preassembled position into the adjusting position causes the retaining member to axially move together with the adjusting element-.

38. The method of claim 30,
wherein the dose delivery mechanism is provided in the preassembled state with all components of the dose delivery mechanism connected to each other.

39. The method of claim 30,
wherein the dose delivery mechanism comprises a dose definition mechanism defining doses settable by a user in an assembled state of the dose delivery mechanism, and
the dose definition mechanism defines rotational positions of the adjusting element with respect to the housing that correspond to the doses settable by the user,
the method further comprises: comprising
actuating the dose definition mechanism during the rotating of the adjusting element in the preassembled state, to generate a feedback signal,

40. The method of claim 30,
wherein the dose delivery mechanism is provided in the preassembled state with the medicament container attached.

41. The method of at least claim 40,
wherein the axial position of the piston rod is adjusted to place a bearing located at the piston rod in contact with a surface of the plunger of the medicament container.

42. The method of at claim 40,
wherein the axial position of the piston rod is adjusted to move the plunger within the medicament container to expel air from the medicament container.

43. The method of claim 30,
wherein the piston rod is axially moved during the adjusting of the axial position until the rotating of the adjusting element requires a predetermined torque.

44. The method of claim 30, further comprising:
measuring a torque required for the rotating of the adjusting element.

45. The method of claim 30, further comprising
transferring the adjusting element from a preassembled position into an assembled position.

46. The method of at least claim 45, further comprising
blocking the adjusting element in the assembled position from returning into the preassembled position with respect to the housing.

47. The method of claim 30, further comprising
transferring the adjusting element from a first axial position with respect to a retaining member of the dose delivery mechanism into a second axial position with respect to the retaining member to transfer the dose delivery mechanism from the preassembled state into the assembled state.

48. A method for assembling the dose delivery mechanism according to claim 1,
the method comprising:
connecting the adjusting element to a retaining member of the dose delivery mechanism so that the adjusting element is in a first axial position with respect to the retaining member,
the adjusting element configured to move axially from the first axial position into a second axial position with respect to the retaining member upon transfer of the dose delivery mechanism from the preassembled state into the assembled state; and
providing the dose delivery mechanism in the preassembled state and with the adjusting element in the first axial position with respect to the retaining member so that the position of the piston rod is adjustable by rotating the adjusting element.

49. The method of claim 48,
further comprising
keeping the adjusting element in the first axial position until the axial position of the piston rod has been adjusted by rotating the adjusting element.

50. The method of claim 48,
wherein the connecting the adjusting element to the retaining member includes connecting the adjusting element to remaining components of the dose delivery mechanism.

51. The method of claim 48, further comprising
positioning the adjusting element in a preassembled position with respect to the housing.

52. The method of at least claim 51, further comprising
biasing the adjusting element into the preassembled position with respect to the housing by a biasing element.

53. The method of claim 48,
further comprising
positioning remaining components of the dose delivery mechanism except for the adjusting element, the piston rod and components axially fixed to the piston rod axially in a respective zero dose position with respect to the housing, and
the remaining components are configured to be in a respective zero dose position prior to setting a dose with the dose delivery mechanism in the assembled state.

54. The method of claim 48, further comprising
providing a subassembly comprising a coupling member and a coupling element,
movement of the coupling member is restrained in the axial direction with respect to the coupling element; and
attaching the subassembly to the housing.

55. The method of at least claim 54,
wherein the subassembly further comprises an insert,
the insert is axially fixed to the coupling member and rotationally movable with respect to the insert.

56. The method of at claim 54, wherein the coupling element forms the retaining member.
